SEQUENCE LISTING (29 sequences)

SEQ ID NO: 1 - DNA, 41 bp, synthetic construct
  source 1..41: mol_type = other DNA
acaaaggcagccacgcactccttccctgaaggccggaatct

SEQ ID NO: 2 - DNA, 41 bp, synthetic construct
  source 1..41: mol_type = other DNA
gctgccgccactagccatcttactgctgaggactcttcgct

SEQ ID NO: 3 - DNA, 28 bp, synthetic construct
  source 1..28: mol_type = other DNA
acaaaggcagccacgcactccttccctg

SEQ ID NO: 4 - DNA, 29 bp, synthetic construct
  source 1..29: mol_type = other DNA
acaaaggcagccacgcactccttccctgt

SEQ ID NO: 5 - DNA, 40 bp, synthetic construct
  source 1..40: mol_type = other DNA
gattccggccttgtggttggtgagggtcatctcgctggag

SEQ ID NO: 6 - DNA, 40 bp, synthetic construct
  source 1..40: mol_type = other DNA
gcgaagagtcctggagtgccgccaatgtatgcgagggtga

SEQ ID NO: 7 - DNA, 28 bp, synthetic construct
  source 1..28: mol_type = other DNA
gtggttggtgagggtcatctcgctggag

SEQ ID NO: 8 - DNA, 29 bp, synthetic construct
  source 1..29: mol_type = other DNA
agtggttggtgagggtcatctcgctggag

SEQ ID NO: 9 - DNA, 71 bp, synthetic construct
  source 1..71: mol_type = other DNA
gcgcgcgttttttttgcttgcgtctcctgccagccatatccggtctacgtgatccttttttttcgcgcgct

SEQ ID NO: 10 - DNA, 83 bp, synthetic construct
  source 1..83: mol_type = other DNA
gcgcgcgttttttttttttttgcttgcgtctcctgccagccatatccggtctacgtgatccttttttttttttttcgcgcgct

SEQ ID NO: 11 - DNA, 62 bp, synthetic construct
  source 1..62: mol_type = other DNA
ggatcacgtagattttgcttgcgtctcctgccagccatatccggtttttctacgtgattcct

SEQ ID NO: 12 - DNA, 81 bp, synthetic construct
  source 1..81: mol_type = other DNA
gcgaagagtcctggagtgccgccaatgtatgcgagggtgagctgccgccactagccatcttactgctgaggactcttcgct

SEQ ID NO: 13 - DNA, 93 bp, synthetic construct
  source 1..93: mol_type = other DNA
gcgaagagtcctttttttggagtgccgccaatgtatgcgagggtgagctgccgccactagccatcttactgctgttttttaggactcttcgct

SEQ ID NO: 14 - DNA, 100 bp, synthetic construct
  source 1..100: mol_type = other DNA
gcgaagagtcctttttttggagtgccgccaatgtatgcgagggtgatttttttgctgccgccactagccatcttactgctgttttttaggactcttcgct

SEQ ID NO: 15 - DNA, 81 bp, synthetic construct
  source 1..81: mol_type = other DNA
gattccggccttgtggttggtgagggtcatctcgctggagacaaaggcagccacgcactccttccctgaaggccggaatct

SEQ ID NO: 16 - DNA, 93 bp, synthetic construct
  source 1..93: mol_type = other DNA
gattccggccttttttttgtggttggtgagggtcatctcgctggagacaaaggcagccacgcactccttccctgttttttaaggccggaatct

SEQ ID NO: 17 - DNA, 100 bp, synthetic construct
  source 1..100: mol_type = other DNA
gattccggccttttttttgtggttggtgagggtcatctcgctggagtttttttacaaaggcagccacgcactccttccctgttttttaaggccggaatct

SEQ ID NO: 18 - DNA, 61 bp, synthetic construct
  source 1..61: mol_type = other DNA
ggatcacgtagattttgcttgcgtctcctgccagccatatccggtttttctacgtgatcct

SEQ ID NO: 19 - DNA, 77 bp, synthetic construct
  source 1..77: mol_type = other DNA
ggatcacgtagattttttttttttgcttgcgtctcctgccagccatatccggtttttttttttttctacgtgatcct

SEQ ID NO: 20 - DNA, 101 bp, synthetic construct
  source 1..101: mol_type = other DNA
ggatcacgtagattttttttttttttttttttttttgcttgcgtctcctgccagccatatccggtttttttttttttttttttttttttctacgtgatcct

SEQ ID NO: 21 - DNA, 103 bp, synthetic construct
  source 1..103: mol_type = other DNA
ggatcacgtagattttttttttttttttttttttttttttttttttttttttgcttgcgtctcctgccagccatatccggtttttttttttctacgtgatcct

SEQ ID NO: 22 - DNA, 67 bp, synthetic construct
  source 1..67: mol_type = other DNA
ggatcacgtagattttagatctgcttgcgtctcctgccagccatatccggtttttctacgtgatcct

SEQ ID NO: 23 - DNA, 82 bp, synthetic construct
  source 1..82: mol_type = other DNA
ggatcacgtagattttttttttttagatctgcttgcgtctcctgccagccatatccggttttttttttttctacgtgatcct

SEQ ID NO: 24 - DNA, 56 bp, synthetic construct
  source 1..56: mol_type = other DNA
gtggttggtgagggtcatctcgctggagacaaaggcagccacgcactccttccctg

SEQ ID NO: 25 - DNA, 68 bp, synthetic construct
  source 1..68: mol_type = other DNA
gtggttggtgagttttttggtcatctcgctggagacaaaggcagccacgttttttcactccttccctg

SEQ ID NO: 26 - DNA, 75 bp, synthetic construct
  source 1..75: mol_type = other DNA
gtggttggtgagttttttggtcatctcgctggagtttttttacaaaggcagccacgttttttcactccttccctg

SEQ ID NO: 27 - DNA, 58 bp, synthetic construct
  source 1..58: mol_type = other DNA
agtggttggtgagggtcatctcgctggagacaaaggcagccacgcactccttccctgt

SEQ ID NO: 28 - DNA, 70 bp, synthetic construct
  source 1..70: mol_type = other DNA
agtggttggtgattttttgggtcatctcgctggagacaaaggcagccacgcttttttactccttccctgt

SEQ ID NO: 29 - DNA, 76 bp, synthetic construct
  source 1..76: mol_type = other DNA
agtggttggtgattttttggtcatctcgctggagtttttttacaaaggcagccacgcttttttactccttccctgt

(19) United States
(12) Patent Application Publication
(10) Pub. No.: US 2025/0154555 A1
(43) Pub. Date: May 15, 2025

(54) NUCLEIC ACID AMPLIFICATION AND METHYLATION PATTERN RETENTION

(71) Applicant: Singular Genomics Systems, Inc., San Diego, CA (US)

(72) Inventors: Daan Witters, San Diego, CA (US); Eli N. Glezer, Del Mar, CA (US)

(21) Appl. No.: 18/836,312

(22) PCT Filed: Feb. 10, 2023
(86) PCT No.: PCT/US2023/062437

§ 371 Date: Aug. 6, 2024

Related U.S. Application Data
(60) Provisional application No. 63/309,373, filed on Feb. 11, 2022.

(51) Int. Cl. C12Q 1/6806; C12Q 1/48; C12Q 1/6844

(52) U.S. Cl. CPC C12Q 1/6806; C12Q 1/485; C12Q 1/6844

(57) ABSTRACT

Disclosed herein, inter alia, are compositions, methods, and kits useful for detecting nucleobase modifications on one or both strands of a double-stranded nucleic acid fragment.

### CROSS-REFERENCES TO RELATED APPLICATIONS

This application claims the benefit of U.S. Provisional Application No. 63/309,373, filed Feb. 11, 2022, which is incorporated herein by reference in its entirety and for all purposes.

### REFERENCE TO SEQUENCE LISTING SUBMITTED ELECTRONICALLY

The Sequence Listing titled 051385-566001WO_ST26.XML, was created on Jan. 17, 2023 in machine format IBM-PC, MS-Windows operating system, is 27,956 bytes in size, and is hereby incorporated by reference in its entirety for all purposes.

### BACKGROUND

Liquid biopsies show promise for early detection and therapeutic interventions for metastatic cancers. Repeated tissue biopsies of metastatic lesions are invasive and, depending on the location of the cancerous tissue, difficult to access. In contrast, monitoring of biological samples (e.g., blood, plasma, or other bodily fluids) is minimally invasive and can identify tumor evolution and tumor subtype switches, which may then lead to the selection of appropriate therapies. For example, liquid biopsies analyzing the circulating tumor DNA (ctDNA) found in blood, pancreatic cysts, Pap smears, urine, stool, and saliva have been performed. Epigenetic information, such as biomolecule methylation, and/or -additional protein biomarkers combined with cfDNA and ctDNA analyses, is useful in determining the origin of cancer at an early stage. Biomolecule methylation, such as DNA methylation, is widespread and plays a critical role in the regulation of gene expression in development, differentiation, and disease. The sequence context of methylation patterns is critical on the mutational pattern seen in cancer, but existing sequencing technologies fail to retain the complexity of methylation patterns. Disclosed herein, inter alia, are solutions to these and other problems in the art.

### BRIEF SUMMARY

In an aspect is provided compositions, kits, and methods for differentiating modifications to nucleobases (e.g., discerning chemical modifications to cytosine nucleobases) within double-stranded nucleic acids.

In an aspect is provided a method of generating an immobilized methylated complement template polynucleotide, the method including: i) hybridizing a methylated template polynucleotide to a first immobilized primer at a first temperature, wherein the first immobilized primer is attached to a solid support; ii) extending the first immobilized primer with a polymerase to generate an immobilized non-methylated complement template polynucleotide hybridized to the methylated template polynucleotide; and iii) contacting the immobilized non-methylated complement template polynucleotide with a DNA methyltransferase reagent to generate an immobilized methylated complement template polynucleotide, wherein the methylated complement template polynucleotide includes one or more methylated cytosine nucleobases and one or more non-methylated cytosine nucleobases.

In an aspect is provided an immobilized polynucleotide including a first nucleic acid sequence including one or more methylated cytosine nucleobases hybridized to a second nucleic acid sequence including one or more uracil nucleobases, wherein the polynucleotide includes one or more cytosine mismatches, and wherein the immobilized polynucleotide is attached to a solid support.

### BRIEF DESCRIPTION OF THE DRAWINGS

FIGS. 1A-1B. FIG. 1A shows an embodiment of an adapter-target-adapter template including a double stranded nucleic acid of interest annealed to a Y-adapter and a hairpin adapter. FIG. 1B shows an embodiment of an adapter-target-adapter template where a double stranded nucleic acid of interest is annealed to a first hairpin adapter (hairpin adapter 1) and a second, non-identical, hairpin adapter (hairpin adapter 2). Primer binding sites (i.e., sequences having complementarity to a specific primer) are identified as P1, P2′, and P3 indicating unique primer binding sites, or complements thereof.

FIGS. 2A-2D show embodiments of adapters. FIG. 2A shows an embodiment of a Y adapter including (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand. In this embodiment, the complementary portions (i.e., duplex regions) of the Y adapter include a melting temperature (Tm) of about 40-45° C. and a length of about 10 to 15 nucleotides. In embodiments, the complementary portions (i.e., duplex regions) of the Y adapter include a Tm (melting temperature) of about 35-45° C. or 30-45° C. and a length of about 12 bases. In embodiments, the single-stranded portions (i.e., 3′-arm and 5′-arm regions) of the Y adapter include a Tm (melting temperature) of about 50-75° C. FIG. 2B shows an embodiment of a hairpin adapter including a 5′-end, a 5′ portion, a loop, a 3′ portion and a 3′-end. In this embodiment, a duplex region of the hairpin adapter includes a Tm (melting temperature) of about 40-45° C. and a length of about 10-16 bases. In embodiments, the duplex region of the adapter includes a Tm (melting temperature) of about 35-45° C. or 30-45° C. and a length of about 12 bases. FIG. 2C illustrates an embodiment of a hairpin adapter, which includes a double stranded (stem) region and a loop region. Within the loop region is a priming site (P3) and optionally a unique molecular identifier (UMI). FIG. 2D illustrates the adapters may include different duplex ends. For example, the double-stranded region of a Y adapter (alternatively referred to as a forked adapter) may be blunt-ended (top), have a 3′ overhang (middle), or a 5′ overhang (bottom). On the right are embodiments of hairpin adapters, each including a 5′-end and a 3′-end. In some embodiments, a hairpin adapter includes a double stranded portion (a double-stranded “stem” region) and a loop, where 5′P refers to a phosphorylated 5′ end. A double-stranded stem region of a hairpin adapter may be blunt-ended (top), it may have a 5′ overhang (middle), or a 3′ overhang (bottom). An overhang may include a single nucleotide or more than one nucleotide.

FIGS. 3A-3B illustrate embodiments for conversion of nucleobases. FIG. 3A illustrates bisulfite conversion, which converts a cytosine nucleobase to a uracil nucleobase (top), however modified cytosine nucleobases (e.g., 5-methylcytosine (5mC) or 5-hydroxymethyl cytosine (5hmC)) are not susceptible to bisulfite conversion methods (bottom). FIG. 3B illustrates an alternate conversion approach, which combines a first enzymatic conversion of a modified cytosine nucleobase (e.g., 5-methylcytosine (5mC) or 5-hydroxymethyl cytosine (5hmC)) to an intermediate nucleobase, 5-carboxylcytosine (5caC), followed by a subsequent conversion to a uracil analog nucleobase, dihydrouridine (DHU).

FIGS. 4A-4C provides an overview of different single-stranded conversion approaches. FIG. 4A illustrates a portion of a single-stranded polynucleotide sequence that contains modified cytosines and non-modified cytosines, e.g., 5′-[A][T][^{5hm}C][A][C][^{5mC}]-3′-, where [^{5hm}C] refers to 5-hydroxymethyl cytosine and [^{5m}C] refers to 5-methylcytosine. Following a first chemical conversion step (e.g., bisulfite conversion) the non-modified cytosine nucleobases are converted to uracil nucleobases. When this single-stranded polynucleotide is subjected to standard amplification methods (e.g., PCR), the resulting polynucleotide includes thymidine nucleobases in the positions where the non-modified cytosine nucleobases were in the original polynucleotide sequence. FIG. 4B depicts a combination of enzymatic and chemical conversion protocols (e.g., TAPS (TET-assisted pyridine borane sequencing)) that convert the modified cytosine nucleobases. Briefly, a portion of a single-stranded polynucleotide sequence that contains modified cytosines and non-modified cytosines, e.g., 5′-[A][T][^{5hm}C][A][C][^{5m}C]-3′-, is subjected to a first enzymatic conversion (e.g., a TET (ten-eleven translocation methylcytosine dioxygenase) enzyme conversion), which converts the modified cytosine nucleobases to an intermediate nucleobase, 5-carboxylcytosine (5caC). A chemical conversion (e.g., contacting the polynucleotide with borane derivatives (e.g., pyridine borane and 2-picoline borane), which converts the 5caC nucleobases to a uracil analog nucleobase, dihydrouridine (DHU). Following standard amplification protocols (e.g., PCR), the DHU nucleobases are amplified as thymidine nucleobases and the non-modified cytosine nucleobases are amplified as cytosines. FIG. 4C depicts an alternate enzymatic approach. Similar to the approach described in the beginning of FIG. 4B, a portion of a single-stranded polynucleotide sequence that contains modified cytosines and non-modified cytosines, e.g., 5′-[A][T][^{5hm}C][A][C][^{5m}C]-3′-, is subjected to a first enzymatic conversion (e.g., a TET enzyme conversion), which converts the modified cytosine nucleobases to an intermediate nucleobase, 5-carboxylcytosine (5caC). A second enzymatic conversion (e.g., APOBEC enzyme conversion) converts the non-modified cytosine nucleobases to uracil nucleobases. When this single-stranded polynucleotide is subjected to standard amplification methods (e.g., PCR), the resulting polynucleotide includes thymidine nucleobases in the positions where the non-modified cytosine nucleobases were in the original polynucleotide sequence and the modified cytosine nucleobases are amplified as non-modified cytosines.

FIGS. 5A-5C presents an embodiment of an amplification method for methylome analysis. FIG. 5A shows a Y-template-hairpin construct containing modified cytosine nucleobases (depicted as triangles) hybridizing to an immobilized P2 primer. In the presence of a polymerase, a first extension is performed, immobilizing a copy of the original template, wherein the copy has guanine nucleobases paired with the modified cytosine nucleobases (i.e., hemi-methylated double-stranded DNA). A methyltransferase is then introduced (e.g., a DNMT1 methyltransferase), along with a methyl donor compound (e.g., S-adenosyl-L-methionine) to generate methyl groups on the extended strand that match the methylation pattern of the original template strand. FIG. 5B shows subsequent denaturation and washing away of the original template strand, allowing rehybridization of the immobilized, methyltransferase-treated strand into a Y-template-hairpin construct A conversion technique may be applied as known in the art and described herein. For example, the enzymatic and chemical conversion method depicted in FIG. 4A may be applied, which converts the unmodified cytosine nucleobases to uracil analogs (depicted as squares). FIG. 5C shows an immobilized P1 primer annealed to the immobilized template polynucleotide. In the presence of a polymerase, an extension is performed. As in FIG. 5A, a methyltransferase is then introduced to generate methyl groups on the extended strand that match the methylation pattern of the original template strand. The process is then repeated to continue amplification of the template polynucleotide while retaining the original methylation information in the amplicons.

FIGS. 6A-6D presents an embodiment of an amplification method for methylome analysis. FIG. 6A shows a Y-template-Y construct containing modified cytosine nucleobases (depicted as triangles) hybridizing to an immobilized P2 primer. In the presence of a polymerase, a first extension is performed, immobilizing a copy of the original template, wherein the copy has guanine nucleobases paired with the modified cytosine nucleobases (i.e., hemi-methylated double-stranded DNA). A methyltransferase is then introduced (e.g., a DNMT1 methyltransferase), along with a methyl donor compound (e.g., S-adenosyl-L-methionine) to generate methyl groups on the extended strand that match the methylation pattern of the original template strand. A conversion technique may be applied as known in the art and described herein. For example, the enzymatic and chemical conversion method depicted in FIG. 4A may be applied, which converts the unmodified cytosine nucleobases to uracil analogs (depicted as squares). FIG. 6B shows a single-stranded DNA template containing modified cytosine nucleobases (depicted as triangles) hybridizing to an immobilized P2 primer and being processed as in FIG. 6A. The converted templates of FIGS. 6A and 6B may then be taken through an amplification process including a methyltransferase step as in FIG. 5C using immobilized P1 and P2 primers, thereby retaining the original methylation pattern in the template strands. Alternative, FIG. 6C shows an immobilized P1 primer annealed to the immobilized template polynucleotide. In the presence of a polymerase, an extension is performed. No methyltransferase step is performed following extension. The strands are then denatured and re-annealed to immobilized P1 and P2 primers, as shown in FIG. 6D. The process is then repeated to continue amplification of the template polynucleotide.

FIGS. 7A-7B illustrate an alternate embodiment of an amplification method for methylome analysis. FIG. 7A illustrates a template nucleic acid containing a first Y adapter, a double-stranded nucleic acid, and a hairpin adapter. The template nucleic acid has been immobilized and methylated as described in FIG. 5A. The double-stranded nucleic acid includes modified cytosine nucleobases, illustrated as triangles on both strands of the nucleic acid. In this embodiment, a primer anneals to the loop region of the hairpin and is extended by a strand-displacing polymerase (depicted as the grey ellipse) to generate a blocking strand. The blocking strand is hybridized to one of the two strands of the double-stranded nucleic acid, whereas the other strand is rendered single-stranded (FIG. 7B). Now that one of the two strands from the original dsDNA template is liberated, a conversion technique may be applied as known in the art and described herein. For example, the enzymatic and chemical conversion method depicted in FIG. 4A may be applied, which converts the modified cytosine nucleobases (depicted as triangles) to uracil analogs (depicted as squares) as shown in FIG. 7B. Once the blocking strand is removed, the template nucleic acid may reanneal, providing a template nucleic acid with asymmetric modifications (i.e., one strand contains modified cytosine nucleobases and the other strand contains converted cytosines (e.g., uracil analogs)). Solid phase DNA amplification may then be performed, for example, as depicted in FIG. 5C.

FIGS. 8A-8B presents an embodiment of a linked duplex sequencing process. FIG. 8A illustrates a nucleic acid template containing a first Y adapter, a double-stranded nucleic acid, and a hairpin adapter, wherein the hairpin adapter contains a cleavable site (i.e., the cleavable site is indicated as ‘X’). The template nucleic acid has been immobilized and methylated as described in FIG. 5A. The double-stranded nucleic acid includes modified cytosine nucleobases, illustrated as triangles on both strands of the nucleic acid. In this embodiment, a runoff primer anneals to the loop region of the hairpin and is extended by a strand-displacing polymerase (depicted as the grey ellipse) to generate an invasion strand. The invasion strand is hybridized to one of the two strands of the double-stranded nucleic acid, whereas the other strand is rendered single-stranded. A sequencing primer S1 is then hybridized to the single-stranded end. FIG. 8B shows sequencing with detectable nucleotides (indicated by the star) from the S1 primer, followed by cleavage at the cleavable site, denaturation, and washing to leave behind the immobilized single-stranded strand. A conversion technique may be applied as known in the art and described herein. For example, the enzymatic and chemical conversion method depicted in FIG. 4A may be applied, which converts the modified cytosine nucleobases (depicted as triangles) to uracil analogs (depicted as squares). A sequencing primer S2 is then hybridized and a second read sequenced (indicated by the star), wherein the second read includes sequencing the converted nucleobases.

### DETAILED DESCRIPTION

The compositions and methods described herein provide solid-phase amplification techniques that maintain the methylation status and patterns of the template nucleic acid within the amplification products. The compositions and methods described herein improve sequencing accuracy by ensuring that the methylated nucleobases are retained in the amplification products, thereby maintaining the true methylation status of the starting nucleic acid material.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. The term “immobilized”, as used herein, refers to the association, attachment, or binding between a molecule (e.g. linker, adapter, oligonucleotide) and a solid support in a manner that provides a stable association under the conditions of elongation, amplification, ligation, and other processes as described herein. Such binding can be covalent or non-covalent. Non-covalent binding includes electrostatic, hydrophilic and hydrophobic interactions. Covalent binding is the formation of covalent bonds that are characterized by sharing of pairs of electrons between atoms. Such covalent binding can be directly between the molecule and the solid support or can be formed by a cross linker or by inclusion of a specific reactive group on either the solid support or the molecule or both. Covalent attachment of a molecule can be achieved using a binding partner, such as avidin or streptavidin, immobilized to the solid support and the non-covalent binding of the biotinylated molecule to the avidin or streptavidin. Immobilization may also involve a combination of covalent and non-covalent interactions.

As used herein, the term “3′ end” designates the end of a nucleotide strand that has the hydroxyl group of the third carbon in the sugar-ring of the deoxyribose at its terminus.

As used herein, the term “5′ end” designates the end of a nucleotide strand that has the fifth carbon in the sugar-ring of the deoxyribose at its terminus.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a particle described herein to interact with an array.

As may be used herein, the terms “nucleic acid,” “nucleic acid molecule,” “nucleic acid sequence,” “strand”, “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analog. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the intemucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analog, via an 8-modified purine, via an N-6 modified adenosine or an N-2 modified guanine. For pyrimidines, attachment is preferably via the 5-position on cytidine, thymidine or uracil and the N4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. Cleavage agents used in methods described herein may be selected from nicking endonucleases, DNA glycosylases, or any single-stranded cleavage agents described in further detail elsewhere herein. Enzymes for cleavage of single-stranded DNA may be used for cleaving heteroduplexes in the vicinity of mismatched bases, D-loops, heteroduplexes formed between two strands of DNA which differ by a single base, an insertion or deletion. Mismatch recognition proteins that cleave one strand of the mismatched DNA in the vicinity of the mismatch site may be used as cleavage agents. Nonenzymatic cleaving may also be done through photodegredation of a linker introduced through a custom oligonucleotide used in a PCR reaction.

As used herein, the term “endonuclease” refers to enzymes that cleave the phosphodiester bond within a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” An endonuclease may cut a double-stranded polynucleotide on a single strand. The methods and compositions described herein may be applied to cleavage sites generated by endonucleases. In some alternatives of the system, the system can further provide nucleic acids that encode an endonuclease, such as Cas9, TALEN, or MegaTAL, or a fusion protein including a domain of an endonuclease, for example, Cas9, TALEN, or MegaTAL, or one or more portion thereof. These examples are not meant to be limiting and other endonucleases and alternatives of the system and methods including other endonucleases and variants and modifications of these exemplary alternatives are possible without undue experimentation. All such variations and modifications are within the scope of the current teachings.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

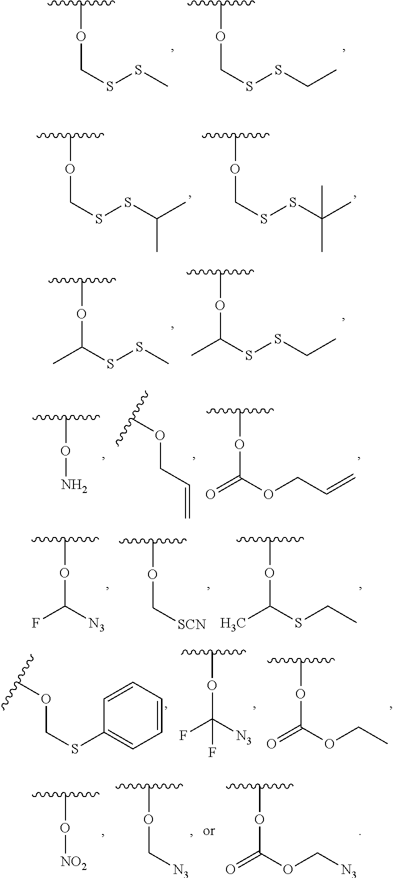

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogs include, without limitation, 7-deaza-adenine, 7-deaza-guanine, the analogs of deoxynucleotides shown herein, analogs in which a label is attached through a cleavable linker to the 5-position of cytosine or thymine or to the 7-position of deaza-adenine or deaza-guanine, and analogs in which a small chemical moiety is used to cap the OH group at the 3′-position of deoxyribose. Nucleotide analogs and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see. e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analog such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analog. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

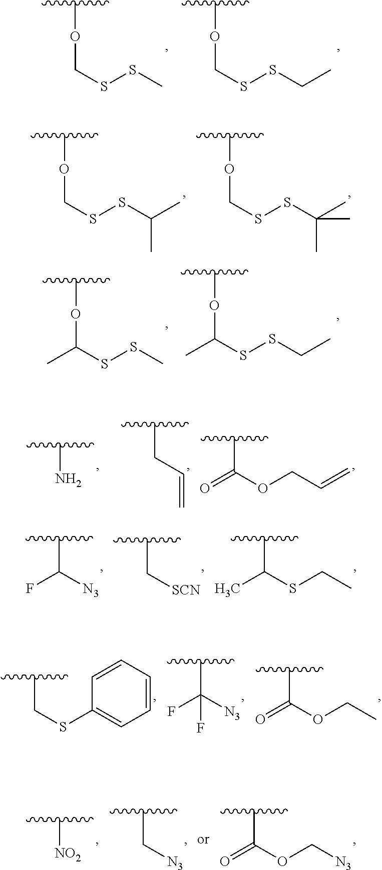

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

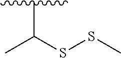 [SMILES: *C(C)SSC]

as described in U.S. Pat. No. 10,738,072, which is incorporated herein by reference for all purposes. For example, a nucleotide including a reversible terminator moiety may be represented by the formula:

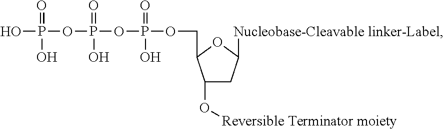

where the nucleobase is adenine or adenine analog, thymine or thymine analog, guanine or guanine analog, or cytosine or cytosine analog.

In some embodiments, a nucleic acid (e.g., an immobilized oligonucleotide) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adapters, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes.

The term “nucleobase” or “base” as used herein refers to a purine or pyrimidine compound, or a derivative thereof, that may be a constituent of nucleic acid (i.e., DNA or RNA, or a derivative thereof). In embodiments, the nucleobase is a divalent purine or pyrimidine, or derivative thereof. In embodiments, the nucleobase is a monovalent purine or pyrimidine, or derivative thereof. In embodiments, the base is a derivative of a naturally occurring DNA or RNA base (e.g., a base analog). In embodiments the base is a hybridizing base. In embodiments the base hybridizes to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analog), guanine or a derivative thereof (e.g., guanine analog), adenine or a derivative thereof (e.g., adenine analog), thymine or a derivative thereof (e.g., thymine analog), uracil or a derivative thereof (e.g., uracil analog), hypoxanthine or a derivative thereof (e.g., hypoxanthine analog), xanthine or a derivative thereof (e.g., xanthine analog), 7-methylguanine or a derivative thereof (e.g., 7-methylguanine analog), deaza-adenine or a derivative thereof (e.g., deaza-adenine analog), deaza-guanine or a derivative thereof (e.g., deaza-guanine), deaza-hypoxanthine or a derivative thereof, 5,6-dihydrouracil or a derivative thereof (e.g., 5,6-dihydrouracil analog), 5-methylcytosine or a derivative thereof (e.g., 5-methylcytosine analog), or 5-hydroxymethylcytosine or a derivative thereof (e.g., 5-hydroxymethylcytosine analog) moieties. In embodiments, the base is adenine, guanine, uracil, cytosine, thymine, hypoxanthine, xanthine, theobromine, caffeine, uric acid, or isoguanine, which may be optionally substituted or modified. In embodiments, the base is adenine, guanine, hypoxanthine, xanthine, theobromine, caffeine, uric acid, or isoguanine, which may be optionally substituted or modified.

The term “conversion” or “converted” as used herein in reference to a chemically modified nucleobase (e.g., 5-methylcytosine and 5-hydroxymethylcytosine) refers to the transformation of nucleobase to a different nucleobase. A “conversion agent” as used herein refers to a chemical or enzymatic agent that catalyzes the conversion of a nucleobase to a different nucleobase. For example, a conversion agent may catalyze the deamination of an unmodified cytosine nucleobase to a uracil nucleobase. In embodiments, a converted nucleobase is distinguishable from the modified nucleobase. For example, provided herein are methods for converting a modified cytosine (e.g., 5-methylcytosine, 5-hydroxymethylcytosine, 5-carboxylcytosine) to a uracil analog nucleobase (e.g., DHU). In embodiments, the unmodified cytosine nucleobase is converted to a uracil nucleobase (e.g., via bisulfite conversion, wherein the conversion agent is sodium bisulfite).

The term “cytosine mismatch” as used herein refers to a first nucleic acid sequence hybridized to a second nucleic acid sequence, wherein the cytosine nucleobase(s) does not form a Watson-Crick base pair with a guanine nucleobase(s). For example, a first strand having a cytosine nucleobase will form a cytosine mismatch with a second strand having a uracil nucleobase, or uracil analog, at the complementary position. In embodiments, the uracil analog is dihydrouridine (DHU).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo−)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Herminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol τ DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo−), Therminator II, Therminator III, or herminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or γ-phosphate labeled nucleotides (e.g., Therminator γ: D141A/E143A/W355A/L408 W/R460A/Q461S/K464E/D480V/R484 W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. Chem Bio Chem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by an enzyme (e.g., a DNA polymerase). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” or “template nucleic acid” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g., electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “consensus sequence” is used in accordance with its plain and ordinary meaning and refers to a theoretical representative nucleotide or amino acid sequence in which each nucleotide or amino acid is the one which occurs most frequently at that site in the different sequences which occur in nature. The phrase also refers to an actual sequence which approximates the theoretical consensus. The consensus sequence is a sequence of DNA, RNA, or protein that represents aligned, related sequences.

As used herein, the terms “solid support” and “substrate” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further include a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface including a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In some embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In some embodiments a substrate includes a magnetic material (e.g., iron, nickel, cobalt, platinum, aluminum, and the like). In certain embodiments a substrate includes a magnetic bead (e.g., DYNABEADS®, hematite, AMPure XP). Magnets can be used to purify and/or capture nucleic acids bound to certain substrates (e.g., substrates including a metal or magnetic material).

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. A microfluidic flow channel is characterized by cross-sectional dimensions less than 1000 microns. Usually at least one, and preferably all, cross-sectional dimensions are greater than 500 microns.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

The term “array” as used herein, refers to a container (e.g., a microplate, tube, or flow cell) including a plurality of features (e.g., wells). For example, an array may include a container with a plurality of wells. In embodiments, the array is a microplate. In embodiments, the array is a flow cell.

As used herein, the term “sequencing cycle” is used in accordance with its plain and ordinary meaning and refers to incorporating one or more nucleotides (e.g., nucleotide analogs) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, the term “complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As used herein, the term “hybridize” or “specifically hybridize” refers to a process where two complementary nucleic acid strands anneal to each other under appropriately stringent conditions. Hybridizations are typically and preferably conducted with oligonucleotides. The terms “annealing” and “hybridization” are used interchangeably to mean the formation of a stable duplex. Non-limiting examples of nucleic acid hybridization techniques are described in, for example, Sambrook, et al., Molecular Cloning: A Laboratory Manual (2^{nd} Edition, 1989). Those skilled in the art understand how to estimate and adjust the stringency of hybridization conditions such that sequences having at least a desired level of complementarity will stably hybridize, while those having lower complementarity will not. As used herein, the term “stringent condition” refers to condition(s) under which a polynucleotide probe or primer will hybridize preferentially to its target sequence, and to a lesser extent to, or not at all to, other sequences. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double-stranded portion of nucleic acid. The phrase “stringent hybridization conditions” refers to conditions under which a primer will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, and incubating at 42° C., or, 5×SSC, 1% SDS, and incubating at 65° C., with a wash in 0.2×SSC and 0.1% SDS at 65° C.

As used herein, “hybridizing” or “annealing” are used interchangeably in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the melting temperature (Tm) of the formed hybrid, and the G:C ratio within the nucleic acids. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. For example, hybridizing a primer (e.g., an invasion primer as described herein) to a polynucleotide strand (e.g., a strand of a double-stranded polynucleotide) includes combining the primer and the polynucleotide strand in a reaction vessel under suitable hybridization reaction conditions.

As used herein, “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, “capable of hybridizing” is used in accordance with its ordinary meaning in the art and refers to two oligonucleotides that, under suitable conditions, can form a duplex (e.g., Watson-Crick pairing) which includes a double-stranded portion of nucleic acid. Such conditions, known in the art and described herein, depend upon, for example, the nature of the nucleotide sequence, temperature, and buffer conditions. The stringency of hybridization can be influenced by various parameters, including degree of identity and/or complementarity between the polynucleotides (or any target sequences within the polynucleotides) to be hybridized; melting point of the polynucleotides and/or target sequences to be hybridized, referred to as “Tm”; parameters such as salts, buffers, pH, temperature, GC % content of the polynucleotide and primers, and/or time. Typically, hybridization is favored in lower temperatures and/or increased salt concentrations, as well as reduced concentrations of organic solvents. Some exemplary conditions suitable for hybridization include incubation of the polynucleotides to be hybridized in solutions having sodium salts, such as NaCl, sodium citrate and/or sodium phosphate. In some embodiments, hybridization or wash solutions can include about 10-75% formamide and/or about 0.01-0.7% sodium dodecyl sulfate (SDS). In some embodiments, a hybridization solution can be a stringent hybridization solution which can include any combination of 50% formamide, 5×SSC (0.75 M NaCl, 0.075 M sodium citrate), 50 mM sodium phosphate (pH 6.8), 0.1% sodium pyrophosphate, 5×Denhardt's solution, 0.1% SDS, and/or 10% dextran sulfate. In some embodiments, the hybridization or washing solution can include BSA (bovine serum albumin). In some embodiments, hybridization or washing can be conducted at a temperature range of about 20-25° C., or about 25-30° C., or about 30-35° C., or about 35-40° C., or about 40-45° C., or about 45-50° C., or about 50-55° C., or higher. In some embodiments, hybridization or washing can be conducted for a time range of about 1-10 minutes, or about 10-20 minutes, or about 20-30 minutes, or about 30-40 minutes, or about 40-50 minutes, or about 50-60 minutes, or longer. In some embodiments, hybridization or wash conditions can be conducted at a pH range of about 5-10, or about pH 6-9, or about pH 6.5-8, or about pH 6.5-7.

As used herein, the terms “denaturant” or plural “denaturants” are used in accordance with their plain and ordinary meanings and refer to an additive or condition that disrupts the base pairing between nucleotides within opposing strands of a double-stranded polynucleotide molecule. The term “denature” and its variants, when used in reference to any double-stranded polynucleotide molecule, or double-stranded polynucleotide sequence, includes any process whereby the base pairing between nucleotides within opposing strands of the double-stranded molecule, or double-stranded sequence, is disrupted. Typically, denaturation includes rendering at least some portion or region of two strands of the double-stranded polynucleotide molecule or sequence single-stranded or partially single-stranded. In some embodiments, denaturation includes separation of at least some portion or region of two strands of the double-stranded polynucleotide molecule or sequence from each other. Typically, the denatured region or portion is then capable of hybridizing to another polynucleotide molecule or sequence. Optionally, there can be “complete” or “total” denaturation of a double-stranded polynucleotide molecule or sequence. Complete denaturation conditions are, for example, conditions that would result in complete separation of a significant fraction (e.g., more than 10%, 20%, 30%, 40% or 50%) of a large plurality of strands from their extended and/or full-length complements. Typically, complete or total denaturation disrupts all of the base pairing between the nucleotides of the two strands with each other. Similarly, a nucleic acid sample is optionally considered fully denatured when more than 80% or 90% of individual molecules of the sample lack any double-strandedness (or lack any hybridization to a complementary strand).

Alternatively, the double-stranded polynucleotide molecule or sequence can be partially or incompletely denatured. A given nucleic acid molecule can be considered partially denatured when a portion of at least one strand of the nucleic acid remains hybridized to a complementary strand, while another portion is in an unhybridized state (even if it is in the presence of a complementary sequence). The unhybridized portion is optionally at least 5, 10, 15, 20, 50, or more nucleotides in length. The hybridized portion is optionally at least 5, 10, 15, 20, 50, or more nucleotides in length. Partial denaturation includes situations where some, but not all, of the nucleotides of one strand or sequence, are based paired with some nucleotides of the other strand or sequence within a double-stranded polynucleotide. In some embodiments, at least 20% but less than 100% of the nucleotide residues of one strand of the partially denatured polynucleotide (or sequence) are not base paired to nucleotide residues within the opposing strand. In embodiments, at least 50% of nucleotide residues within the double-stranded polynucleotide molecule (or double-stranded polynucleotide sequence) are in single-stranded (or unhybridized) from, but less than 20% or 10% of the residues are double-stranded.

Optionally, a nucleic acid sample can be considered to be partially denatured when a substantial fraction of individual nucleic acid molecules of the sample (e.g., above 20%, 30%, 50%, or 70%) are in a partially denatured state. Optionally less than a substantial amount of individual nucleic acid molecules in the sample are fully denatured, e.g., not more than 5%, 10%, 20%, 30% or 50% of the nucleic acid molecules in the sample. Under exemplary conditions at least 50% of the nucleic acid molecules of the sample are partly denatured, but less than 20% or 10% are fully denatured. In other situations, at least 30% of the nucleic acid molecules of the sample are partly denatured, but less than 10% or 5% are fully denatured. Similarly, a nucleic acid sample can be non-denatured when a minority of individual nucleic acid molecules in the sample are partially or completely denatured.

In an embodiment, partially denaturing conditions are achieved by maintaining the duplexes as a suitable temperature range. For example, the nucleic acid is maintained at temperature sufficiently elevated to achieve some heat-denaturation (e.g., above 45° C., 50° C., 55° C., 60° C., 65° C., or 70° C.) but not high enough to achieve complete heat-denaturation (e.g., below 95° C. or 90° C. or 85° C. or 80° C. or 75° C.). In an embodiment the nucleic acid is partially denatured using substantially isothermal conditions. Alternatively, chemical denaturation can be accomplished by contacting the double-stranded polynucleotide to be denatured with appropriate chemical denaturants, such as strong alkalis, strong acids, chaotropic agents, and the like and can include, for example, NaOH, urea, or guanidine-containing compounds. In some embodiments, partial or complete denaturation is achieved by exposure to chemical denaturants such as urea or formamide, with concentrations suitably adjusted, or using high or low pH (e.g., pH between 4-6 or 8-9). In embodiments, the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In embodiments, the first denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. In an embodiment herein, partial denaturation and/or amplification, including any one or more steps or methods described herein, can be achieved using a recombinase and/or single-stranded binding protein.

In some embodiments, complete or partial denaturation of a double-stranded polynucleotide sequence is accomplished by contacting the double-stranded polynucleotide sequence using appropriate denaturing agents. For example, the double-stranded polynucleotide can be subjected to heat-denaturation (also referred to interchangeably as thermal denaturation) by raising the temperature to a point where the desired level of denaturation is accomplished. In some embodiments, thermal denaturation of a double-stranded polynucleotide, includes adjusting the temperature to achieve complete separation of the two strands of the polynucleotide, such that 90% or greater of the strands are in single-stranded form across their entire length. In some embodiments, complete thermal denaturation of a polynucleotide molecule (or polynucleotide sequence) is accomplished by exposing the polynucleotide molecule (or sequence) to a temperature that is at least 5° C., 10° C., 15° C., 20° C., 25° C., 30° C., 50° C., or 100° C., above the calculated or predict melting temperature (Tm) of the polynucleotide molecule or sequence.

In some embodiments, complete or partial denaturation is accomplished by treating the double-stranded polynucleotide sequence to be denatured using a denaturant mixture including an SSB protein (e.g., T4 gp32 protein, T7 gene 2.5 SSB protein, or phi29 SSB protein, Thermococcus kodakarensis (KOD) SSB, Thermus thermophilus (TTH) SSB, Sulfolobus solfataricus (SSO) SSB, or Extreme Thermostable Single-Stranded DNA Binding Protein (ET-SSB)), a strand-displacing polymerase (e.g., Bst large fragment (Bst LF) polymerase, Bst 3.0 polymerase, Bst 2.0 polymerase, Bsu polymerase, SD polymerase, Vent exo− polymerase, Phi29 polymerase, or a mutant thereof), and one or more crowding agents (poly(ethylene glycol) (PEG), polyvinylpyrrolidone (PVP), bovine serum albumin (BSA), dextran, Ficoll (e.g., Ficoll 70 or Ficoll 400), glycerol, or a combination thereof). In embodiments, the crowding agent is poly(ethylene glycol) (e.g., PEG 200, PEG 600, PEG 800, PEG 2,050, PEG 4,600, PEG 6,000, PEG 8,000, PEG 10,000, PEG 20,000, or PEG 35,000), dextran sulfate, bovine pancreatic trypsin inhibitor (BPTI), ribonuclease A, lysozyme, O-lactoglobulin, hemoglobin, bovine serum albumin (BSA), or poly(sodium 4-styrene sulfonate) (PSS). In embodiments, the denaturant mixture including an SSB, a strand-displacing polymerase, and one or more crowding agents does not include a chemical denaturant (e.g., betaine, DMSO, ethylene glycol, formamide, guanidine thiocyanate, NMO, TMAC, or a mixture thereof).

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are well known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyperbranched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US20130012399), the like or combinations thereof.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein, the term “sequencing reaction mixture” is used in accordance with its plain and ordinary meaning and refers to an aqueous mixture that contains the reagents necessary to allow a nucleotide or nucleotide analog to be added to a DNA strand by a DNA polymerase. In embodiments, the sequencing reaction mixture includes modified nucleotide analogs and an enzyme in a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

The term “adapter” as used herein refers to any oligonucleotide that can be ligated to a nucleic acid molecule, thereby generating nucleic acid products that can be sequenced on a sequencing platform (e.g., an Illumina or Singular Genomics sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

As used herein, the term “hairpin adapter” refers to a polynucleotide including a double-stranded stem portion and a single-stranded hairpin loop portion. In some embodiments, an adapter is a hairpin adapter (also referred to herein as a “hairpin”). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the term “loop” is used in accordance with its plain ordinary meaning and refers to the single-stranded region of a hairpin adapter that are located between the duplexed “stem” region of the hairpin adapter. In embodiments, the hairpin loop region is between about 4 nucleotides to 150 nucleotides in length. In embodiments, the hairpin loop is at least 4, 5, 6, 7, 8, 9, 10, 15, 20, 25, 30, 35, 40, 45, 50, or more nucleotides in length. In embodiments, the hairpin loop includes 1, 2, 3, 4, 5, 6, 7, 8, 9, 10, or more T nucleotides. In embodiments, the hairpin loop may include one or more of a primer binding sequence, a barcode, a UMI sequence, or a cleavable site. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation” refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

“Synthetic” agents refer to non-naturally occurring agents, such as enzymes or nucleotides. The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

“GC bias” describes the relationship between GC content and read coverage across a genome. For example, a genomic region of a higher GC content tends to have more (or less) sequencing reads covering that region. As described herein, GC bias can be introduced during amplification of library, cluster amplification, and/or the sequencing reactions.

The term “reaction vessel” is used in accordance with its ordinary meaning in chemistry or chemical engineering, and refers to a container having an inner volume in which a reaction takes place. In embodiments, the reaction vessel may be designed to provide suitable reaction conditions such as reaction volume, reaction temperature or pressure, and stirring or agitation, which may be adjusted to ensure that the reaction proceeds with a desired, sufficient or highest efficiency for producing a product from the chemical reaction. In embodiments, the reaction vessel is a container for liquid, gas or solid. In embodiments, the reaction vessel may include an inlet, an outlet, a reservoir and the like. In embodiments, the reaction vessel is connected to a pump (e.g., vacuum pump), a controller (e.g., CPU), or a monitoring device (e.g., UV detector or spectrophotometer). In embodiments, the reaction vessel is a flow cell. In embodiments, the reaction vessel is within a sequencing device.

As used herein, a “methylated nucleotide” or a “methylated nucleobase” refers to the presence of a methyl moiety on a nucleotide base, where the methyl moiety is not present in a recognized typical nucleotide base. Similarly, a “methylated template polynucleotide” refers to a polynucleotide containing one or more methylated nucleotides. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. A methylated template polynucleotide and a methylated complement polynucleotide, both refer to a single-stranded polynucleotide including one or more methylated nucleobases. Likewise, a non-methylated (alternatively referred to as unmethylated) template polynucleotide and a non-methylated template polynucleotide both refer to a single-stranded polynucleotide that does not include one or more methylated nucleobases. In embodiments, the non-methylated complement template polynucleotide includes unmethylated cytosine nucleobases (e.g., dCTP (2′-deoxycytidine-5′-triphosphate)) and does not include any methylated cytosine nucleobases (e.g., 5-methyl dCTP). In embodiments, the methylated template polynucleotide includes one or more methylated cytosine nucleobase (e.g., 5-methyl dCTP) that complement guanine nucleobase positions in a complementary strand. In embodiments, the methylated template polynucleotide includes about 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 5% or about 1% of the cytosines of the methylated template polynucleotide are methylated cytosine nucleobase (e.g., 5-methyl dCTP).

As used herein, a “methyltransferase reagent” refers to one or more reagents that can transfer or catalyze the transfer of a methyl moiety to a compound such as a nucleotide or nucleic acid molecule. Typically a methyltransferase reagent can transfer the methyl moiety with base specificity. In embodiments, a methyltransferase reagent includes a methyltransferase (e.g., a DNA methyltransferase) and a methyltransferase ligand. In embodiments, a methyltransferase reagent includes a DNA methyltransferase and a methyltransferase ligand. Exemplary methyltransferase reagents are DNA methyltransferases, as known in the art. In some embodiments, the DNA methyltransferase is DNMT1. In embodiments, the DNA methyltransferase (e.g., DNMT1) methylates cytosine residues in hemimethylated DNA in the sequence of 5′ . . . CG . . . 3′. Additional useful methylating agents include DNMT3a and DNMT3b which are mammalian methyl transferases. Additional useful methylating agents include DRM2, MET1, and CMT3 which are plant methyl transferases. Additional useful methylating agents include Dam which is a bacterial methyl transferase. In embodiments, the methyltransferase reagent is DNMT1, M.SssI, DNMT, or a homolog or mutant thereof. In embodiments, the methyltransferase reagent is DNMT3a, DNMT3b, DRM2, MET1, and CMT3, or a homolog or mutant thereof. Methyltransferase reagents may also include a molecule capable of providing a methyl moiety (e.g., a source of methyl moieties). In embodiments, a methyltransferase ligand includes a molecule capable of providing a methyl moiety. For example, a molecule capable of providing a methyl moiety may include S-adenosylmethionine (SAM), or an analog thereof (e.g., S-8-aza-adenosylmethionine (8-aza-SAM), S-2-aminopurinylmethionine (2AP-SAM), S-2,6-diaminopurinylmethionine (DAPSAM)). In embodiments, the molecule is a S-adenosyl methionine (SAM) labeled analog (e.g., a SAM moiety covalently linked to a fluorescent dye). Methyltransferases (e.g., DNMT1) are typically used with a source of methyl moieties and may be used with or without cofactors known to those of skill in the art. DNMT1 works in vitro at 95% efficiency without a cofactor, however, DNMT1 may be used with a cofactor such as UHRF1 as described in Bashtrykov et al. (J Biol Chem. 2014 Feb. 14; 289(7):4106-15). In embodiments, a chelating agent, such as EDTA, is used after the extension step to chelate ions (e.g., magnesium ions) in order for the methylation transfer to occur. In embodiments, the chelating agent is iminodisuccinic acid (IDS), polyaspartic acid, ethylenediamine-N, N′-disuccinic acid (EDDS), Methylglycinediacetic acid, aminopolycarboxylate-based chelates, tetrasodium salt or N-diacetic acid.

As used herein, the term “CpG island” refers to a genomic DNA region that contains a high percentage of CpG sites relative to the average genomic CpG incidence (per same species, per same individual, or per subpopulation (e.g., strain, ethnic subpopulation, or the like). Various parameters and definitions for CpG islands exist; for example, in some embodiments, CpG islands are defined as having a GC percentage that is greater than 50% and with an observed/expected CpG ratio that is greater than 60% (Gardiner-Garden et al. (1987) J Mol. Biol. 196:261-282; Baylin et al. (2006) Nat. Rev. Cancer 6:107-116; Irizarry et al. (2009) Nat. Genetics 41:178-186; each herein incorporated by reference in its entirety). In some embodiments, CpG islands may have a GC content >55% and observed CpG/expected CpG of 0.65 (Takai et al. (2007) PNAS 99:3740-3745; herein incorporated by reference in its entirety). Various parameters also exist regarding the length of CpG islands. As used herein, CpG islands may be less than 100 bp; 100-200 bp, 200-300 bp, 300-500 bp, 500-750 bp; 750-1000 bp; 1000 or more bp in length. In some embodiments, CpG islands show altered methylation patterns (e.g., altered 5hmC patterns) relative to controls (e.g., altered 5hmC methylation in cancer subjects relative to subjects without cancer; tissue-specific altered 5hmC patterns; altered 5hmC patterns in biological samples from subjects with a neoplasia or tumor relative to subjects without a neoplasia or tumor. In some embodiments, altered methylation involves increased incidence of 5hmC. In some embodiments, altered methylation involves decreased incidence of 5hmC.

As used herein, the term “CpG shore” or “CpG island shore” refers to a genomic region external to a CpG island that is or that has potential to have altered methylation (e.g., 5hmC) patterns (see, e.g., Irizarry et al. (2009) Nat. Genetics 41:178-186; herein incorporated by reference in its entirety). CpG island shores may show altered methylation (e.g., 5hmC) patterns relative to controls (e.g., altered 5hmC in cancer subjects relative to subjects without cancer; tissue-specific altered 5hmC patterns; altered 5hmC in biological samples from subjects with neoplasia or tumor relative to subjects without neoplasia or tumor. In some embodiments, altered methylation involves increased incidence of 5hmC. In some embodiments, altered methylation involves decreased incidence of 5hmC. CpG island shores may be located in various regions relative to CpG islands (see, e.g., Irizarry et al. (2009) Nat. Genetics 41; 178-186; herein incorporated by reference in its entirety). Accordingly, in some embodiments, CpG island shores are located less than 100 bp; 100-250 bp; 250-500 bp; 500-1000 bp; 1000-1500 bp; 1500-2000 bp; 2000-3000 bp; 3000 bp or more away from a CpG island.

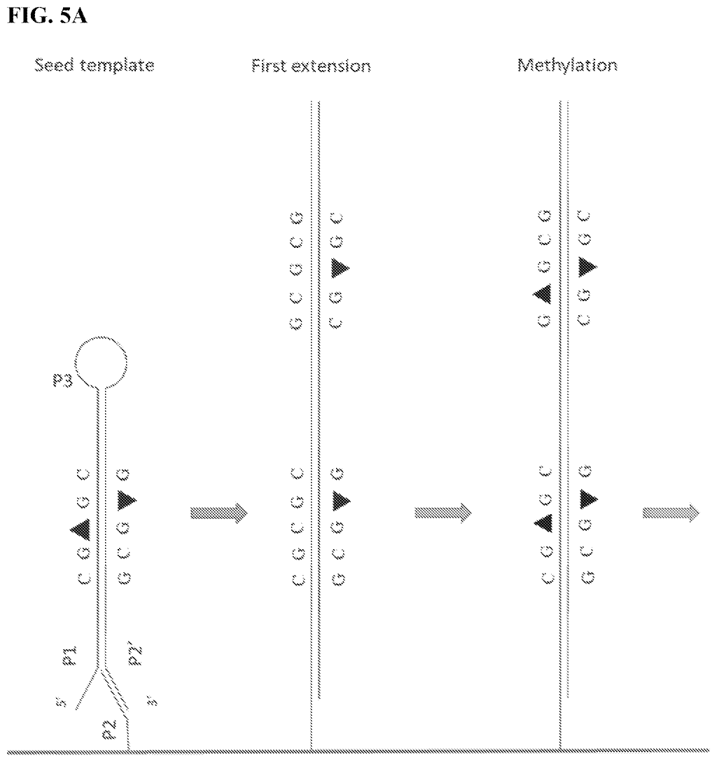

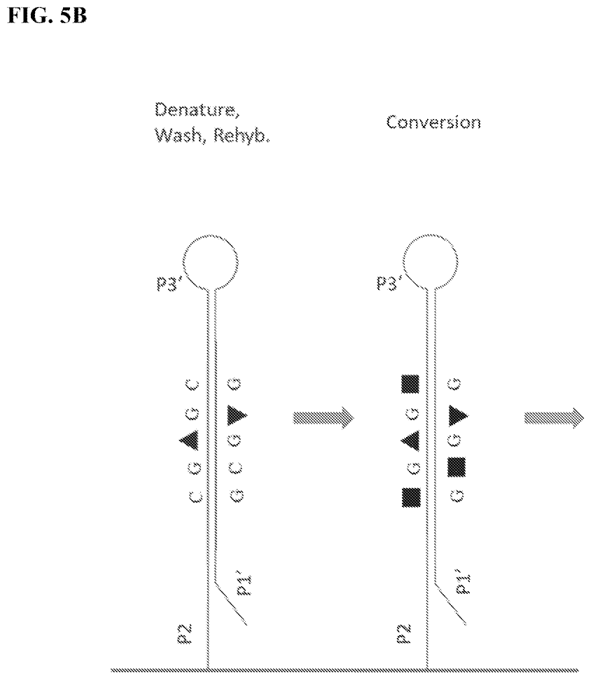

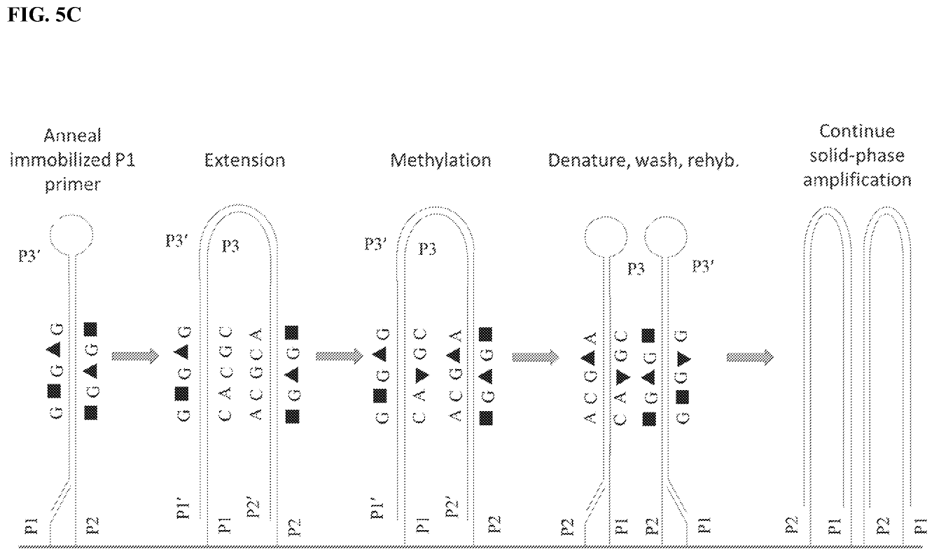

As used herein, the “methylation state” or “methylation pattern” of a target nucleic acid molecule refers to the presence of absence of one or more methylated nucleotide bases in a target nucleic acid molecule. For example, a target nucleic acid molecule containing a methylated cytosine is considered methylated (i.e., the methylation state of the target nucleic acid molecule is methylated). A target nucleic acid molecule that does not contain any methylated nucleotides is considered unmethylated. Similarly, the methylation state of a nucleotide locus in a target nucleic acid molecule refers to the presence or absence of a methylated nucleotide at a particular locus in the target nucleic acid molecule. For example, the methylation state of a cytosine at the 7^{th} nucleotide in a target nucleic acid molecule is methylated when the nucleotide present at the 7^{th} nucleotide in the target nucleic acid molecule is 5-methylcytosine. Similarly, the methylation state of a cytosine at the 7^{th} nucleotide in a target nucleic acid molecule is unmethylated when the nucleotide present at the 7^{th} nucleotide in the target nucleic acid molecule is cytosine (and not 5-methylcytosine). In embodiments, the methylated cytosine nucleobase on the methylated template polynucleotide is proximal to the methylated sites on the methylated complement template polynucleotide. In embodiments, the methylated cytosine nucleobase on the methylated template polynucleotide is complementary to the guanine nucleobase on the methylated complement template polynucleotide, wherein the guanine nucleobase is adjacent to a methylated cytosine (see for example, FIGS. 5A-5B).

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Methods

In an aspect is provided a method of generating an immobilized methylated complement template polynucleotide, the method including: i) hybridizing a methylated template polynucleotide to a first immobilized primer at a first temperature, wherein the first immobilized primer is attached to a solid support; ii) extending the first immobilized primer with a polymerase to generate an immobilized non-methylated complement template polynucleotide hybridized to the methylated template polynucleotide; and iii) contacting the immobilized non-methylated complement template polynucleotide with a DNA methyltransferase reagent to generate an immobilized methylated complement template polynucleotide, wherein the methylated complement template polynucleotide includes one or more methylated cytosine nucleobases and one or more non-methylated cytosine nucleobases. In embodiments, the method further includes iv) denaturing the immobilized methylated complement template polynucleotide from the methylated template polynucleotide at a second temperature, wherein the second temperature is higher than the first temperature. In embodiments, the method further includes v) repeating steps i)-iv), thereby generating a plurality of immobilized methylated polynucleotides.

In an aspect is provided a method of generating a plurality of immobilized methylated polynucleotides, the method including: i) hybridizing a methylated template polynucleotide to a first immobilized primer at a first temperature, wherein the first immobilized primer is attached to a solid support; ii) extending the first immobilized primer with a polymerase to generate an immobilized non-methylated complement template polynucleotide hybridized to the methylated template polynucleotide; iii) contacting the non-methylated complement template polynucleotide with a methyltransferase reagent to generate a methylated complement template polynucleotide, wherein the methylated complement template polynucleotide includes one or more methylated cytosine nucleobases and one or more non-methylated cytosine nucleobases; iv) denaturing the methylated complement template polynucleotide from the methylated template polynucleotide at a second temperature, wherein the second temperature is higher than the first temperature; and v) repeating steps i)-iv), thereby generating a plurality of immobilized methylated polynucleotides.

In embodiments, step iv) includes contacting the methylated complement template polynucleotide with a chemical denaturant. In embodiments, the methods further includes vi) removing the chemical denaturant and hybridizing the methylated complement template polynucleotide to a second immobilized primer at the first temperature, wherein the second immobilized primer is attached to the solid support; and extending the second immobilized primer with a polymerase to generate a complement of the methylated complement template polynucleotide hybridized to the methylated complement template polynucleotide. In embodiments, the method further includes vii) contacting the complement of the methylated complement template polynucleotide with a methyltransferase reagent to generate a methylated template polynucleotide hybridized to the methylated complement template polynucleotide, wherein the methylated template polynucleotide includes one or more methylated cytosine nucleobases, and contacting the methylated complement template polynucleotide with a conversion agent to convert the one or more non-methylated cytosine nucleobases to uracil nucleobases, thereby generating a uracil-containing polynucleotide including one or more uracil nucleobases. In embodiments, the method further includes vii) contacting the immobilized complement of the methylated complement template polynucleotide with a DNA methyltransferase reagent to generate an immobilized methylated template polynucleotide hybridized to the immobilized methylated complement template polynucleotide, wherein the immobilized methylated template polynucleotide includes one or more methylated cytosine nucleobases and one or more non-methylated cytosine nucleobases, and contacting the immobilized methylated complement template polynucleotide with a conversion agent to convert the one or more non-methylated cytosine nucleobases to uracil nucleobases, thereby generating an immobilized uracil-containing polynucleotide including one or more uracil nucleobases. In embodiments, the method further includes repeating steps (i) to (vii), thereby amplifying the template polynucleotide.

In an aspect is provided a method of generating a methylated complement template polynucleotide including: (a) annealing a methylated template polynucleotide to a first immobilized primer on a solid support at a first temperature, wherein the first immobilized primer is complementary to a sequence of the template polynucleotide; (b) extending the first primer with a polymerase to generate a non-methylated complement template polynucleotide; and (c) contacting the non-methylated complement template polynucleotide with a methyltransferase reagent to generate a methylated complement template polynucleotide hybridized to the methylated template polynucleotide, wherein the methylated complement template polynucleotide includes one or more methylated cytosine nucleobases and one or more non-methylated cytosine nucleobases. In embodiments, the method further includes denaturing the methylated complement template polynucleotide from the methylated template polynucleotide at a second temperature, wherein the second temperature is higher than the first temperature.

In embodiments, the method includes contacting the methylated complement template polynucleotide with a chemical denaturant. In embodiments, the method further includes (d) removing the denaturant and annealing the methylated complement template polynucleotide to a second immobilized primer on the solid support at the first temperature, wherein the second immobilized primer is complementary to a sequence of the methylated complement template polynucleotide; and extending the second immobilized primer with the polymerase to generate a complement of the methylated complement template polynucleotide. In embodiments, the method further includes (e) contacting the complement of the methylated complement template polynucleotide with a methyltransferase reagent to generate a methylated template polynucleotide hybridized to the methylated complement template polynucleotide, wherein the methylated template polynucleotide includes one or more methylated cytosine nucleobases, and contacting the methylated complement template polynucleotide with a conversion agent to convert the one or more non-methylated cytosine nucleobases to uracil nucleobases, thereby generating a uracil-containing strand including one or more uracil nucleobases.

In embodiments, contacting the immobilized non-methylated complement template polynucleotide with the DNA methyltransferase reagent occurs at a third temperature, wherein the third temperature is lower than said first temperature.

In embodiments, contacting the non-methylated complement template polynucleotide with the methyltransferase occurs at a third temperature, wherein the third temperature is lower than the first temperature.

In embodiments, the method further includes repeating steps (a) to (e), thereby amplifying the template polynucleotide.

In embodiments, the methods described herein generate amplification products (e.g., amplicons) immobilized on a solid support thereby forming arrays comprised of colonies. In embodiments, the amplification product is provided in a clustered array. In embodiments, the clustered array includes a plurality of double-stranded amplification products localized to discrete sites on a solid support. In embodiments, the solid support is a bead. In embodiments, the solid support is substantially planar. In embodiments, the solid support is contained within a flow cell. In embodiments, amplifying includes a plurality of cycles of strand denaturation, primer hybridization, and primer extension. In embodiments, amplifying includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In embodiments, the method further includes contacting the methylated complement template polynucleotide with a conversion agent thereby converting the one or more non-methylated cytosine nucleobases to one or more uracil nucleobases and generating a uracil-containing strand. In embodiments, the method further includes contacting the methylated complement template polynucleotide with a second conversion agent thereby converting the one or more methylated cytosine nucleobases to one or more 5-carboxylcytosine (5caC) nucleobases.

In embodiments, the method further includes contacting the methylated complement template polynucleotide with a conversion agent thereby converting the one or more non-methylated cytosine nucleobases to one or more uracil nucleobases and generating a uracil-containing polynucleotide. In embodiments, the method further includes contacting the methylated complement template polynucleotide with a second conversion agent thereby converting the one or more methylated cytosine nucleobases to one or more 5-carboxylcytosine (5caC) nucleobases. In embodiments, the method further includes annealing a primer to the uracil-containing polynucleotide and extending the primer hybridized to the uracil-containing polynucleotide with a polymerase to generate an amplification product.

In embodiments, the method further includes contacting the methylated complement template polynucleotide with a conversion agent thereby converting the one or more methylated cytosine nucleobases to one or more uracil or uracil analog nucleobases and generating a uracil-containing polynucleotide.

In embodiments, the method further includes contacting the methylated complement template polynucleotide with a conversion agent thereby converting the one or more methylated cytosine nucleobases to one or more uracil or uracil analog nucleobases and generating a uracil-containing strand (alternatively referred to as a deaminated strand). In embodiments, the uracil analog is dihydrouridine (DHU).

In embodiments, the method further includes applying oxygen to the solid support prior to removing the chemical denaturant.

In embodiments, the method further includes applying air to the solid support prior to removing the chemical denaturant.

In embodiments, the method includes amplifying the uracil-containing strand to generate one or more amplification products. In embodiments, the method includes annealing a primer to the uracil-containing strand and extending with a polymerase to generate an amplification product.

In embodiments, the method includes amplifying the uracil-containing strand with bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR, or combinations of the methods. In embodiments, generating an amplification product includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, generating an amplification product includes a bridge polymerase chain reaction amplification. In embodiments, generating an amplification product includes a thermal bridge polymerase chain reaction (t-bPCR) amplification. In embodiments, generating an amplification product includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

In embodiments, the methylated template polynucleotide includes one or more 5-methylcytosine (5mC) or 5-hydroxymethyl cytosine (5hmC) nucleobases. In embodiments, the methylated template polynucleotide includes one or more 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC) nucleobases. In embodiments, the methylated template polynucleotide includes one or more 5-methylcytosine (5mC) nucleobases. In embodiments, the methylated template polynucleotide includes one or more 5-hydroxymethyl cytosine (5hmC) nucleobases. In embodiments, the methylated template polynucleotide includes one or more 5-formylcytosine (5fC) nucleobases. In embodiments, the methylated template polynucleotide includes one or more 5-carboxylcytosine (5caC) nucleobases. In embodiments, the methylated template polynucleotide includes one or more 0-glucosyl-5-hydroxymethylcytosine (5gmC) nucleobases.

In embodiments, the method further includes prior to step a) contacting the solid support with a sample including a methylated template polynucleotide (e.g., a methylated template polynucleotide as described herein). In embodiments, the method further includes prior to step i) contacting the solid support with a sample including a methylated template polynucleotide (e.g., a methylated template polynucleotide as described herein). In embodiments, one or more initial steps (i.e., the first steps) are different from the remaining steps of the method. For example, the initial denaturation step is maintained at different conditions from the remaining denaturation steps. For example, the initial methylation step is maintained at different conditions from the remaining methylation steps. In embodiments, the initial extension step is maintained at different conditions from the remaining extension steps. In embodiments, the initial extension includes an initial extension solution that is different from the remaining extension solutions. In embodiments, the initial extension solution includes MgCl₂, strand-displacing (SD) polymerase, dNTPs, and betaine.

In embodiments, the first primer is covalently attached to the solid support via a first linker and the second primer is covalently attached to the solid support via a second linker. The linker tethering the polynucleotides may be any linker capable of localizing nucleic acids to arrays. The linkers may be the same, or the linkers may be different. Solid-supported molecular arrays have been generated previously in a variety of ways, for example, the attachment of biomolecules (e.g., proteins and nucleic acids) to a variety of substrates (e.g., glass, plastics, or metals) underpins modern microarray and biosensor technologies employed for genotyping, gene expression analysis and biological detection. Silica-based substrates are often employed as supports on which molecular arrays are constructed, and functionalized silanes are commonly used to modify glass to permit a click-chemistry enabled linker to tether the biomolecule. In embodiments, the solid support includes a polymer coating wherein the immobilized primers are covalently linked to the polymer. In embodiments, the immobilized primers include primer binding sequences (i.e., regions of complementarity for a primer) which enable specific annealing of primers when the template polynucleotides are in used in the solid-phase amplification reaction. In embodiments, the immobilized primers are methylated. In embodiments, the immobilized primers are non-methylated. In embodiments, the first primer and the second primer are methylated. In embodiments, the first primer and the second primer are non-methylated. In embodiments, the first primer is methylated and the second primer is non-methylated. In embodiments, the first primer is non-methylated and the second primer is methylated.

In embodiments, the methylated cytosine nucleobases include 5-methylcytosine (5mC) or 5-hydroxymethyl cytosine (5hmC). In embodiments, the methylated cytosine nucleobase is a 5-methylcytosine (5mC) nucleobase or a 5-hydroxymethyl cytosine (5hmC) nucleobase.

In embodiments, extending the first primer occurs at the first temperature. In embodiments, extending the first primer occurs at the second temperature. In embodiments, extending the first primer occurs at a temperature between the first temperature and the second temperature.

In embodiments, the method further includes contacting the methylated complement template polynucleotide with a denaturant at a temperature between the second temperature and the first temperature. In embodiments, extending the first primer occurs at a third temperature, T₁₂, wherein T₁₂ is between the first temperature and the second temperature. In embodiments, T₁₂ is a transition temperature, i.e., the reaction is not maintained at that temperature for a significant amount of time (e.g., less than 1 min, less than 10 seconds). In embodiments, the increase from the first temperature to the second temperature occurs at a variable rate (i.e., or ΔT/t). In embodiments, the increase from the first temperature to the second temperature occurs at a controlled rate (i.e., or ΔT/Δt). For example, temperature may be increased at a rate of about 0.1° C./s to about 5° C./s. In embodiments, temperature may be increased at a rate of about 0.2° C./s. In embodiments, temperature may be increased at a rate of about 0.3° C./s. In embodiments, temperature may be increased at a rate of about 0.4° C./s. In embodiments, temperature may be increased at a rate of about 0.5° C./s. In embodiments, temperature may be increased at a rate of about 0.5° C./s. In embodiments, temperature may be increased at a rate of about 0.75° C./s. In embodiments, temperature may be increased at a rate of about 1° C./s. In embodiments, temperature may be increased at a rate of about 1.25° C./s. In embodiments, temperature may be increased at a rate of about 1.5° C./s. In embodiments, temperature may be increased at a rate of about 1.75° C./s. In embodiments, temperature may be increased at a rate of about 2° C./s. In embodiments, temperature may be increased at a rate of about 2.25° C./s. In embodiments, temperature may be increased at a rate of about 2.5° C./s. In embodiments, temperature may be increased at a rate of about 2.75° C./s. In embodiments, temperature may be increased at a rate of about 3° C./s. In embodiments, temperature may be increased at a rate of about 3.25° C./s. In embodiments, temperature may be increased at a rate of about 3.5° C./s. In embodiments, temperature may be increased at a rate of about 3.75° C./s. In embodiments, temperature may be increased at a rate of about 4° C./s. In embodiments, temperature may be increased at a rate of about 4.25° C./s. In embodiments, temperature may be increased at a rate of about 4.5° C./s. In embodiments, temperature may be increased at a rate of about 4.75° C./s.

In embodiments, the first temperature increases to the second temperature at a fixed rate. In embodiments, the second temperature decreases to the first temperature at a fixed rate. In embodiments, the decrease from the second temperature to the first temperature occurs at a controlled rate. In embodiments, the increase from the first temperature to the second temperature occurs at a controlled rate. In embodiments, the first temperature increases to the second temperature at a controlled rate. In embodiments, the second temperature decreases to the first temperature at a controlled rate. In embodiments, the decrease from the second temperature to the first temperature occurs at a variable rate. In embodiments, the increase from the first temperature to the second temperature occurs at a variable rate.

In embodiments, the first temperature is about 25° C. to about 45° C., or about 40° C. to about 45° C. In embodiments, the first temperature is a temperature between about 25° C. to about 45° C. In embodiments, the first temperature is a temperature between about 40° C. to about 45° C. In embodiments, the first temperature ranges from about 25° C. to about 45° C. In embodiments, the first temperature ranges from about 40° C. to about 45° C. In embodiments, the first temperature is about 40° C. to about 45° C. In embodiments, the first temperature is about 30° C., 31° C., 32° C., 33° C., 34° C., 35° C., 36° C., 37° C., 38° C., 39° C., 40° C., 41° C., 42° C., 43° C., 44° C., or about 45° C. In embodiments, the first temperature is about 40° C., 41° C., 42° C., 43° C., 44° C., or about 45° C. In embodiments, the first temperature is 40° C. In embodiments, the first temperature is 41° C. In embodiments, the first temperature is 42° C. In embodiments, the first temperature is 43° C. In embodiments, the first temperature is 44° C. In embodiments, the first temperature is 45° C.

In embodiments, the second temperature is about 45° C. to about 70° C., or about 55° C. to about 62° C. In embodiments, the second temperature is a temperature between about 45° C. to about 70° C. In embodiments, the second temperature is a temperature between about 55° C. to about 62° C. In embodiments, the second temperature ranges from about 45° C. to about 70° C. In embodiments, the second temperature ranges from about 55° C. to about 62° C. In embodiments, the second temperature is about 55° C. to about 65° C. In embodiments, the second temperature is about 55° C., 56° C., 57° C., 58° C., 59° C., 60° C., 61° C., 62° C., 63° C., 64° C., or about 65° C. In embodiments, the first temperature and the second temperature differ by about 5° C. In embodiments, the first temperature and the second temperature differ by about 10° C. In embodiments, the first temperature and the second temperature differ by about 15° C. In embodiments, the first temperature and the second temperature differ by about 20° C. In embodiments, the first temperature and the second temperature differ by about 25° C. In embodiments, the first temperature and the second temperature differ by 5° C. In embodiments, the first temperature and the second temperature differ by 10° C. In embodiments, the first temperature and the second temperature differ by 15° C. In embodiments, the first temperature and the second temperature differ by 20° C. In embodiments, the first temperature and the second temperature differ by 25° C. In embodiments, the first temperature and the second temperature differ by no greater than 25° C. In embodiments, the first temperature and the second temperature differ by no greater than 20° C. In embodiments, the first temperature and the second temperature differ by no greater than 19° C. In embodiments, the first temperature and the second temperature differ by no greater than 18° C. In embodiments, the first temperature and the second temperature differ 12-18° C.

In embodiments, the method includes cycling between a first temperature and a second temperature, wherein the difference between the first temperature and the second temperature is no greater than 20° C. In embodiments, the method includes cycling between a first temperature and a second temperature, wherein the difference between the first temperature and the second temperature is no greater than 19° C. In embodiments, the method includes cycling between a first temperature and a second temperature, wherein the difference between the first temperature and the second temperature is no greater than 18° C. In embodiments, the method includes cycling between a first temperature and a second temperature, wherein the difference between the first temperature and the second temperature is no greater than 16° C. In embodiments, the method includes cycling between a first temperature and a second temperature, wherein the difference between the first temperature and the second temperature is 10° C. to 18° C. In embodiments, the method includes cycling between a first temperature and a second temperature, wherein the difference between the first temperature and the second temperature is 12° C. to 18° C. In embodiments, the method includes cycling between a first temperature and a second temperature, wherein the difference between the first temperature and the second temperature is 18° C. In embodiments, the method includes cycling between a first temperature and a second temperature, wherein the difference between the first temperature and the second temperature is 19° C. In embodiments, the method includes cycling between a first temperature and a second temperature, wherein the difference between the first temperature and the second temperature is 1° C. to 19° C. In embodiments, the method includes cycling between a first temperature and a second temperature, wherein the difference between the first temperature and the second temperature is 10° C. to 19° C. In embodiments, the method includes cycling between a first temperature and a second temperature, wherein the difference between the first temperature and the second temperature is 12° C. to 18° C.

In embodiments, the first temperature and the second temperature differ by about 5° C. to about 15° C. In embodiments, the first temperature and the second temperature differ by no greater than 20° C. Additional embodiments and examples of methods of extending and amplifying at variable temperatures are described in, e.g., International Application No. PCT/US2022/034761, which is hereby incorporated by reference in its entirety.

In embodiments, the third temperature is about 30° C. to about 40° C. In embodiments, the third temperature is about 30° C., 31° C., 32° C., 33° C., 34° C., 35° C., 36° C., 37° C., 38° C., 39° C., or about 40° C. In embodiments, the third temperature is about 30° C. In embodiments, the third temperature is about 31° C. In embodiments, the third temperature is about 32° C. In embodiments, the third temperature is about 33° C. In embodiments, the third temperature is about 34° C. In embodiments, the third temperature is about 35° C. In embodiments, the third temperature is about 36° C. In embodiments, the third temperature is about 37° C. In embodiments, the third temperature is about 38° C. In embodiments, the third temperature is about 39° C. In embodiments, the third temperature is about 40° C.

In embodiments, extending the first primer includes incorporating one or more nucleotides (e.g., dNTPs) using a polymerase (e.g., Bst large fragment (Bst LF) polymerase, Bst2.0 polymerase, Bsu polymerase, SD polymerase, Vent exo− polymerase, Phi29 polymerase, or a mutant thereof). In embodiments, the polymerase is a strand-displacing polymerase. In embodiments, the strand-displacing polymerase is Bst large fragment (Bst LF) polymerase, Bst 3.0 polymerase, Bst2.0 polymerase, Bsu polymerase, SD polymerase, Vent exo− polymerase, Phi29 polymerase, or a mutant thereof. In embodiments, the polymerase is Bst DNA Polymerase, Vent (exo−) DNA Polymerase, Pfu DNA polymerase, Taq polymerase, Phusion High-Fidelity DNA Polymerase, Q5 High-Fidelity DNA Polymerase, or mutant of any one of the foregoing. In embodiments, the polymerase is Bst DNA Polymerase, Vent (exo−) DNA Polymerase, Phusion High-Fidelity DNA Polymerase, or Q5 High-Fidelity DNA Polymerase.

In embodiments, the method includes contacting the methylated complement template polynucleotide with a chemical denaturant at a temperature between the second temperature and the first temperature. In embodiments, contacting the methylated complement template polynucleotide with a denaturant at a fourth temperature, T₂₁, wherein T₂₁ is a temperature between the second temperature and the second temperature. In embodiments, T₂₁ is a transition temperature, i.e., the reaction is not maintained at that temperature for a significant amount of time (e.g., less than 1 min, less than 10 seconds). In embodiments, the decrease from the second temperature to the first temperature occurs at a controlled rate (i.e., or ΔT/Δt). For example, temperature may be decreased at a rate of about 0.1° C./s to about 5° C./s. In embodiments, temperature may be decreased at a rate of about 0.2° C./s. In embodiments, temperature may be decreased at a rate of about 0.3° C./s. In embodiments, temperature may be decreased at a rate of about 0.4° C./s. In embodiments, temperature may be decreased at a rate of about 0.5° C./s. In embodiments, temperature may be decreased at a rate of about 0.5° C./s. In embodiments, temperature may be decreased at a rate of about 0.75° C./s. In embodiments, temperature may be decreased at a rate of about 1° C./s. In embodiments, temperature may be decreased at a rate of about 1.25° C./s. In embodiments, temperature may be decreased at a rate of about 1.5° C./s. In embodiments, temperature may be decreased at a rate of about 1.75° C./s. In embodiments, temperature may be decreased at a rate of about 2° C./s. In embodiments, temperature may be decreased at a rate of about 2.25° C./s. In embodiments, temperature may be decreased at a rate of about 2.5° C./s. In embodiments, temperature may be decreased at a rate of about 2.75° C./s. In embodiments, temperature may be decreased at a rate of about 3° C./s. In embodiments, temperature may be decreased at a rate of about 3.25° C./s. In embodiments, temperature may be decreased at a rate of about 3.5° C./s. In embodiments, temperature may be decreased at a rate of about 3.75° C./s. In embodiments, temperature may be decreased at a rate of about 4° C./s. In embodiments, temperature may be decreased at a rate of about 4.25° C./s. In embodiments, temperature may be decreased at a rate of about 4.5° C./s. In embodiments, temperature may be decreased at a rate of about 4.75° C./s.

In embodiments, the annealing is performed in the presence of an annealing solution. In embodiments, the annealing solution includes a buffered solution including salts (e.g., NaCl or KCl), a surfactant (e.g., Triton X-100 or Tween), and a chelator. In embodiments, the annealing solution has a pH of about 8.0, 8.2, 8.4, 8.6, 8.8, or 9.0. In embodiments, the annealing solution includes NaCl, Tris (e.g., pH 8.0), Triton X-100, and a chelator (e.g., EDTA). In embodiments, the annealing solution includes NaCl, Tris (e.g., pH 8.5), Triton X-100, and a chelator (e.g., EDTA). In embodiments, the annealing solution includes NaCl, Tris (e.g., pH 8.8), Triton X-100, and a chelator (e.g., EDTA).

In embodiments, the extending is performed in the presence of an extension solution. In embodiments, the extension solution includes a buffered solution including salts (e.g., NaCl or KCl), a surfactant (e.g., Triton X-100 or Tween-20), and a chelator. In embodiments, the extension solution includes nucleotides and a polymerase (e.g., a polymerase as described herein). In embodiments, the extension solution includes about 2, about 3, about 4, about 5, about 6, about 7, about 8, about 9, about 10, about 11, about 12, about 13, about 14, or about 15 mM Mg2+. In embodiments, the extension solution includes a dNTP mixture including dATP, dCTP, dGTP and dTTP (for DNA amplification) or dATP, dCTP, dGTP and dUTP (for RNA amplification). In embodiments, the extension solution has a pH of about 8.0, 8.2, 8.4, 8.6, 8.8, or 9.0. In embodiments, the extension solution includes Tris-HCl (e.g., pH 8.0), salt (e.g, NaCl or KCl), MgSO4, a surfactant (e.g., Tween-20), dNTPs, BstLF, betaine, and/or DMSO. In embodiments, the extension solution includes bicine (e.g., pH 8.5), salt (e.g, NaCl or KCl), MgSO4, a surfactant (e.g., Tween-20), dNTPs, BstLF, betaine, and/or DMSO.

In embodiments, step (c) is performed in the presence of a methylation solution. In embodiments, step (iii) is performed in the presence of a methylation solution. In embodiments, the methylation solution includes a buffered solution including a methyltransferase and a methyl donor compound. In embodiments, the methylation solution includes a DNA methyltransferase. In embodiments, the methylation solution includes a methylation ligand. In embodiments, the methylation solution includes a DNA methyltransferase and a methylation ligand. In embodiments, the methylation solution includes a source of methyl groups (e.g., S-adenosyl-1-methionine (SAM)). In embodiments, the methyltransferase reagent is DNMT1. In embodiments, the DNA methyltransferase (e.g., DNMT1) methylates cytosine residues in hemimethylated DNA in the sequence of 5′ . . . CG . . . 3′. In embodiments, a methyltransferase reagent includes a methyltransferase (e.g., a DNA methyltransferase) and a methyltransferase ligand. In embodiments, a methyltransferase reagent includes a DNA methyltransferase and a methyltransferase ligand. In embodiments, a methyltransferase ligand includes a molecule capable of providing a methyl moiety (e.g., S-adenosyl-1-methionine (SAM)). In embodiments, the methyltransferase reagent is DNMT1, M.SssI, DNMT, or a homolog or mutant thereof. In embodiments, the methyltransferase reagent is DNMT3a, DNMT3b, DRM2, MET1, and CMT3, or a homolog or mutant thereof. In embodiments, the methyltransferase reagent includes DNMT1. In embodiments, the methyltransferase reagent includes DNMT1, M.SssI, DNMT, or a homolog or mutant thereof. In embodiments, the methyltransferase reagent includes DNMT3a, DNMT3b, DRM2, MET1, and CMT3, or a homolog or mutant thereof. In embodiments, the methylation solution, also referred to as a methylation reagent, includes a buffered solution of DNMT1 and SAM. In embodiments, the DNA methyltransferase reagent includes DNMT1. In embodiments, the DNA methyltransferase reagent includes DNMT1, M.SssI, DNMT, or a homolog or mutant thereof. In embodiments, the DNA methyltransferase reagent includes DNMT3a, DNMT3b, DRM2, MET1, and CMT3, or a homolog or mutant thereof. In embodiments, the methylation solution, also referred to as a methylation reagent, is a buffered solution of DNMT1 and SAM. In embodiments, the DNA methyltransferase is DNMT1. In embodiments, the DNA methyltransferase is DNMT1, M.SssI, DNMT, or a homolog or mutant thereof. In embodiments, the DNA methyltransferase is DNMT3a, DNMT3b, DRM2, MET1, and CMT3, or a homolog or mutant thereof.

In embodiments, the method further includes contacting the amplification product with a chemical denaturant thereby separating the first strand and the amplification product; annealing a second immobilized primer to the second strand; and repeating step (c). In embodiments, the method further includes contacting the amplification product with a chemical denaturant thereby separating the first strand and the amplification product; annealing a second non-methylated immobilized primer to the second strand; and repeating step (c). In embodiments, the method further includes contacting the amplification product with a chemical denaturant thereby separating the first strand and the amplification product; annealing a second methylated immobilized primer to the second strand; and repeating step (c). In embodiments, the method further includes contacting the amplification product with a chemical denaturant thereby separating the first polynucleotide and the amplification product; annealing a second immobilized primer to the second polynucleotide; and repeating step (iii).

In embodiments, the chemical denaturant includes formamide, ethylene glycol, sodium hydroxide, or a mixture thereof. In embodiments, the chemical denaturant includes formamide, ethylene glycol, or sodium hydroxide. In embodiments, the chemical denaturant includes formamide. In embodiments, the chemical denaturant is formamide. In embodiments, the chemical denaturant is formamide, and no other chemical denaturants are present. In embodiments, the chemical denaturant is pure formamide. In embodiments, the chemical denaturant is pure formamide, and no other chemical denaturants are present. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO). In embodiments, the denaturant is betaine. In embodiments, the denaturant is dimethyl sulfoxide (DMSO). In embodiments, the denaturant is ethylene glycol. In embodiments, the denaturant is formamide. In embodiments, the denaturant is glycerol. In embodiments, the denaturant is guanidine thiocyanate. In embodiments, the denaturant is 4-methylmorpholine 4-oxide (NMO). In embodiments, the denaturant is betaine, dimethyl sulfoxide (DMSO), ethylene glycol, formamide, glycerol, guanidine thiocyanate, or 4-methylmorpholine 4-oxide (NMO). In embodiments, the denaturant includes an organic diol (e.g., 1,3 propanediol, 1,2-butanediol, 1,3-butanediol, 1,6-hexanediol, 1,2-hexanediol, 2-methyl-2,4-pentanediol), for example 0.01M to about 2.5M organic diol. In embodiments, the denaturant is ethylene glycol, polyethylene glycol, 1,2-propanediol, dimethyl sulfoxide (DMSO), glycerol, formamide, 7-deaza-dGTP, acetamide, betaine or tetramethylammonium chloride (TMAC). The addition of chemical denaturants such as betaine, DMSO, and formamide can be helpful when amplifying GC-rich templates and templates that form strong secondary structures, which can cause DNA polymerases to stall. For example, DMSO and formamide independently are understood to interfere with the formation of hydrogen bonds between the two DNA strands.

In embodiments, the chemical denaturant includes formamide, ethylene glycol, or sodium hydroxide. In embodiments, the chemical denaturant includes formamide. In embodiments, the chemical denaturant is pure formamide (i.e., 100% formamide). In embodiments, the chemical denaturant includes formamide, ethylene glycol, sodium hydroxide, or a mixture thereof. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO). In embodiments, the chemical denaturant is sodium hydroxide.

In embodiments, the method further includes contacting the amplification product with a methyltransferase reagent to generate a methylated amplification product.

In embodiments, the denaturant includes additives such as ethylene glycol, polyethylene glycol, 1,2-propanediol, dimethyl sulfoxide (DMSO), glycerol, formamide, 7-deaza-dGTP, acetamide, betaine, or tetramethylammonium chloride (TMAC). In embodiments, the denaturant includes ethylene glycol. Ethylene glycol destabilizes duplex DNA, and the melting point of DNA decreases by about 0.5° C. for each 1% (vol/vol) concentration of glycerol or ethylene glycol. The denaturation temperature may be finely tuned according to the concentration of denaturants. In embodiments, the denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. In embodiments, the denaturant is a buffered solution including about 50% to about 100% formamide. In embodiments, the denaturant is a buffered solution including about 100% formamide. In embodiments, the denaturant is a buffered solution including 100% formamide.

In embodiments, the denaturant, the extension solution, and/or the annealing solution includes one or more crowding agents. In embodiments, the crowding agent is poly(ethylene glycol) (e.g., PEG 200, PEG 600, PEG 800, PEG 2,050, PEG 4,600, PEG 6,000, PEG 8,000, PEG 10,000, PEG 20,000, or PEG 35,000). In embodiments, PEG is present in the denaturant at a concentration of 1% to 25%. In embodiments, PEG is present in the denaturant at a concentration of about 1%, about 5%, about 10%, about 15%, about 20%, or about 25%.

In embodiments, each transition between a different solution includes applying oxygen (e.g., air) to the solid support. In embodiments, the method further includes applying oxygen to the solid support prior to removing the denaturant. In embodiments, the method further includes applying air to the solid support prior to removing the denaturant. In embodiments, one or more pulses of air are provided to the solid support. In embodiments, each transition between a different solution includes flushing out the solution. In embodiments, each transition between a different solution includes flushing out the solution and applying oxygen (e.g., air) to the solid support.

In embodiments, removing the denaturant includes application of a wash solution. In embodiments, the wash solution is at a pH from pH 8 to pH 9. In embodiments, the wash solution includes a chelator. In embodiments, the wash solution includes a surfactant. In embodiments, at least one washing step can be conducted after any of steps described herein. In embodiments, the wash includes Tris-HCl, pH 8.5, containing SDS, EDTA, and NaCl. The wash solution can include SSC (e.g., at any concentration of about 1-5×) and a detergent (e.g., Tween-20 or Triton X-100).

In embodiments, the metal chelating agent (i.e., a chelator) in the wash solution includes EDTA (ethylenediaminetetraacetic acid), EGTA (ethylene glycol tetraacetic acid), HEDTA (hydroxyethylethylenediaminetriacetic acid), DPTA (diethylene triamine pentaacetic acid), NTA (N,N-bis(carboxymethyl)glycine), citrate anhydrous, sodium citrate, calcium citrate, ammonium citrate, ammonium bicitrate, citric acid, potassium citrate, or magnesium citrate. In some embodiments, the wash solution includes a chelating agent at a concentration of about 0.01-50 mM, or about 0.1-20 mM, or about 0.2-10 mM.

In some embodiments, the salt in the wash solution includes NaCl, KCl, NH₂SO₄ or potassium glutamate. In some embodiments, the detergent includes an ionic detergent such as SDS (sodium dodecyl sulfate). The wash solution can include a monovalent salt at a concentration of about 25-500 mM, or about 50-250 mM, or about 100-200 mM. In embodiments, the detergent in the wash solution includes a non-ionic detergent such as Triton X-100, Tween 20, Tween 80 or Nonidet P-40. In embodiments, the detergent includes a zwitterionic detergent such as CHAPS (3-[(3-cholamidopropyl)dimethylammonio]-1-propanesulfonate) or N-Dodecyl-N,N-dimethyl-3-ammonio-1-propanesulfate (DetX). In some embodiments, the detergent includes LDS (lithium dodecyl sulfate), sodium taurodeoxycholate, sodium taurocholate, sodium glycocholate, sodium deoxycholate or sodium cholate. In some embodiments, the detergent is included in the wash solution at a concentration of about 0.01-0.05%, or about 0.05-0.1%, or about 0.1-0.15%, or about 0.15-0.2%, or about 0.2-0.25%. In embodiments, the wash solution includes SSC (e.g., at any concentration of about 1-5×) and a detergent (e.g., Tween-20 or Triton X-100).

In embodiments, the solid support includes a plurality of immobilized oligonucleotides (e.g., immobilized primers, such as immobilized forward and immobilized reverse primers, or immobilized first and immobilized second primers) attached to the solid support via a linker. Additional examples of immobilized oligonucleotides include, for example, an immobilized non-methylated complement template polynucleotide, an immobilized methylated complement template polynucleotide, a plurality of immobilized methylated polynucleotides, an immobilized uracil-containing polynucleotide, an immobilized complement of the immobilized methylated complement template polynucleotide, or a complement thereof. In embodiments, the methylated template polynucleotide and methylated complement template polynucleotide are covalently attached to the solid support (also referred to herein as the immobilized methylated template polynucleotide and immobilized methylated complement template polynucleotide). In embodiments, the non-methylated complement template polynucleotide is covalently attached to the solid support (also referred to herein as the immobilized non-methylated complement template polynucleotide). In embodiments, the methylated template polynucleotide is covalently attached to the solid support (also referred to herein as the immobilized methylated template polynucleotide). In embodiments, the methylated complement template polynucleotide is covalently attached to the solid support (also referred to herein as the immobilized methylated complement template polynucleotide). In embodiments, the plurality of methylated polynucleotides is covalently attached to the solid support (also referred to herein as the plurality of immobilized methylated polynucleotides). In embodiments, the uracil-containing polynucleotide is covalently attached to the solid support (also referred to herein as the immobilized uracil-containing polynucleotide). In embodiments, the complement of the immobilized methylated complement template polynucleotide is covalently attached to the solid support (also referred to herein as the immobilized complement of the immobilized methylated complement template polynucleotide). In embodiments, the forward or first primers are covalently attached to the solid support (also referred to herein as the immobilized forward primers or immobilized first primers). In embodiments, the reverse or second primers are covalently attached to the solid support (also referred to herein as the immobilized reverse primers or immobilized second primers). In embodiments, the amplification primer (e.g., first or second amplification primer) is covalently attached to the solid support (also referred to herein as the immobilized amplification primer). In embodiments, the sequencing primer is covalently attached to the solid support (also referred to herein as the immobilized sequencing primer).

In embodiments, the solid support includes immobilized polynucleotides (e.g., immobilized polynucleotides, such as immobilized methylated template polynucleotides) attached to the solid support via a non-covalent attachment (e.g., by hybridization to an oligonucleotide, wherein the oligonucleotide is covalently attached to the solid support). In embodiments, the methylated template polynucleotide is non-covalently attached to the solid support (also referred to herein as the immobilized methylated template polynucleotide). In embodiments, the forward or first primers are non-covalently attached to the solid support. In embodiments, the reverse or second primers are non-covalently attached to the solid support. In embodiments, the amplification primer is non-covalently attached to the solid support. In embodiments, the sequencing primer is non-covalently attached to the solid support.

In embodiments, the 5′ end of the template and complement template polynucleotides contains a functional group that serves to tether the template and complement template polynucleotides to the solid support (e.g., a bioconjugate linker). Non-limiting examples of covalent attachment (e.g. for the attachment of the immobilized oligonucleotides described herein) include amine-modified polynucleotides reacting with epoxy or isothiocyanate groups on the solid support, succinylated polynucleotides reacting with aminophenyl or aminopropyl functional groups on the solid support, dibenzocycloctyne-modified polynucleotides reacting with azide functional groups on the solid support (or vice versa), trans-cyclooctyne-modified polynucleotides reacting with tetrazine or methyl tetrazine groups on the solid support (or vice versa), disulfide modified polynucleotides reacting with mercapto-functional groups on the solid support, amine-functionalized polynucleotides reacting with carboxylic acid groups on the core via 1-ethyl-3-(3-dimethylaminopropyl)-carbodiimide hydrochloride (EDC) chemistry, thiol-modified polynucleotides attaching to a solid support via a disulfide bond or maleimide linkage, alkyne-modified polynucleotides attaching to a solid support via copper-catalyzed click reactions to azide functional groups on the solid support, and acrydite-modified polynucleotides polymerizing with free acrylic acid monomers on the solid support to form polyacrylamide or reacting with thiol groups on the solid support. In embodiments, the primer is attached to the solid support polymer through electrostatic binding. For example, the negatively charged phosphate backbone of the primer may be bound electrostatically to positively charged monomers in the solid support.

In embodiments, the solid support (alternatively referred to as a substrate) includes a silica surface including a polymer coating. In embodiments, the solid support includes a glass surface including a polymer coating. In embodiments, the substrate is silica or quartz, such as a microscope slide, having a surface that is uniformly silanized. This may be accomplished using conventional protocols, such as those described in Beattie et al (1995), Molecular Biotechnology, 4: 213. Such a surface is readily treated to permit end-attachment of oligonucleotides (e.g., forward and reverse primers) prior to amplification. In embodiments the substrate surface further includes a polymer coating, which contains functional groups capable of immobilizing primers. In some embodiments, the substrate includes a patterned surface suitable for immobilization of primers in an ordered pattern. A patterned surface refers to an arrangement of different regions in or on an exposed layer of a substrate. For example, one or more of the regions can be features where one or more primers are present. The features can be separated by interstitial regions where capture primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the primers are randomly distributed upon the substrate. In some embodiments, the primers are distributed on a patterned surface. In embodiments, the solid support includes a particle having a surface that includes a polymer coating. In embodiments, the immobilized primers are immobilized to the polymer coated particle. In embodiments, the polymer coated particles are themselves immobilized on a planar substrate. In embodiments, the solid support includes a discrete particle. In embodiments, the solid support includes a nanoparticle.

In embodiments, the solid support is a multiwell container including a plurality of wells, each well including a polymer as described herein. In embodiments, the polymer includes polymerized units of polyacrylamide (AAm), poly-N-isopropylacrylamide, poly N-isopropylpolyacrylamide, sulfobetaine acrylate (SBA), carboxybetaine acrylate (CBA), phosphorylcholine acrylate (PCA), sulfobetaine methacrylate (SBMA), carboxybetaine methacrylate (CBMA), phosphorylcholine methacrylate (PCMA), polyethylene glycol acrylate, methacrylate, polyethylene glycol (PEG)-thiol/PEG-acrylate, acrylamide/N,N′-bis(acryloyl)cystamine (BACy), PEG/polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, glicydyl methacrylate (GMA), glicydyl methacrylate (GMA) azide, hydroxyethylmethacrylate (HEMA), hydroxyethylacrylate (HEA), hydroxypropylmethacrylate (HPMA), polyethylene glycol methacrylate (PEGMA), polyethylene glycol acrylate (PEGA), isocyanatoethyl methacrylate (IEM), or a copolymer thereof. In embodiments, the polymer includes polymerized units of polyethylene glycol methacrylate (PEGMA) and glicydyl methacrylate (GMA). In embodiments, the polymer includes polymerized units of polyethylene glycol methacrylate (PEGMA) and isocyanatoethyl methacrylate (IEM). In embodiments, the polymer includes polymerized units of glicydyl methacrylate azide (GMA azide) and polyethylene glycol methacrylate (PEGMA). In embodiments, the polymer includes a plurality of oligonucleotides (e.g., immobilized primers as described herein) covalently attached to the polymer. In embodiments, the polymer coating includes polymerized units of polyacrylamide (AAm), glicydyl methacrylate (GMA), glicydyl methacrylate (GMA) azide, polyethylene glycol methacrylate (PEGMA), polyethylene glycol methacrylate (PEGMA), isocyanatoethyl methacrylate (IEM), or a copolymer thereof. In embodiments, the polymer layer includes the particle polymer includes polymerized units of a) polyethylene glycol methacrylate (PEGMA) and glicydyl methacrylate (GMA), b) polyethylene glycol methacrylate (PEGMA) and isocyanatoethyl methacrylate (IEM), or c) polyethylene glycol methacrylate (PEGMA) and glicydyl methacrylate (GMA) azide.

In embodiments, the solid support includes a polymer layer. In embodiments, the polymer layer includes polymerized units of alkoxysilyl methacrylate, alkoxysilyl acrylate, alkoxysilyl methylacrylamide, alkoxysilyl methylacrylamide, or a copolymer thereof. In embodiments, the polymer layer includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

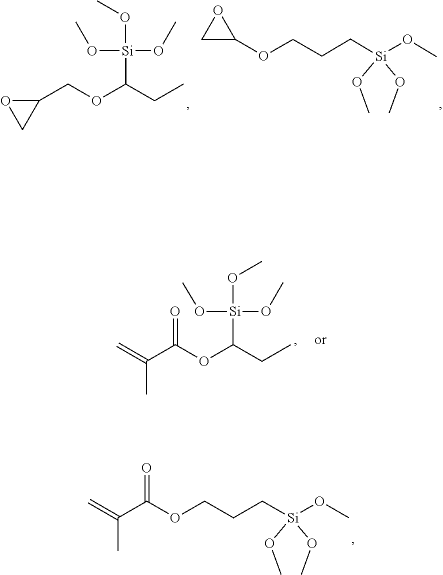

or a copolymer thereof.

In embodiments, the solid support includes a photoresist, alternatively referred to herein as a resist. A “resist” as used herein is used in accordance with its ordinary meaning in the art of lithography and refers to a polymer matrix (e.g., a polymer network). In embodiments, the photoresist is a silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, or a organically modified ceramic polymer resist. In embodiments, the photoresist is a silsesquioxane resist. In embodiments, the photoresist is an epoxy-based polymer resist. In embodiments, the photoresist is a poly(vinylpyrrolidone-vinyl acrylic acid) copolymer resist. In embodiments, the photoresist is an Off-stoichiometry thiol-enes (OSTE) resist. In embodiments, the photoresist is an amorphous fluoropolymer resist. In embodiments, the photoresist is a crystalline fluoropolymer resist. In embodiments, the photoresist is a polysiloxane resist. In embodiments, the photoresist is an organically modified ceramic polymer resist. In embodiments, the photoresist includes polymerized alkoxysilyl methacrylate polymers and metal oxides (e.g., SiO₂, ZrO, MgO, Al₂O₃, TiO₂ or Ta₂O₅). In embodiments, the photoresist includes polymerized alkoxysilyl acrylate polymers and metal oxides (e.g., SiO₂, ZrO, MgO, Al₂O₃, TiO₂ or Ta₂O₅). In embodiments, the photoresist includes metal atoms, such as Si, Zr, Mg, Al, Ti or Ta atoms.

In embodiments, the solid support is subjected to lithographic patterning methods (e.g., nanolithographic to microlithographic patterning). In embodiments, prior to contacting the solid support with a plurality of particles, the solid support is subjected to lithographic patterning methods (e.g., nanolithographic to microlithographic patterning). Typically, features smaller than 10 micrometers are considered microlithographic, and features smaller than 100 nanometers are considered nanolithographic. Lithographic techniques make use of masks or templates to transfer patterns over a large area simultaneously. A powerful microfabrication technique is photolithography, i.e. the lithography using a UV light source and a photosensitive material as resist. As the name suggests, the photoresist (alternatively referred to as a resist) is an active material layer that can be patterned by selective exposure and must “resist” chemical/physical attach of the underlying substrate. In embodiments, the resist is a crosslinked polymer matrix.

In embodiments, the solid support includes a resist (e.g., a nanoimprint lithography (NIL) resist). Nanoimprint resists can include thermal curable materials (e.g., thermoplastic polymers), and/or UV-curable polymers. In embodiments, the solid support is generated by pressing a transparent mold possessing the pattern of interest (e.g., the pattern of wells) into photo-curable liquid film, followed by solidifying the liquid materials via a UV light irradiation. Typical UV-curable resists have low viscosity, low surface tension, and suitable adhesion to the glass substrate. For example, the solid support surface, but not the surface of the wells, is coated in an organically modified ceramic polymer (ORMOCER®, registered trademark of Fraunhofer-Gesellschaft zur Förderung der angewandten Forschung e. V. in Germany). Organically modified ceramics contain organic side chains attached to an inorganic siloxane backbone. Several ORMOCER® polymers are now provided under names such as “Ormocore”, “Ormoclad” and “Ormocomp” by Micro Resist Technology GmbH. In embodiments, the solid support includes a resist as described in Haas et al Volume 351, Issues 1-2, 30 Aug. 1999, Pages 198-203, US 2015/0079351A1, US 2008/0000373, or US 2010/0160478, each of which is incorporated herein by reference. In embodiments, the solid support surface, and the surface of the wells, is coated in an organically modified ceramic polymer (ORMOCER®, registered trademark of Fraunhofer-Gesellschaft zur Förderung der angewandten Forschung e. V. in Germany). In embodiments, the resist (e.g., the organically modified ceramic polymer) is not removed prior to particle deposition. In embodiments, the wells are within the resist polymer and not the solid support. In embodiments, the solid support includes a plurality of wells (e.g., a billion or more wells). In embodiments, the wells (e.g., each well) is separated by about 0.1 μm to about 5.0 μm. In embodiments, the wells (e.g., each well) is separated by about 0.2 μm to about 2.0 μm. In embodiments, the wells (e.g., each well) is separated by about 0.5 μm to about 1.5 μm. In embodiments, the wells of the solid support are all the same size. In embodiments, one or more wells are different sizes (e.g., one population of wells are 1.0 μm in diameter, and a second population are 0.5 μm in diameter). In embodiments, the solid support is a glass slide about 75 mm by about 25 mm.

In embodiments, density of wells on the solid support may be tuned. For example, in embodiments, the multiwell container includes a density of at least about 100 wells per mm², about 1,000 wells per mm², about 0.1 million wells per mm², about 1 million wells per mm², about 2 million wells per mm², about 5 million wells per mm², about 10 million wells per mm², about 50 million wells per mm², or more. In embodiments, the multiwell container includes no more than about 50 million wells per mm², about 10 million wells per mm², about 5 million wells per mm², about 2 million wells per mm², about 1 million wells per mm², about 0.1 million wells per mm², about 1,000 wells per mm², about 100 wells per mm², or less. In embodiments, the solid support includes about 500, 1,000, 2,500, 5,000, or about 25,000 wells per mm². In embodiments, the solid support includes about 1×10⁶ to about 1×10¹² wells. In embodiments, the solid support includes about 1×10⁷ to about 1×10¹² wells.

In embodiments, the solid support includes about 1×10⁸ to about 1×10¹² wells. In embodiments, the solid support includes about 1×10⁶ to about 1×10⁹ wells. In embodiments, the solid support includes about 1×10⁹ to about 1×10¹⁰ wells. In embodiments, the solid support includes about 1×10⁷ to about 1×10⁹ wells. In embodiments, the solid support includes about 1×10⁸ to about 1×10⁸ wells. In embodiments, the solid support includes about 1×10⁶ to about 1×10⁸ wells. In embodiments, the solid support includes about 1×10⁶, 1×10⁷, 1×10⁸, 1×10⁹, 1×10¹⁰, 1×10¹¹, 1×10¹², 5×10¹², or more wells. In embodiments, the solid support includes about 1.8×10⁹, 3.7×10⁹, 9.4×10⁹, 1.9×10¹⁰, or about 9.4×10¹⁰ wells. In embodiments, the solid support includes about 1×10⁶ or more wells. In embodiments, the solid support includes about 1×10⁷ or more wells. In embodiments, the solid support includes about 1×10⁸ or more wells. In embodiments, the solid support includes about 1×10⁹ or more wells. In embodiments, the solid support includes about 1×10¹⁰ or more wells. In embodiments, the solid support includes about 1×10¹¹ or more wells. In embodiments, the solid support includes about 1×10¹² or more wells. In embodiments, the solid support is a glass slide. In embodiments, the solid support is a about 75 mm by about 25 mm. In embodiments, the solid support includes one, two, three, or four channels.

In embodiments, the particle is a functionalized particle including a particle core (e.g., a silica particle core) and a polymer shell, wherein the polymer shell is covalently attached to the particle core and includes a plurality of polymerized units of shell monomers and one or more shell monomers includes an oligonucleotide moiety covalently linked to the shell monomer (e.g., an immobilized primer, as described herein). In embodiments, the oligonucleotide moiety is covalently linked to the shell monomer via a bioconjugate linker.

In embodiments, each particle includes a plurality of oligonucleotide moieties covalently attached to the particle via a polymeric bioconjugate linker. In embodiments, the polymeric bioconjugate linker is a polymer (i.e., a molecule including structurally unique repeating units) including one or more reacted bioconjugate reactive moieties that formed a bioconjugate linker. For example, a bioconjugate linker is illustrated in Scheme 1. In embodiments, the polymeric bioconjugate linker is a polymer including a subunit of formula Ia, Ib, II, or III as described in U.S. Pat. No. 11,236,387, which is incorporated herein by reference in its entirety and for all purposes.

In embodiments, the oligonucleotide moiety includes a DBCO bioconjugate reactive moiety that reacts with an azide bioconjugate reactive moiety on the polymer and forms a bioconjugate linker that covalently links the oligonucleotide moiety to the polymer, for example according to the following scheme:

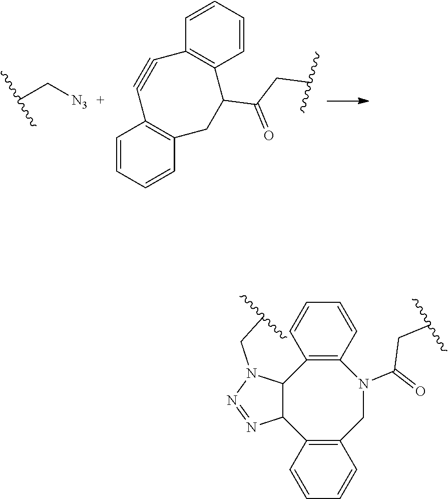 [SMILES: *CN=[N+]=[N-] ; *CC(=O)C1Cc2ccccc2C#Cc2ccccc21 ; *CC(=O)N1Cc2ccccc2C2N=NN(C*)C2c2ccccc21]

Scheme 1. An example mechanism of the bioconjugate covalent linker formed by reacting a DBCO containing oligonucleotide with a particle containing an azide moiety, wherein the

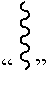

refers to the attachment point to the oligonucleotide moiety and the polymer, respectively.

In embodiments, the particle has a polymer shell surrounding the particle core. In embodiments, the polymer shell includes polymerized units of polyacrylamide (AAm), poly-N-isopropylacrylamide, poly N-isopropylpolyacrylamide, sulfobetaine acrylate (SBA), carboxybetaine acrylate (CBA), phosphorylcholine acrylate (PCA), sulfobetaine methacrylate (SBMA), carboxybetaine methacrylate (CBMA), phosphorylcholine methacrylate (PCMA), polyethylene glycol acrylate, methacrylate, polyethylene glycol (PEG)-thiol/PEG-acrylate, acrylamide/N,N′-bis(acryloyl)cystamine (BACy), PEG/polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, glicydyl methacrylate (GMA), glicydyl methacrylate (GMA) azide, hydroxyethylmethacrylate (HEMA), hydroxyethylacrylate (HEA), hydroxypropylmethacrylate (HPMA), polyethylene glycol methacrylate (PEGMA), polyethylene glycol acrylate (PEGA), isocyanatoethyl methacrylate (IEM), or a copolymer thereof. In embodiments, the polymer shell includes polymerized units of polyethylene glycol methacrylate (PEGMA) and glicydyl methacrylate (GMA). In embodiments, the polymer shell includes polymerized units of polyethylene glycol methacrylate (PEGMA) and isocyanatoethyl methacrylate (IEM). In embodiments, the polymer shell includes polymerized units of glicydyl methacrylate azide (GMA azide) and polyethylene glycol methacrylate (PEGMA).

In embodiments, the polymer shell includes polymerized units of 3-azido-2-hydroxypropyl methacrylate, 2-azido-3-hydroxypropyl methacrylate, 2-(((2-azidoethoxy)carbonyl)amino)ethyl methacrylate, 3-azido-2-hydroxypropyl acrylate, 2-azido-3-hydroxypropyl acrylate, or 2-(((2-azidoethoxy)carbonyl)amino)ethyl acrylate. In embodiments, the polymer shell includes polymerized units of 3-azido-2-hydroxypropyl methacrylate, 2-azido-3-hydroxypropyl methacrylate, or 2-(((2-azidoethoxy)carbonyl)amino)ethyl methacrylate. In embodiments, the polymer shell includes polymerized units of 3-azido-2-hydroxypropyl methacrylate. In embodiments, the polymer shell includes polymerized units of 3-azido-2-hydroxypropyl methacrylate 2-azido-3-hydroxypropyl methacrylate. In embodiments, the polymer shell includes polymerized units of 3-azido-2-hydroxypropyl methacrylate 2-(((2-azidoethoxy)carbonyl)amino)ethyl methacrylate. In embodiments, the polymer shell includes polymerized units of a) polyethylene glycol methacrylate (PEGMA) and glicydyl methacrylate (GMA), b) polyethylene glycol methacrylate (PEGMA) and isocyanatoethyl methacrylate (IEM), or c) polyethylene glycol methacrylate (PEGMA) and glicydyl methacrylate (GMA) azide. In embodiments, the polymer shell is permeable to a polymerase.

In embodiments, the polymer (e.g., the polymer coating or the polymer shell) includes polymerized units of glicydyl methacrylate azide (GMA azide) and polyethylene glycol methacrylate (PEGMA) in the ratio of 1:1. In embodiments, the ratio of GMA azide to PEGMA is 1:2. In embodiments, the ratio of GMA azide to PEGMA is 1:3. In embodiments, the ratio of GMA azide to PEGMA is 1:4. In embodiments, the ratio of GMA azide to PEGMA is 1:5. In embodiments, the ratio of GMA azide to PEGMA is 1:6. In embodiments, the ratio of GMA azide to PEGMA is 1:7. In embodiments, the ratio of GMA azide to PEGMA is 1:8.

The polymer may be polymerized from a mixture of functionalized and non-functionalized monomers, such that at least some functionalized monomers that provide attachment points (e.g., azide moieties) for primers (e.g., DBCO-containing oligonucleotide primers) are spaced from one another by one or more monomers lacking such attachment points (e.g., PEG or AAm). The frequency of monomer units attached to primers within a polymer can be adjusted by changing the concentration of the corresponding functionalized monomer in the mixture of monomers. In embodiments, monomer units of the polymer that are attached to a polynucleotide primer (referred to herein as oligonucleotide moieties) are separated by, on average, about or at least about 1, 2, 3, 4, 5, 6, 7, 8, 9, 10, 15, 20, 25, 30, 40, 50, or more monomer units that are not attached to a primer, referred to herein as (ng). In embodiments, monomer units of the polymer that are attached to a polynucleotide primer are separated by, on average, about or at least about 4 to 8 monomer units that are not attached to a primer. In embodiments, monomer units of the polymer that are attached to a polynucleotide primer are separated by, on average, about or at least about 6, 7, or 8 monomer units that are not attached to a primer. In embodiments, primer-attached monomers are separated by, on average, about 1-50, 2-40, 3-30, 4-25, or 5-20 monomers not attached to primers. In embodiments, monomer units of the polymer that are attached to a polynucleotide primer are separated by 3 monomer units that are not attached to a primer (aka 3 ng). In embodiments, monomer units of the polymer that are attached to a polynucleotide primer are separated by 6 ng. In embodiments, monomer units the polymer that are attached to a polynucleotide primer are separated by 9 ng. The mixture can include monomers with different functional groups (e.g., azides, alkynes, DBCO, etc.) as described herein.

In embodiments, the average longest dimension of the particle is from about 100 nm to about 3000 nm. In embodiments, the average longest dimension of the particle is from about 200 nm to about 2900 nm. In embodiments, the average longest dimension of the particle is from about 300 nm to about 2800 nm. In embodiments, the average longest dimension of the particle is from about 400 nm to about 2700 nm. In embodiments, the average longest dimension of the particle is from about 500 nm to about 2600 nm. In embodiments, the average longest dimension of the particle is from about 600 nm to about 2500 nm. In embodiments, the average longest dimension of the particle is from about 700 nm to about 2400 nm. In embodiments, the average longest dimension of the particle is from about 800 nm to about 2300 nm. In embodiments, the average longest dimension of the particle is from about 900 nm to about 2200 nm. In embodiments, the average longest dimension of the particle is from about 1000 nm to about 2100 nm. In embodiments, the average longest dimension of the particle is from about 900 nm to about 2000 nm. In embodiments, the average longest dimension of the particle is from about 150 nm to about 600 nm. In some embodiments, the average longest dimension of the particle is from about 350 nm to about 600 nm. In some embodiments, the average longest dimension of the particle is from about 400 nm to about 500 nm. In some embodiments, the average longest dimension of the particle is about 500 nm. In some embodiments, the average longest dimension of the particle is about 400 nm. In some embodiments, the average longest dimension of the particle is about 400 nm, 450 nm, 500 nm, or 550 nm. In some embodiments, the average longest dimension of the particle is about 410 nm, 420 nm, 430 nm, 440 nm or 450 nm. In some embodiments, the average longest dimension of the particle is about 460 nm, 470 nm, 480 nm, 490 nm or 500 nm. In embodiments, the average longest dimension of the particle is at least, about, or at most 100, 200, 300, 400, 500, 600, 700, 800, 900, 1000 nm or a number or a range between any two of these values. In embodiments, the particle shell diameter is at least, about, or at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4., 4.5, 4.6, 4.7, 4.8, 4.9, 5.0 μm or a number or a range between any two of these values. In embodiments, the core diameter is about 150-700 nanometers, and/or the shell diameter is about 0.25-5 μm (microns).

In embodiments, the average longest dimension of the nanoparticle is from about 100 nm to about 400 nm. In embodiments, the average longest dimension of the nanoparticle is about 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 595 nm, or 600 nm. In embodiments, the average longest dimension of the nanoparticle is from about 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775 nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875 nm, 880 nm, 885 nm, 890 nm, 895 nm, 900 nm, 905 nm, 910 nm, 915 nm, 920 nm, 925 nm, 930 nm, 935 nm, 940 nm, 945 nm, 950 nm, 955 nm, 960 nm, 965 nm, 970 nm, 975 nm, 980 nm, 985 nm, 990 nm, 995 nm or about 1000 nm. In embodiments, the average longest dimension of the nanoparticle is less than about 1000 nm. In embodiments, the average longest dimension of the nanoparticle is less than about 900 nm. In embodiments, the average longest dimension of the nanoparticle is less than about 800 nm. In embodiments, the average longest dimension of the nanoparticle is less than about 700 nm. In embodiments, the average longest dimension of the nanoparticle is less than about 600 nm. In embodiments, the average longest dimension of the nanoparticle is less than about 500 nm. In embodiments, the average longest dimension of the nanoparticle is less than about 400 nm. In embodiments, the average longest dimension of the nanoparticle is less than about 300 nm. In embodiments, the average longest dimension of the nanoparticle is less than about 200 nm. In embodiments, the average longest dimension of the nanoparticle is less than about 100 nm. In embodiments, the average longest dimension of the nanoparticle is 400 nm without the particle shell.

In embodiments, the solid support includes a plurality of bioconjugate reactive moieties. In embodiments, a bioconjugate reactive moiety includes an amine moiety, aldehyde moiety, alkyne moiety, azide moiety, carboxylic acid moiety, dibenzocyclooctyne (DBCO) moiety, norbornene moiety, tetrazine moiety, epoxy moiety, isocyanate moiety, furan moiety, maleimide moiety, thiol moiety, or transcyclooctene (TCO) moiety. In embodiments, the particle includes a plurality of azide moieties, alkyne moieties, dibenzocyclooctyne (DBCO) moieties, norbornene moieties, epoxy moieties, or isocyanate moieties. In some embodiments, the solid support includes a plurality of oligonucleotide moieties (e.g., ssDNA moieties) covalently attached via a bioconjugate linker to the solid support (e.g., via a polymeric bioconjugate linker or via the polymer shell). The bioconjugate linker is the product of a reaction between the two bioconjugate group (e.g. click chemistry group). In embodiments, each of the plurality of bioconjugate reactive moieties includes an amine moiety, aldehyde moiety, alkyne moiety, azide moiety, carboxylic acid moiety, dibenzocyclooctyne (DBCO) moiety, norbornene moiety, tetrazine moiety, epoxy moiety, isocyanate moiety, furan moiety, maleimide moiety, thiol moiety, or transcyclooctene (TCO) moiety. In embodiments, each of the plurality of bioconjugate reactive moieties include an amine moiety, azide moiety, dibenzocyclooctyne (DBCO) moiety, epoxy moiety, or isocyanate moiety. In embodiments, each of the plurality of bioconjugate reactive moieties include an amine moiety, azide moiety, alkyne moiety, dibenzocyclooctyne (DBCO) moiety, epoxy moiety, or isocyanate moiety. In embodiments, the bioconjugate reactive moiety is an azido moiety.

In embodiments, each particle includes multiple copies of one or more oligonucleotide moieties. In embodiments, the one or more oligonucleotide moieties include at least two different primers attached to the polymer (e.g., a forward and a reverse primer), each of which may be present in multiple copies. In embodiments, about or at most at most about 50%, 40%, 30%, 25%, 20%, 15%, 10%, 5%, or less of the monomers in the polymer of each particle are attached to a copy of the oligonucleotide moiety. In embodiments, about 1-25%, about 2-20%, about 3-15%, about 4-14%, or about 5-12% of the monomers in the polymer of each particle are attached to a copy of the oligonucleotide moiety, or a number or a range between any two of these values. In embodiments, about 5-10% of the monomers in the polymer of each particle are attached to a copy of the oligonucleotide moiety. In embodiments, two different oligonucleotide moieties are attached to the particle (e.g., a forward and a reverse primer), which facilitates generating multiple amplification products from the first extension product or a complement thereof.

In embodiments, the first primer is immobilized on the substrate via a first linker and the second primer is immobilized to the substrate via a second linker. The linkers may also include spacer nucleotides. Including spacer nucleotides in the linker puts the polynucleotide in an environment having a greater resemblance to free solution. This can be beneficial, for example, in enzyme-mediated reactions such as sequencing-by-synthesis. It is believed that such reactions suffer less steric hindrance issues that can occur when the polynucleotide is directly attached to the solid support or is attached through a very short linker (e.g., a linker including about 1 to 3 carbon atoms). Spacer nucleotides form part of the polynucleotide but do not participate in any reaction carried out on or with the polynucleotide (e.g., a hybridization or amplification reaction). In embodiments, the spacer nucleotides include 1 to 20 nucleotides. In embodiments, the linker includes 10 spacer nucleotides. In embodiments, the linker includes 12 spacer nucleotides. In embodiments, the linker includes 15 spacer nucleotides. It is preferred to use polyT spacers, although other nucleotides and combinations thereof can be used. In embodiments, the linker includes 10, 11, 12, 13, 14, or 15 T spacer nucleotides. In embodiments, the linker includes 12 T spacer nucleotides. Spacer nucleotides are typically included at the 5′ ends of polynucleotides which are attached to a suitable support. Attachment can be achieved via a phosphorothioate present at the 5′ end of the polynucleotide, an azide moiety, a dibenzocyclooctyne (DBCO) moiety, or any other bioconjugate reactive moiety. The linker may be a carbon-containing chain such as those of formula —(CH₂)n- wherein “n” is from 1 to about 1000. However, a variety of other linkers may be used so long as the linkers are stable under conditions used in DNA sequencing. In embodiments, the linker includes polyethylene glycol (PEG) having a general formula of —(CH₂—CH₂—O)m-, wherein m is from about 1 to 500, or from about 1 to 100, from about 1 to 50 or from about 1 to about 12. In embodiments, the linker, or the immobilized oligonucleotides (e.g., primers) include one or more cleavable site(s). In embodiments, a cleavable site is a location which allows controlled cleavage of the immobilized polynucleotide strand (e.g., the linker, the primer, or the polynucleotide) by chemical, enzymatic or photochemical means. In embodiments, the cleavable site includes one or more deoxyuracil nucleobases (dUs). Any suitable enzymatic, chemical, or photochemical cleavage reaction may be used to cleave the cleavable site. The cleavage reaction may result in removal of a part or the whole of the strand being cleaved. Suitable cleavage means include, for example, restriction enzyme digestion, in which case the cleavable site is an appropriate restriction site for the enzyme which directs cleavage of one or both strands of a duplex template; RNase digestion or chemical cleavage of a bond between a deoxyribonucleotide and a ribonucleotide, in which case the cleavable site may include one or more ribonucleotides; chemical reduction of a disulfide linkage with a reducing agent (e.g., THPP or TCEP), in which case the cleavable site should include an appropriate disulfide linkage; chemical cleavage of a diol linkage with periodate, in which case the cleavable site should include a diol linkage; generation of an abasic site and subsequent hydrolysis, etc. In embodiments, the cleavable site is included in the surface immobilized primer (e.g., within the polynucleotide sequence of the primer). In embodiments, the linker, the primer, or the first or second polynucleotide includes a diol linkage which permits cleavage by treatment with periodate (e.g., sodium periodate). It will be appreciated that more than one diol can be included at the cleavable site. One or more diol units may be incorporated into a polynucleotide using standard methods for automated chemical DNA synthesis. Polynucleotide primers including one or more diol linkers can be conveniently prepared by chemical synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, each of the plurality of immobilized oligonucleotides (e.g., immobilized primers) is about 5 to about 25 nucleotides in length. In embodiments, each of the plurality of immobilized oligonucleotides (e.g., immobilized primers) is about 20 to about 50 nucleotides in length. In embodiments, each of the plurality of immobilized oligonucleotides (e.g., immobilized primers) is about 10 to about 40 nucleotides in length. In embodiments, each of the plurality of immobilized oligonucleotides (e.g., immobilized primers) is about 5 to about 100 nucleotides in length. In embodiments, each of the plurality of immobilized oligonucleotides (e.g., immobilized primers) is about 20 to 200 nucleotides in length. In embodiments, each of the plurality of immobilized oligonucleotides (e.g., immobilized primers) about or at least about 5, 6, 7, 8, 9, 10, 12, 15, 18, 20, 25, 30, 35, 40, 50 or more nucleotides in length. In other embodiments, each of the plurality of immobilized oligonucleotides (e.g., immobilized primers) is about 100 to about 200 nucleotides in length. In other embodiments, each of the plurality of immobilized oligonucleotides (e.g., immobilized primers) is about 125 to about 175 nucleotides in length. In other embodiments, each of the plurality of immobilized oligonucleotides (e.g., immobilized primers) is about 150 nucleotides in length. In some embodiments, the oligonucleotide moiety is about 5 to about 50 nucleotides in length. In some embodiments, the oligonucleotide moiety is about 5 to about 40 nucleotides in length. In some embodiments, the oligonucleotide moiety is about 10 to about 45 nucleotides in length. In some embodiments, the oligonucleotide moiety is about 15 to about 40 nucleotides in length. In some embodiments, the oligonucleotide moiety is about 20 to about 35 nucleotides in length. In some embodiments, the oligonucleotide moiety is about 20 to about 30 nucleotides in length. In some embodiments, the oligonucleotide moiety is about 25 to about 30 nucleotides in length. In embodiments, the oligonucleotide moiety is about 25 to about 35 nucleotides in length. In embodiments, the oligonucleotide moiety is about 30 to about 50 nucleotides in length. In embodiments, the oligonucleotide moiety is about 30 to about 75 nucleotides in length. In embodiments, the oligonucleotide moiety is about 50 to about 150 nucleotides in length. In embodiments, the oligonucleotide moiety is about 75 to about 200 nucleotides in length. In embodiments, the oligonucleotide moiety is a capture oligonucleotide, wherein the oligonucleotide is capable of hybridizing to a common sequence in a library of nucleic acid molecules.

In embodiments, one or more immobilized oligonucleotides include blocking groups at their 3′ ends that prevent polymerase extension. A blocking moiety prevents formation of a covalent bond between the 3′ hydroxyl moiety of the nucleotide and the 5′ phosphate of another nucleotide. In embodiments, the 3′ modification is a 3′-phosphate modification, including a 3′ phosphate moiety, which is removed by a PNK enzyme or a phosphatase enzyme. Alternatively, abasic site cleavage with certain endonucleases (e.g., Endo IV) results in a 3′-OH at the cleavable site from the 3′-diesterase activity.

In embodiments, each site (e.g., well of a multiwell container, amplification site, or particle) includes oligonucleotide moieties substantially identical to all the other sites. In embodiments, each of the sites include at least two species (i.e., two populations) of oligonucleotide moieties that are substantially identical to all the sites. In embodiments, each site includes substantially the same oligonucleotide moieties (e.g., a first population of oligonucleotide moieties and a second population of oligonucleotide moieties). In embodiments, each site includes least two species of substantially the same oligonucleotide moieties (i.e., the same sequences). In embodiments, the oligonucleotide is capable of hybridizing to a common sequence (e.g., a sequence described in U.S. Patent Publication 2016/0256846, which is incorporated herein by reference, for example SEQ ID NO: 3, SEQ ID NO: 4, SEQ ID NO: 5, or SEQ ID NO: 11 of U.S. Patent Publication 2016/0256846). In embodiments, the support includes about 10²-10¹⁵ immobilized first oligonucleotide moieties per mm². In embodiments, the support includes about 10²-10¹⁵ immobilized second oligonucleotide moieties per mm². In embodiments, the support includes about 10⁸-10¹² immobilized first oligonucleotide moieties per mm². In embodiments, the support includes about 10⁸-10¹² immobilized second oligonucleotide moieties per mm².

In embodiments, each site includes a plurality of P7 or P5 nucleic acid sequences or complementary sequences thereof (i.e., P5′ or P7′). The P5 and P7 adapter sequences are described in U.S. Patent Publication No. 2011/0059865 A1, which is incorporated herein by reference in its entirety. The terms P5 and P7 may be used when referring to amplification primers, e.g., universal primers. The terms P5′ (P5 prime) and P7′ (P7 prime) refer to the complement of P5 and P7, respectively. In embodiments, each particle includes a first plurality of a platform primer sequence and a second plurality of a differing platform primer sequence. In embodiments, the platform primer sequence is used during amplification reactions (e.g., solid phase amplification). In embodiments, each particle includes oligonucleotide moieties capable of annealing to an adapter of a library nucleic acid molecule. The term “library” merely refers to a collection or plurality of template nucleic acid molecules which share common sequences at their 5′ ends (e.g., the first end) and common sequences at their 3′ ends (e.g., the second end). The term “adapter” as used herein refers to any linear oligonucleotide that can be ligated to a nucleic acid molecule, thereby generating nucleic acid products that can be sequenced on a sequencing platform (e.g., an Illumina or Singular Genomics' G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In embodiments, each of the particles include at least two populations of substantially the same oligonucleotide moieties. In embodiments, the solid support includes a plurality of immobilized oligonucleotides. In embodiments, the solid support includes a plurality of oligonucleotides immobilized to a polymer. In embodiments, the solid support includes a plurality of particles. In embodiments, the solid support includes a first plurality of immobilized oligonucleotides. In embodiments, the solid support includes a first and a second plurality of immobilized oligonucleotides, wherein the immobilized oligonucleotides of each plurality are different (e.g., S1 or S2).

In embodiments, the plurality of oligonucleotides is present at a density of about 100 oligonucleotides per μm² to about 1,000,000 oligonucleotides per μm². In embodiments, the plurality of oligonucleotides is present at a density of about 100 oligonucleotides per μm² to about 1,000 oligonucleotides per μm². In embodiments, the plurality of oligonucleotides is present at a density of about 100 oligonucleotides per μm² to about 10,000 oligonucleotides per μm². In embodiments, the plurality of oligonucleotides is present at a density of about 100 oligonucleotides per μm² to about 100,000 oligonucleotides per μm². In embodiments, the plurality of oligonucleotides is present at a density of about 100 oligonucleotides per μm² to about 500,000 oligonucleotides per μm². In embodiments, the plurality of oligonucleotides is present at a density of about 100, 1,000, 10,000, 50,000, 100,000, 250,000, 500,000, 750,000, or 1,000,000 oligonucleotides per μm².

In embodiments, one or more immobilized oligonucleotides include blocking groups at their 3′ ends that prevent polymerase extension. A blocking moiety prevents formation of a covalent bond between the 3′ hydroxyl moiety of the nucleotide and the 5′ phosphate of another nucleotide. In embodiments, the 3′ modification is a 3′-phosphate modification, including a 3′ phosphate moiety, which is removed by a PNK enzyme or a phosphatase enzyme. Alternatively, abasic site cleavage with certain endonucleases (e.g., Endo IV) results in a 3′-OH at the cleavable site from the 3′-diesterase activity. As described in US2010/0167353, a number of blocking groups are known in the art that can be placed at or near the 3′ end of the oligonucleotide (e.g., a primer) to prevent extension. A primer or other oligonucleotide may be modified at the 3′-terminal nucleotide to prevent or inhibit initiation of DNA synthesis by, for example, the addition of a 3′ deoxyribonucleotide residue (e.g., cordycepin), a 2′,3′-dideoxyribonucleotide residue, non-nucleotide linkages or alkane-diol modifications (as described in U.S. Pat. No. 5,554,516). Alkane diol modifications which can be used to inhibit or block primer extension have also been described by Wilk et al., (1990 Nucleic Acids Res. 18 (8):2065), and by Arnold et al. (U.S. Pat. No. 6,031,091). Additional examples of suitable blocking groups include 3′ hydroxyl substitutions (e.g., 3′-phosphate, 3′-triphosphate or 3′-phosphate diesters with alcohols such as 3-hydroxypropyl), 2′3′-cyclic phosphate, 2′ hydroxyl substitutions of a terminal RNA base (e.g., phosphate or sterically bulky groups such as triisopropyl silyl (TIPS) or tert-butyl dimethyl silyl (TBDMS)). 2′-alkyl silyl groups such as TIPS and TBDMS substituted at the 3′-end of an oligonucleotide are described in US 2007/0218490, which is incorporated herein by reference. Bulky substituents and/or reversible terminators can also be incorporated on the base of the 3′-terminal residue of the oligonucleotide to block primer extension. In certain embodiments, the oligonucleotide may include a cleavage domain that is located upstream (e.g., 5′ to) of the blocking group used to inhibit primer extension. As examples, the cleavage domain may be an RNase H cleavage domain, or the cleavage domain may be an RNase H2 cleavage domain including a single RNA residue, or the oligonucleotide may include replacement of the RNA base with one or more alternative nucleosides. Additional illustrative cleavage domains are described in US2010/0167353.

In some embodiments, the oligonucleotide moiety is capable of hybridizing to a complementary sequence of a template nucleic acid. In embodiments, the oligonucleotide moiety includes DNA. In embodiments, the oligonucleotide moiety includes RNA. In embodiments, the oligonucleotide moiety is DNA. In embodiments, the oligonucleotide moiety is RNA. In embodiments, the oligonucleotide moiety includes a single-stranded DNA. In embodiments, the oligonucleotide moiety includes a single-stranded RNA. In embodiments, the oligonucleotide moiety is a single-stranded DNA. In embodiments, the oligonucleotide moiety is a single-stranded RNA. In embodiments, the oligonucleotide moiety is a nucleic acid sequence complementary to a target polynucleotide (e.g., complementary to a common adapter sequence of the target polynucleotide).

In embodiments, the immobilized primers are designed to have a particular melting temperature (Tm). The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). In embodiments, the Tm of the immobilized primers have a Tm of about 55° C. to about 70° C. In embodiments, the Tm of the immobilized primers have a Tm of about 60° C. to about 70° C. In embodiments, the Tm of the immobilized primers have a Tm of about 60° C. to about 65° C.

In embodiments, the immobilized primers includes one or more phosphorothioate nucleotides. In embodiments, the immobilized primers includes a plurality of phosphorothioate nucleotides. In embodiments, about or at least about 20%, 30%, 40%, 50%, 60%, 70%, 80%, 90%, or about 100% of the nucleotides in the immobilized primers are phosphorothioate nucleotides. In embodiments, most of the nucleotides in the immobilized primers are phosphorothioate nucleotides. In embodiments, all of the nucleotides in the immobilized primers are phosphorothioate nucleotides. In embodiments, none of the nucleotides in the immobilized primers are phosphorothioate nucleotides. In embodiments, the 5′ end of the immobilized primers includes one or more phosphorothioate nucleotides. In embodiments, the 5′ end of the immobilized primers includes between one and five phosphorothioate nucleotides.

In embodiments, the first and second primer polynucleotides are each attached to the solid support (i.e., immobilized on the surface of a solid support). The polynucleotide molecules can be fixed to surface by a variety of techniques, including covalent attachment and non-covalent attachment. In embodiments, the polynucleotides are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments, the solid support includes a plurality of immobilized primers. In embodiments, the solid support includes a plurality of non-extended immobilized primers.

In embodiments of the methods and compositions provided herein, the clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4., 4.5, 4.6, 4.7, 4.8, 4.9, 5.0 μm or a number or a range between any two of these values. In embodiments, the mean or median separation is about 0.1-10 microns. In embodiments, the mean or median separation is about 0.25-5 microns. In embodiments, the mean or median separation is about 0.5-2 microns. In embodiments, the mean or median separation is about or at least about 0.1 μm. In embodiments, the mean or median separation is about or at least about 0.25 μm. In embodiments, the mean or median separation is about or at least about 0.5 μm. In embodiments, the mean or median separation is about or at least about 1.0 μm. In embodiments, the mean or median separation is about or at least about 2.0 μm. In embodiments, the mean or median separation is about or at least about 5.0 μm. In embodiments, the mean or median separation is about or at least about 10 μm. The mean or median separation may be measured center-to-center (i.e., the center of one cluster to the center of a second cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm.

In embodiments of the methods provided herein, the amplicon clusters have a mean or median diameter of about 100-2,000 nm, or about 200-1,000 nm. In embodiments, the mean or median diameter is about 100-3,000 nanometers, about 500-2,500 nanometers, about 1,000-2,000 nanometers, or a number or a range between any two of these values. In embodiments, the mean or median diameter is about or at most about 100, 200, 300, 400, 500, 600, 700, 800, 900, 1,000, 1,100, 1,200, 1,300, 1,400, 1,500, 1,600, 1,700, 1,800, 1,900, 2000 nanometers or a number or a range between any two of these values. In embodiments, the mean or median diameter is about 100-3,000 nanometers. In embodiments, the mean or median diameter is about 100-2,000 nanometers. In embodiments, the mean or median diameter is about 500-2,500 nanometers. In embodiments, the mean or median diameter is about 200-1,000 nanometers. In embodiments, the mean or median diameter is about 1,000-2,000 nanometers. In embodiments, the mean or median diameter is about or at most about 100 nanometers. In embodiments, the mean or median diameter is about or at most about 200 nanometers. In embodiments, the mean or median diameter is about or at most about 500 nanometers. In embodiments, the mean or median diameter is about or at most about 700 nanometers. In embodiments, the mean or median diameter is about or at most about 1,000 nanometers. In embodiments, the mean or median diameter is about or at most about 2,000 nanometers. In embodiments, the mean or median diameter is about or at most about 2,500 nanometers. In embodiments, the mean or median diameter is about or at most about 3,000 nanometers.

In embodiments, the template polynucleotide includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the template polynucleotide includes double-stranded DNA. In embodiments, the method of forming the template polynucleotide includes ligating a hairpin adapter to an end of a linear polynucleotide. In embodiments, the method of forming the template polynucleotide includes ligating hairpin adapters to both ends of the linear polynucleotide. In embodiments, the method of forming the template polynucleotide includes ligating a Y-shaped adapter to an end of a linear polynucleotide. In embodiments, the method of forming the template polynucleotide includes ligating a Y-shaped adapter to both ends of a linear polynucleotide.

In some embodiments, a double stranded nucleic acid (i.e., a duplex) includes two complementary nucleic acid strands. In embodiments, a double stranded nucleic acid includes a first strand and a second strand which are complementary or substantially complementary to each other. A first strand of a double stranded nucleic acid is sometimes referred to herein as a forward strand and a second strand of the double stranded nucleic acid is sometime referred to herein as a reverse strand. In some embodiments, a double stranded nucleic acid includes two opposing ends. Accordingly, a double stranded nucleic acid often includes a first end and a second end. An end of a double stranded nucleic acid may include a 5′-overhang, a 3′-overhang or a blunt end. In some embodiments, one or both ends of a double stranded nucleic acid are blunt ends. In certain embodiments, one or both ends of a double stranded nucleic acid are manipulated to include a 5′-overhang, a 3′-overhang or a blunt end using a suitable method. In some embodiments, one or both ends of a double stranded nucleic acid are manipulated during library preparation such that one or both ends of the double stranded nucleic acid are configured for ligation to an adapter using a suitable method. For example, one or both ends of a double stranded nucleic acid may be digested by a restriction enzyme, polished, end-repaired, filled in, phosphorylated (e.g., by adding a 5′-phosphate), dT-tailed, dA-tailed, the like or a combination thereof.

In embodiments, the double stranded nucleic acid, alternatively referred to as a library insert or target polynucleotide, is at least 50, 100, 150, 200, 250, or 300 nucleotides in length. In embodiments, the double stranded nucleic acid, alternatively referred to as a library insert, is at least 150, 200, 250, 300, 350, or 400 nucleotides in length. In embodiments, the double stranded nucleic acid, alternatively referred to as a library insert, is at least 450, 500, 650, 700, 750, or 800 nucleotides in length. In embodiments, the double stranded nucleic acid, alternatively referred to as a library insert, is at least 850, 900, 950, 1000, 1050, or 1100 nucleotides in length.

In embodiments, the double stranded nucleic acid, alternatively referred to as a library insert, is about 50, 100, 150, 200, 250, or 300 nucleotides in length. In embodiments, the double stranded nucleic acid, alternatively referred to as a library insert, is about 150, 200, 250, 300, 350, or 400 nucleotides in length. In embodiments, the double stranded nucleic acid, alternatively referred to as a library insert, is about 450, 500, 650, 700, 750, or 800 nucleotides in length. In embodiments, the double stranded nucleic acid, alternatively referred to as a library insert, is about 850, 900, 950, 1000, 1050, or 1100 nucleotides in length. In embodiments, the double stranded nucleic acid, alternatively referred to as a library insert, is about 500-1500 nucleotides in length. In embodiments, the double stranded nucleic acid, alternatively referred to as a library insert, is about 750-1500 nucleotides in length. In embodiments, the double stranded nucleic acid, alternatively referred to as a library insert, is about 1-2 kilobases (kb) in length. In embodiments, the double stranded nucleic acid, alternatively referred to as a library insert, is about 300, 400, 600, or 800 nucleotides in length. In embodiments, the double stranded nucleic acid, alternatively referred to as a library insert, is about 250 to 600 nucleotides in length.

In embodiments, the double stranded nucleic acid is about 100, 125, 150, 175, or 200 nucleotides in length. In embodiments, the double stranded nucleic acid is about 200, 225, 250, 275, or 300 nucleotides in length. In embodiments, the double stranded nucleic acid is less than 150 nucleotides in length. In embodiments, the double stranded nucleic acid is less than 100 nucleotides in length. In embodiments, the double stranded nucleic acid is less than 75 nucleotides in length. In embodiments, the double stranded nucleic acid is about 150 nucleotides in length. In embodiments, the double stranded nucleic acid is about 100 nucleotides in length. In embodiments, the double stranded nucleic acid is about 75 nucleotides in length. In embodiments, the method provides sequencing both strands of a double stranded nucleic acid such that there is overlap in the sequencing reads of the first and second strand. For example, if the double stranded nucleic acid is short (e.g., 150-200 nucleotides) it is possible to sequence the first strand and a complementary region of the second strand (e.g., in the same read).

In embodiments, the double stranded nucleic acid is greater than 150 nucleotides in length. In embodiments, the double stranded nucleic acid is greater than 200 nucleotides in length. In embodiments, the double stranded nucleic acid is greater than 250 nucleotides in length. In embodiments, the double stranded nucleic acid is greater than 300 nucleotides in length. In embodiments, the double stranded nucleic acid is greater than 500 nucleotides in length. In embodiments, the double stranded nucleic acid is greater than 700 nucleotides in length. In embodiments, the double stranded nucleic acid is greater than 900 nucleotides in length. In embodiments, the double stranded nucleic acid is greater than 1,000 nucleotides in length (i.e., greater than 1 kb). In embodiments, the method provides sequencing both strands of a double stranded nucleic acid such that there is no overlap in the sequencing reads of the first and second strand, rather a portion of the first strand and portion of the second strand.

In embodiments, the template polynucleotide is about 100 to 1,000 nucleotides in length. In embodiments, the template polynucleotide is about 350 nucleotides in length. In embodiments, the template polynucleotide is about 10, 20, 50, 100, 150, 200, 300, or 500 nucleotides in length. The template polynucleotide molecules can vary length, such as about 100-300 nucleotides long, about 300-500 nucleotides long, or about 500-1,000 nucleotides long. In embodiments, the template polynucleotide molecular is about 100-1,000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the template polynucleotide molecule is about 150 nucleotides. In embodiments, the template polynucleotide is about 100-1,000 nucleotides long. In embodiments, the template polynucleotide is about 100-300 nucleotides long. In embodiments, the template polynucleotide is about 300-500 nucleotides long. In embodiments, the template polynucleotide is about 500-1,000 nucleotides long. In embodiments, the template polynucleotide molecule is about 100 nucleotides. In embodiments, the template polynucleotide molecule is about 300 nucleotides. In embodiments, the template polynucleotide molecule is about 500 nucleotides. In embodiments, the template polynucleotide molecule is about 1,000 nucleotides.

In embodiments the template polynucleotide (e.g., genomic template DNA) is first treated to form single-stranded linear fragments (e.g., ranging in length from about 50 to about 600 nucleotides). Treatment typically entails fragmentation, such as by chemical fragmentation, enzymatic fragmentation, or mechanical fragmentation, followed by denaturation to produce single-stranded DNA fragments. In embodiments, the template polynucleotide includes an adapter. The adapter may have other functional elements including tagging sequences (i.e., a barcode), attachment sequences, palindromic sequences, restriction sites, sequencing primer binding sites, functionalization sequences, and the like. Barcodes can be of any of a variety of lengths. In embodiments, the primer includes a barcode that is 10-50, 20-30, or 4-12 nucleotides in length. In embodiments, the adapter includes a primer binding sequence that is complementary to at least a portion of a primer (e.g., a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length.

In some embodiments, the methods described herein includes ligating one or more adapters to a double stranded nucleic acid. In some embodiments, the methods described herein includes ligating one or more adapters to a plurality of double stranded nucleic acids. In some embodiments, the methods described herein includes ligating a first adapter to a first end of a double stranded nucleic acid and ligating a second adapter to a second end of a double stranded nucleic acid. In some embodiments, the first adapter and the second adapter are different (e.g., non-identical adapters). For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

In embodiments, the first adapter is a Y-adapter. In embodiments, the Y-adapter includes (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand. In embodiments, the 5′-arm of the first strand or the 3′-arm of the second strand of the Y-adapter includes a melting temperature (Tm) in a range of 60-85° C. In embodiments, the blocking primer anneals to the 5′-portion of the second strand of the Y-adapter.

In embodiments, the first adapter is a hairpin adapter. In some embodiments, the hairpin adapter includes a 5′-end, a 5′-portion, the loop, a 3′-portion and a 3′-end, and the 5′-portion of the hairpin adapter is substantially complementary to the 3′-portion of the hairpin adapter. In embodiments, the blocking primer anneals to a sequence within a loop of the first adapter.

In some embodiments, the first adapter is a Y-adapter, and annealing a blocking primer includes: (i) hybridizing a blocking primer to a single-stranded portion of the Y-adapter, and (ii) extending the blocking primer with a strand-displacing polymerase that terminates extension within a loop of the hairpin adapter at a terminating nucleotide.

In embodiments, the terminating nucleotide includes a removable group that blocks progression of the strand-displacing polymerase, and further wherein the terminating nucleotide is treated to release the removable group prior to sequencing. Any of a variety of suitable modifications capable of terminating strand extensions may be used. In general, the terminating nucleotide is the nucleotide position that is modified to inhibit strand extension. The terminating nucleotide may or may not be a nucleotide analog. Thus, a terminating nucleotide is not necessarily chemically modified. For example, a terminating nucleotide may be a naturally occurring nucleotide, but is bound by another factor that inhibits strand extension (such as a sequence-specific binding protein). Any of a variety of suitable chemical modifications and blocking groups may be used. In embodiments, the terminating nucleotide is a nucleotide analog. Non-limiting examples include C3′-modifications, C2′-modifications, and phosphorodithioates.

In embodiments, the removable group is a polymer or a protein joined to the terminating nucleotide by a cleavable linker. In embodiments, the removable group is a polymer, such as a dendrimer. Non-limiting examples of polymers include PEG, polyethyleneimine, and poly(amidoamide). In embodiments, the protein is a bovine serum albumin (BSA).

In embodiments, the removable group is a protein that is non-covalently complexed to the terminating nucleotide, and further wherein releasing the protein includes a change in reaction conditions to disrupt the complex. The nature of the change in reaction conditions will depend on the nature of the protein complexed to the terminating nucleotide. In embodiments, the change in reaction conditions includes a change in temperature. In embodiments, the change in reaction conditions includes a change in buffer conditions, such as an increase in salt concentration. In embodiments, the change in reactions conditions includes the addition of another agent that competes with, inhibits, or degrades the protein.

In embodiments, the protein is a first member of a binding pair complexed with a second member of the binding pair that is linked to the terminating nucleotide. In embodiments, the protein is a single-stranded binding protein that recognizes a sequence within the loop of the hairpin adapter. In embodiments, the binding pair is a binding pair as described with respect to other aspects disclosed herein, including with respect to methods of sequencing described herein.

In embodiments, the terminating nucleotide is a first nucleotide analog that base pairs with a second nucleotide analog, and the second nucleotide analog is not present in the primer extension reaction, such that primer extension terminates.

In some embodiments, each strand of a Y-adapter, each of the non-complementary arms of a Y-adapter, or a duplex portion of a Y-adapter has a length independently selected from at least 5, at least 10, at least 15, at least 25, and at least 40 nucleotides. In some embodiments, each strand of a Y-adapter, each of the non-complementary arms of a Y-adapter, or a duplex portion of a Y-adapter has a length in a range independently selected from 15 to 500 nucleotides, 15-250 nucleotides, 15 to 200 nucleotides, 15 to 150 nucleotides, 20 to 100 nucleotides, 20 to 50 nucleotides and 10-50 nucleotides. In embodiments, one or both non-complementary arms of the Y-adapter is about or at least about 10, 15, 20, 25, 30, 35, 40, 45, 50, or more nucleotides in length. In embodiments, one or both non-complementary arms of the Y-adapter is about or at least about 20 nucleotides in length. In embodiments, one or both non-complementary arms of the Y-adapter is about or at least about 30 nucleotides in length. In embodiments, one or both non-complementary arms of the Y-adapter is about or at least about 40 nucleotides in length. In embodiments, the duplex portion of a Y-adapter is about or at least about 5, 10, 15, 20, 25, 30, or more nucleotides in length. In embodiments, the duplex portion of a Y-adapter is about 5-50, 5-25, or 10-15 nucleotides in length. In embodiments, the duplex portion of a Y-adapter is about or at least about 10 nucleotides in length. In embodiments, the duplex portion of a Y-adapter is about or at least about 15 nucleotides in length. In embodiments, the duplex portion of a Y-adapter is about or at least about 12 nucleotides in length. In embodiments, the duplex portion of a Y-adapter is about or at least about 20 nucleotides in length.

In some embodiments, a Y-adapter includes a first end including a duplex region including double stranded nucleic acid, and a second end including a forked region, where the first end is configured for ligation to an end of double stranded nucleic acid (e.g., a nucleic acid fragment, e.g., a library insert). In embodiments, a duplex end of a Y-adapter includes a 5′-overhang or a 3′-overhang that is complementary to a 3′-overhang or a 5′-overhang of an end of a double stranded nucleic acid. In some embodiments, a duplex end of a Y-adapter includes a blunt end that can be ligated to a blunt end of a double stranded nucleic acid. In certain embodiment, a duplex end of a Y-adapter includes a 5′-end that is phosphorylated.

In some embodiments, the first and/or second adapter (e.g., one or both strands of a Y-adapter) include one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In some embodiments, a non-complementary portion (e.g., 5′-arm and/or 3′-arm) of a Y-adapter includes one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In certain embodiments, a non-complementary portion of a Y-adapter includes a primer binding site. In certain embodiments, a non-complementary portion of a Y-adapter includes a binding site for a capture nucleic acid. In certain embodiments, a non-complementary portion of a Y-adapter includes a primer binding site and a UMI. In certain embodiments, a non-complementary portion of a Y-adapter includes a binding motif. In embodiments, the first and/or second adapter (e.g., one or both strands of a Y-adapter) does not include a UMI or sample barcode.

In certain embodiments, a complementary strand (e.g., a 3′-portion or 5′-portion) of a Y-adapter includes a primer binding site. In certain embodiments, a complementary strand (e.g., a 3′-portion or 5′-portion) of a Y-adapter includes a binding site for a capture nucleic acid. In certain embodiments, a complementary strand (e.g., a 3′-portion or 5′-portion) of a Y-adapter includes a primer binding site and a UMI. In certain embodiments, a complementary strand (e.g., a 3′-portion or 5′-portion) of a Y-adapter includes a binding motif.

In some embodiments, each of the non-complementary portions (i.e., arms) of a Y-adapter independently have a predicted, calculated, mean, average or absolute melting temperature (Tm) that is greater than 50° C., greater than 55° C., greater than 60° C., greater than 65° C., greater than 70° C. or greater than 75° C. In some embodiments, each of the non-complementary portions of a Y-adapter independently have a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm is about or at least about 70° C. In embodiments, the Tm is about or at least about 75° C. In embodiments, the Tm is about or at least about 80° C. In embodiments, the Tm is a calculated Tm. Tm's are routinely calculated by those skilled in the art, such as by commercial providers of custom oligonucleotides. In embodiments, the Tm for a given sequence is determined based on that sequence as an independent oligo. In embodiments, Tm is calculated using web-based algorithms, such as Primer3 and Primer3Plus (www.bioinformatics.nl/cgi-bin/primer3plus/primer3plus.cgi) using default parameters. The Tm of a non-complementary portion of a Y-adapter can be changed (e.g., increased) to a desired Tm using a suitable method, for example by changing (e.g., increasing) GC content, changing (e.g., increasing) length and/or by the inclusion of modified nucleotides, nucleotide analogs and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, each of the non-complementary portion of a Y-adapter independently include one or more modified nucleotides, nucleotide analogs and/or modified nucleotides bonds.

In some embodiments, each of the non-complementary portions of a Y-adapter independently include a GC content of greater than 40%, greater than 50%, greater than 55%, greater than 60% greater than 65% or greater than 70%. In certain embodiments, each of the non-complementary portions of a Y-adapter independently include a GC content in a range of 40-100%, 50-100%, 60-100% or 70-100%. In embodiments, one or both non-complementary portions of a Y-adapter have a GC content of about or more than about 40%. In embodiments, one or both non-complementary portions of a Y-adapter have a GC content of about or more than about 50%. In embodiments, one or both non-complementary portions of a Y-adapter have a GC content of about or more than about 60%. Non-base modifiers can also be incorporated into a non-complementary portion of a Y-adapter to increase Tm, non-limiting examples of which include a minor grove binder (MGB), spermine, G-clamp, a Uaq anthraquinone cap, the like or combinations thereof.

In certain embodiments, a duplex region of a Y-adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of a duplex region of the Y-adapter is about or more than about 30° C. In embodiments, the Tm of a duplex region of the Y-adapter is about or more than about 35° C. In embodiments, the Tm of a duplex region of the Y-adapter is about or more than about 40° C. In embodiments, the Tm of a duplex region of the Y-adapter is about or more than about 45° C. In embodiments, the Tm of a duplex region of the Y-adapter is about or more than about 50° C.

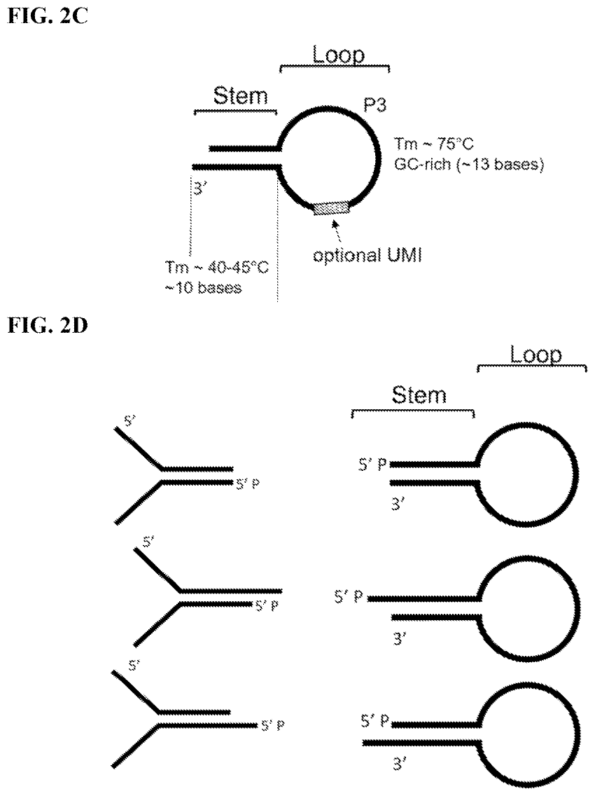

In some embodiments, an adapter is hairpin adapter. In some embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75 to 500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a hairpin adapter includes a structure described herein (e.g., FIGS. 2B-2D). In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15 to 250 nucleotides, 15 to 200 nucleotides, 15 to 150 nucleotides, 20 to 100 nucleotides or 20 to 50 nucleotides.

In embodiments, ligating includes ligating both the 3′ end and the 5′ end of the duplex region of the second adapter to the double stranded nucleic acid. In embodiments, ligating includes ligating either the 3′ end or the 5′ end of the duplex region of the second adapter to the double stranded nucleic acid. In embodiments, ligating includes ligating the 5′ end of the duplex region of the second adapter to the double stranded nucleic acid and not the 3′ end of the duplex region.

In some embodiments, a loop of a hairpin adapter includes one or more of a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, a loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogs and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogs and/or modified nucleotides bonds.

In some embodiments, a loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loops has a GC content of about or more than about 40%. In embodiments, the loops has a GC content of about or more than about 50%. In embodiments, the loops has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15 to 250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In some embodiments, the first adapter is a hairpin adapter, and annealing a blocking primer includes: (i) hybridizing a blocking primer within a loop of the first hairpin adapter, and (ii) extending the blocking primer with a strand-displacing polymerase that terminates extension within a loop of the second hairpin adapter at a terminating nucleotide.

In embodiments, the Y-adaptor portion of a Y-adaptor-ligated double-stranded nucleic acid is formed from cleavage in the loop of a hairpin adapter (e.g., one or more adapters as described in U.S. Pat. No. 8,883,990, which is incorporated herein by reference for all purposes). For example, in embodiments disclosed herein relating to ligation to a Y-adapter, ligation may instead be to a hairpin adapter, followed by cleavage within the loop of the hairpin adapter to release two unpaired ends. In embodiments, a hairpin adapter includes one or more uracil nucleotide(s) in the loop, and cleavage in the loop may be accomplished by the combined activities of Uracil DNA glycosylase (UDG) and the DNA glycosylase-lyase Endonuclease VIII, or suitable cleavage conditions known in the art. UDG cleaves the glycosidic bond between the deoxyribose of the DNA sugar-phosphate backbone and the uracil base, and Endonuclease VIII cleaves the AP site, effectively cleaving the loop. In embodiments, the hairpin adapter includes a recognition sequence for a compatible restriction enzyme. In embodiments, the hairpin adapter includes one or more ribonucleotides and cleavage in the loop is accomplished by RNase H. In embodiments, the loop of the hairpin adapter includes a cleavable linkage (e.g., a cleavable site) that is positioned between two non-complementary regions of the loop. In embodiments, the non-complementary region that is 5′ of the cleavable linkage includes a primer binding site that is in the range of 8 to 100 nucleotides in length. In embodiments, the first adapter is a hairpin adapter, wherein the hairpin adapter includes a cleavable site in the loop. In embodiments, the first adapter is a first hairpin adapter and the second adapter is a hairpin adapter, wherein only the first hairpin adapter includes a cleavable site in the loop.

In some embodiments, a method includes sequencing a template described herein. In some embodiments, the sequencing includes contacting the template with a suitable polymerase. In certain embodiments, the polymerase is in an aqueous phase. In certain embodiments, the polymerase is soluble in an aqueous solution. In some embodiments, the polymerase is not attached to a substrate. In some embodiments, the polymerase is attached to a substrate. In embodiments, the polymerase is a mutant polymerase capable of incorporating modified nucleotides.

In embodiments, the terminating nucleotide includes a removable group that blocks progression of the strand-displacing polymerase, and further wherein the terminating nucleotide is treated to release the removable group prior to sequencing.

In embodiments, the terminating nucleotide is an RNA nucleotide.

In embodiments, annealing a blocking primer includes (i) forming a complex including a portion of the double-stranded nucleic acid, a blocking primer, and a homologous recombination complex including a recombinase, (ii) releasing the recombinase, and (iii) extending the blocking primer with a strand-displacing polymerase.

In embodiments, (i) annealing a blocking primer includes forming a complex including a portion of the double-stranded nucleic acid, a probe oligonucleotide, and a homologous recombination complex including a recombinase, and (ii) annealing a probe oligonucleotide to the second template single-stranded nucleic acid includes releasing the recombinase.

In embodiments, the homologous recombination complex further includes a loading factor, a single-stranded binding (SSB) protein, or both.

In embodiments, the probe oligonucleotide is covalently attached to a substrate.

In embodiments, the probe oligonucleotide is labeled with a first member of a binding pair, and separating the probe-hybridized double-stranded nucleic acid from nucleic acids not hybridized to a probe includes capturing the probe with a second member of the binding pair. In embodiments, (i) the first member of the binding pair is biotin and the second member of the binding pair is avidin or streptavidin, or (ii) the second member of the binding pair is biotin and the first member of the binding pair is avidin or streptavidin.

In embodiments, the double-stranded nucleic acid is a DNA sample. In embodiments, the DNA sample includes genomic DNA. In embodiments, the DNA sample includes picogram quantities of DNA. In embodiments, the DNA sample includes about 1 pg to about 900 pg DNA, about 1 pg to about 500 pg DNA, about 1 pg to about 100 pg DNA, about 1 pg to about 50 pg DNA, about 1 to about 10 pg, DNA, less than about 200 pg, less than about 100 pg DNA, less than about 50 pg DNA, less than about 20 pg DNA, and less than about 5 pg DNA. In other embodiments, the DNA sample includes nanogram quantities of DNA. In embodiments, the DNA sample contains about 1 to about 500 ng of DNA, about 1 to about 200 ng of DNA, about 1 to about 100 ng of DNA, about 1 to about 50 ng of DNA, about 1 ng to about 10 ng of DNA, about 1 ng to about 5 ng of DNA, less than about 100 ng of DNA, less than about 50 ng of DNA less than about 5 ng of DNA, or less that about 2 ng of DNA. In embodiments, the DNA sample includes circulating cell-free DNA (cfDNA). In embodiments, the DNA sample includes microgram quantities of DNA.

In embodiments, the template polynucleotide (and the resulting amplification products) include known adapter sequences on the 5′ and 3′ ends. In embodiments, the template polynucleotide includes known adapter sequences on the 5′ and 3′ ends. In embodiments, the double-stranded amplification products include known adapter sequences on the 5′ and 3′ ends.

In an aspect is provided a method of amplifying a methylated template polynucleotide including: i) contacting a solid support with an annealing solution at a first temperature, wherein the solid support includes a plurality of immobilized primers wherein one or more of the immobilized primers is annealed to a methylated template polynucleotide; ii) contacting the solid support with an extension solution; iii) contacting the solid support with a chemical denaturant at a second temperature, wherein the second temperature is higher than the first temperature; and iv) repeating steps i) to iii) to amplify the methylated template polynucleotide. Thus, in embodiments, amplifying includes a plurality of cycles of strand denaturation, primer hybridization, and primer extension.

In embodiments, prior to contacting the solid support with an extension solution, the method includes contacting the solid support with oxygen. In embodiments, prior to contacting the solid support with a chemical denaturant, the method includes contacting the solid support with oxygen. In embodiments, prior to contacting the solid support with an annealing solution, the method includes contacting the solid support with oxygen.

In embodiments, contacting the solid support with an extension solution occurs at the first temperature and remains in contact with the solid support as the temperature is increased to the second temperature. In embodiments, contacting the solid support with a chemical denaturant occurs at the second temperature and remains in contact with the solid support as the temperature is decreased to the first temperature.

In embodiments, the method further includes removing one or more immobilized primers. In embodiments, following extension of the immobilized primers, the method further includes removing one or more non-extended immobilized primers (i.e., immobilized primers that do not contain an extension of the template polynucleotide or complement template polynucleotide), which may be referred to as unused primers. In embodiments, following amplification of a template polynucleotide, the method includes removing immobilized primers that do not contain a first or second strand (i.e., removing the unused primers).

Methods of removing immobilized primers can include digestion using an enzyme with exonuclease activity (e.g., an exonuclease). In embodiments, removing the immobilized primers includes contacting one or more immobilized primers with an exonuclease. In embodiments, removing the one or mores immobilized primers is performed using an enzyme with 3′-5′ exonuclease activity (e.g., exonuclease I, exonuclease III, exonuclease V, phi29). In embodiments, the enzyme with 3′-5′ exonuclease activity is phi29 polymerase, or a mutant thereof. Removing unused primers may serve to increase the free volume and allow for greater accessibility of the invasion primer. Removal of unused primers may also prevent opportunities for the newly released first strand to rehybridize to an available surface primer, producing a priming site off the available surface primer, thereby facilitating the “reblocking” of the released first strand. In embodiments, the exonuclease is a DNA polymerase, lambda exonuclease, Exo I, Exo III, T5, Exo V, or Exo VII.

In embodiments, the method further includes detecting the amplification products (e.g., the immobilized template polynucleotide and immobilized complementary template polynucleotide). In embodiments, either one or both strands of the amplification products are methylated (e.g., an immobilized methylated template polynucleotide and immobilized methylated complement template polynucleotide) prior to detection. In embodiments, the method further includes sequencing the amplification products. In embodiments, the sequencing includes sequencing-by-synthesis, sequencing-by-binding, sequencing-by-ligation, or pyrosequencing. In embodiments, sequencing includes generating a sequencing read. In embodiments, generating a sequencing read includes executing a plurality of sequencing cycles, each cycle including extending the sequencing primer by incorporating a nucleotide or nucleotide analog using a polymerase and detecting a characteristic signature indicating that the nucleotide or nucleotide analog has been incorporated (e.g., detecting the fluorophore of an incorporated modified nucleotide).

In embodiments, the method further includes sequencing the methylated complement template polynucleotide. In embodiments, sequencing includes incorporating one or more nucleotides into a sequencing primer hybridized to the methylated complement template polynucleotide to generate an extension strand; and detecting the one or more incorporated nucleotides so as to identify each incorporated nucleotide in the extension strand, thereby sequencing the methylated complement template polynucleotide.

In embodiments, the method includes sequencing the first and/or the second strand of a double-stranded amplification product by extending a sequencing primer hybridized thereto. A variety of sequencing methodologies can be used such as sequencing-by-synthesis (SBS), pyrosequencing, sequencing-by-binding, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

In embodiments, generating a first sequencing read or a second sequencing read includes a sequencing-by-binding (see, e.g., U.S. Pat. Pubs. US2017/0022553 and US2019/0048404, each of which is incorporated herein by reference in its entirety). As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′—OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera, CMOS camera, or other suitable detection means).

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

In embodiments, the amplification primer and the sequencing primer includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers (e.g., amplification primer or sequencing primer) include nucleotides ranging from 17 to 30 nucleotides. In embodiments, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

In embodiments, the double-stranded nucleic acid is a cell-free DNA (cfDNA) or circulating tumor DNA (ctDNA). In embodiments, the double-stranded nucleic acid is a cell-free DNA (cfDNA). In embodiments, the double-stranded nucleic acid is a circulating tumor DNA (ctDNA). In embodiments, the double-stranded nucleic acid is from a FFPE sample. In embodiments, the double-stranded nucleic acid is extracted from plasma or from peripheral blood mononuclear cells (PBMCs). In embodiments, the double-stranded nucleic acid is 50 to 100 bp in length. In embodiments, the double-stranded nucleic acid includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA).

In certain embodiments, a method includes annealing a first primer to a 3′-portion of a template described herein, or to a 3′-end of a complementary sequence of a template described herein (e.g., a 3′ end of an amplicon of a template). In certain embodiments, a method includes annealing a first primer to a 3′-portion of a template described herein, where the 3′-portion of the template includes a portion of an adapter (e.g., a first adapter). In certain embodiments, a method includes annealing a first primer to a 3′-portion of a template described herein, where the 3′-portion of the template includes a portion of a Y-adapter. In certain embodiments, a method includes annealing a first primer to a 3′-arm of a Y-adapter of a template described herein, where the 3′-arm of the adapter includes a primer binding site for the first primer. In certain embodiments, a method includes annealing a first primer to a 5′-portion of a second strand of a Y-adapter of a template described herein, where the 5′-portion of the adapter includes a primer binding site for the first primer.

In embodiments where a template includes two hairpin adapters located on opposing sides of a double stranded nucleic acid, a method includes annealing a first primer to a portion of a first adapter of a template described herein. In certain embodiments, a method includes annealing a first primer to a loop of a first hairpin adapter of a template described herein, where the loop of the adapter includes a first primer binding site for the first primer. In some embodiments, a method includes annealing a first primer to a stem of a first hairpin adapter of a template described herein, where the stem of the adapter includes a first primer binding site for the first primer.

In certain embodiments, a method includes sequencing a first portion of a nucleic acid template by extending a first primer, thereby generating a first read including a first nucleic acid sequence of at least a first portion of the double stranded nucleic acid. In some embodiments, a method includes sequencing a reverse strand of a nucleic acid template by extending a first primer, thereby generating a first read including a nucleic acid sequence of at least a portion of the reverse strand of a double stranded nucleic acid.

In certain embodiments, a method includes sequencing a second portion of a nucleic acid template by extending a second primer, thereby generating a second read including a second nucleic acid sequence of at least a second portion of the double stranded nucleic acid. In some embodiments, a method includes sequencing a forward strand of a nucleic acid template by extending a second primer, thereby generating a second read including a nucleic acid sequence of at least a portion of the forward strand of a double stranded nucleic acid. In some embodiments, a method includes annealing a second primer to the nucleic acid template, wherein the second primer includes a sequence that is complementary to a primer binding sequence located within a loop of the hairpin adapter (i.e., second adapter). In certain embodiments, a second primer is annealed to a loop of the hairpin adapter (i.e., second adapter) and a second portion of the nucleic acid template (e.g., the forward strand) is sequenced by extending the second primer, thereby generating a second read of the nucleic acid template.

In some embodiments, a method includes (i) hybridizing a first primer to a 3′-portion of a template where the 3′ portion of the template includes a portion of a Y-adapter, (ii) sequencing a portion of a first strand of a double-stranded nucleic acid, (iii) hybridizing a second primer to a loop or stem of a hairpin adapter of the template, and (iv) sequencing a portion of a second strand of the double-stranded nucleic acid. In some embodiments, the methods herein can be applied to an amplicon or copy of a template (or complement thereof), as well as to the original template.

In some embodiments, the step of sequencing a first portion of a nucleic acid template as described herein is conducted before, after and/or during the step of sequencing a second portion of a nucleic acid template as described herein. For example, in certain embodiments, a second primer is annealed to a loop or stem region of a hairpin adapter and a first portion of a double stranded nucleic acid insert is sequenced by extending the second primer, followed by annealing a first primer to a 3′-end of the template including a portion of a Y-adapter, and sequencing a second portion of the double stranded nucleic acid insert by extending the first primer.

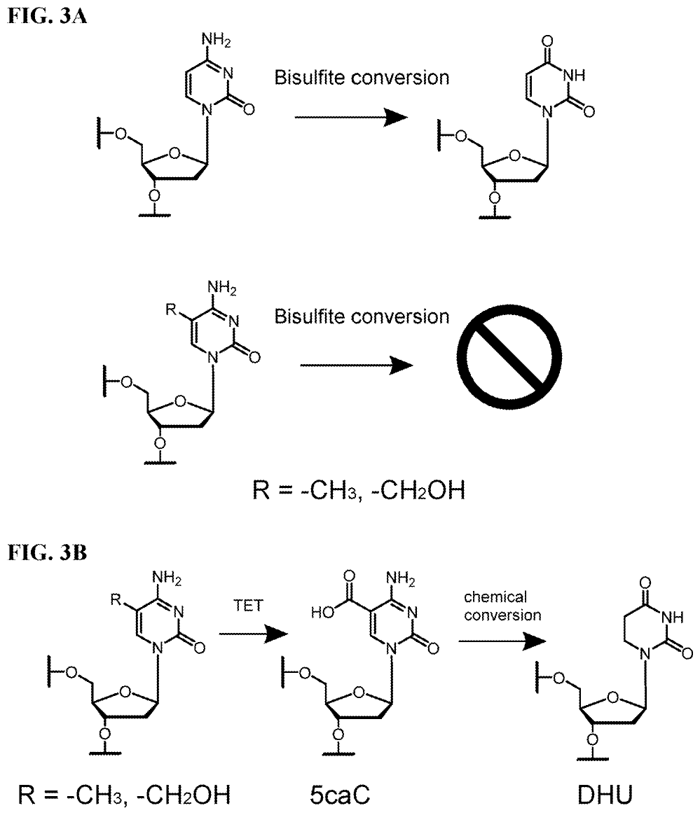

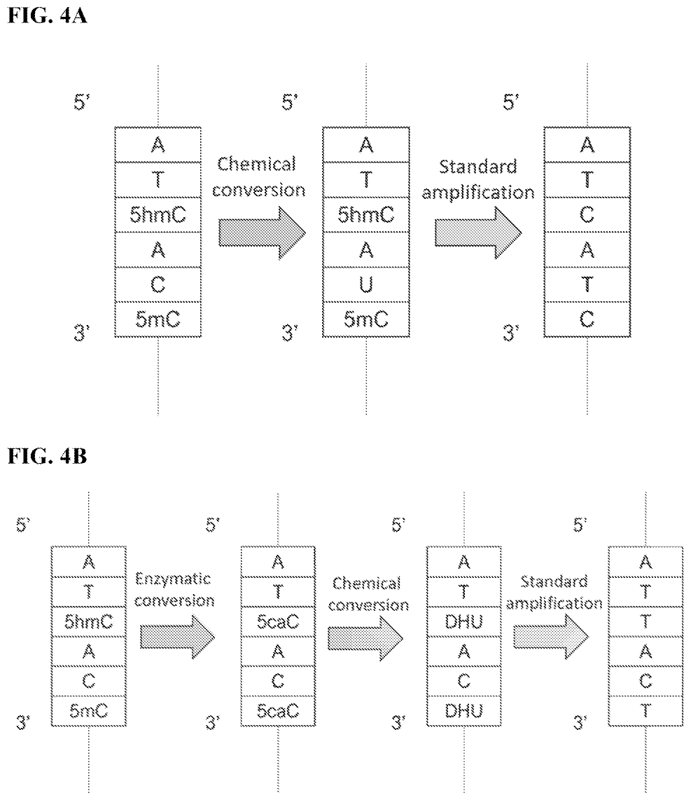

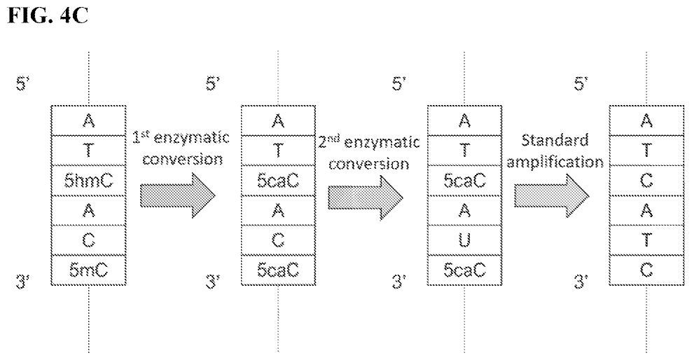

Conversion approaches: chemical approaches. Chemical approaches for converting methylated cytosines have been known for decades. A commonly used agent for modifying unmethylated cytosine preferentially to methylated cytosine is sodium bisulfite. Sodium bisulfite (NaHSO₃) reacts readily with the 5,6-double bond of cytosine, but poorly with methylated cytosine, as described by Olek A., Nucleic Acids Res. 24:5064-6, 1996 or Frommer et al., Proc. Natl. Acad. Sci. USA 89:1827-1831 (1992), each of which is incorporated herein by reference. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil nucleobase (see FIG. 3A and FIG. 4A for additional detail). Alternatively, conversion may be accomplished using restriction enzymes, such as HpaII and MspI, which recognize the sequence CCGG. Uracil is recognized as a thymine by Taq polymerase and other polymerases and therefore upon amplification (e.g., PCR) and subsequently during detection (e.g., sequencing reaction), the resultant product contains cytosine only at the position where 5-methylcytosine occurs in the initial template nucleic acid.

In embodiments, converting one or more cytosine nucleobases of the methylated complement template polynucleotide includes i) contacting the one or more non-methylated cytosine nucleobases with sodium bisulfite to one or more uracil nucleobases and generating a uracil-containing strand.

Conversion approaches: enzymatic approaches. A method for bisulfite-free direct detection of 5-methylcytosine (5mC) and 5-hydroxymethylcytosine (5hmC) has been described (Liu Y et al. Nat. Biotechnol. 2019, 37(4)424-429, which is incorporated herein by reference), which combines ten-eleven translocation (TET) enzymatic oxidation of 5mC and 5hmC to 5-carboxylcytosine (5caC) with pyridine borane reduction of 5caC to dihydrouracil (DHU). Another bisulfite-free approach for methylation analysis is the NEBNext® Enzymatic Methyl-seq product, which first protects 5mC and 5hmC from deamination by TET2 and an oxidation enhancer, followed by APOBEC deamination of unprotected cytosines to uracils.

One unresolved problem with bisulfite conversion is that it is often difficult to distinguish between SNPs or unmethylated cytosine in the same double stranded nucleic acid. This is especially difficult for C>T SNPs, which is the most common substitution (>60%) in human population. Often this requires very high sequencing depth to discern simultaneous SNVs, SNPs, and methylation profiles. For example, computational methods have been developed to predict germline SNPs in bulk sequencing and suggest that at least 30× genomic coverage is required to identify 96% of SNPs from bisulfite converted DNA. Using methods described herein, e.g., generating the Y-template-hairpin constructs, and obtaining sequencing information from both strands permits identification of a SNP and a methylation profile. In embodiments, the methods described herein reduce sequencing overhead with higher accuracy for ctDNA. Methods described herein also differentiate SNV and methylation simultaneously, at low sequencing depths for germline mutations.

In embodiments, the method includes detecting SNVs and methylation status from a double stranded nucleic acid. Although SNVs can determine if a mutation occurred, it cannot reveal tissue of origin. Methylation is highly tissue specific and can be used to predict tissue of origin of cfDNA. Current studies have shown common methylation CpG sites that are differentially methylated depending on tissue. By searching for these different methylation signals within ctDNA, one could determine if there are elevated levels of certain tissue signals within the plasma.

Alternatively, a first modified cytosine nucleobase, such as 5mC or 5hmC, may be enzymatically converted to a second modified nucleobase, such as 5caC utilizing a TET enzyme. In embodiments, the second modified nucleobase, 5caC, may be further converted to dihydrouracil (DHU) following contact with a borane-agent (e.g., pyridine borane). In embodiments, the cytosine nucleobases include unmodified cytosine, 5mC, 5gmC, 5hmC nucleobases, or a combination thereof. In embodiments, the cytosine nucleobases include 5mC, 5gmC, or 5hmC nucleobases. In embodiments, the cytosine nucleobases include 5mC or 5hmC nucleobases. In embodiments, the cytosine nucleobases include 5mC and 5hmC nucleobases.

In embodiments, converting the one or more cytosine nucleobases of the methylated complement template polynucleotide includes contacting the one or more cytosine nucleobases with a ten-eleven translocation (TET) enzyme, an apolipoprotein B mRNA editing enzyme, catalytic polypeptide-like (APOBEC) enzyme, a borane-containing reducing agent, an oxidizing agent, or a combination thereof. In embodiments, the cytosine nucleobases include unmodified cytosine, 5mC, 5gmC, 5hmC nucleobases, or a combination thereof.

In embodiments, converting the one or more cytosine nucleobases of the methylated complement template polynucleotide includes i) contacting the one or more methylated cytosine nucleobases with a ten-eleven translocation (TET) enzyme to generate one or more 5-carboxylcytosine (5caC) nucleobases; and ii) contacting the one or more 5caC nucleobases with borane-containing reducing agent to generate one or more uracil nucleobase analogs.

In embodiments, converting the one or more cytosine nucleobases of the methylated complement template polynucleotide includes i) contacting the one or more cytosine nucleobases with a β-glucosyltransferase to generate one or more β-glucosyl-5-hydroxymethylcytosine (5gmC) nucleobases; ii) contacting the one or more 5gmC nucleobases with a ten-eleven translocation (TET) enzyme to generate one or more 5-carboxylcytosine (5caC) nucleobases; and iii) contacting the one or more 5caC nucleobases with borane-containing reducing agent to generate one or more uracil nucleobase analogs.

In embodiments, converting the one or more cytosine nucleobases of the methylated complement template polynucleotide includes i) contacting the one or more cytosine nucleobases with an oxidizing agent to generate one or more 5-formyl cytosine (5fC) nucleobases; and ii) contacting the one or more 5caC nucleobases with borane-containing reducing agent to generate one or more uracil nucleobase analogs, wherein the oxidizing agent is selected from the group consisting of potassium perruthenate (KRuO₄), Cu(II)/TEMPO (copper(II) perchlorate and 2,2,6,6-tetramethylpiperidine-1-oxyl (TEMPO)), potassium ruthenate, and manganese oxide.

In an embodiment, converting the one or more cytosine nucleobases of the methylated complement template polynucleotide includes i) contacting the one or more methylated cytosine nucleobases with a ten-eleven translocation (TET) enzyme to generate one or more 5-carboxylcytosine (5caC) nucleobases; and ii) contacting the methylated complement template polynucleotide with an apolipoprotein B mRNA editing enzyme, catalytic polypeptide-like (APOBEC) enzyme to generate one or more uracil nucleobases.

In embodiments, the step of converting the 5caC and/or 5fC to DHU includes contacting the DNA sample with a reducing agent including, for example, pyridine borane, 2-picoline borane (pic-BEb), tert-butyl amine borane, borane, sodium borohydride, sodium cyanoborohydride, and sodium triacetoxyborohydride. In embodiments, the reducing agent is pic-BEb and/or pyridine borane.

In embodiments, the converting of one or more unmethylated cytosine nucleobases to a methylated cytosine nucleobase is performed such that CpG dinucleotides within the first extension product are methylated according to the methylation status of the corresponding CpG dinucleotide on the polynucleotide.

In embodiments, the method further includes amplifying the template polynucleotide including one or more cytosine mismatches to generate amplicons including one or more cytosine mismatches.

In certain embodiments, a method includes generating amplicons of the nucleic acid template (e.g., the nucleic acid ligated to a first and second adapter, as described herein). Amplicons may be generated using a suitable amplification method. In certain embodiments, amplicons of a template are generated using a polymerase chain reaction or a rolling circle amplification method, or a combination thereof. In certain embodiments, amplicons are generated using a polymerase chain reaction. In certain embodiments, amplicons are generated using a bridge PCR amplification method. In embodiments, amplicons are generated using thermal bridge polymerase chain reaction (t-bPCR) amplification. In embodiments, amplicons are generated using a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, generating amplicons includes a thermal bridge polymerase chain reaction (t-bPCR) amplification. In embodiments, the plurality of cycles includes thermally cycling between (i) about 85° C. for about 15-30 sec for denaturation, and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer.

Provided herein in an aspect is a method of amplifying a double-stranded nucleic acid template. In embodiments, the method includes (a) ligating a first adapter to a first end of the double stranded nucleic acid, and ligating a second adapter to a second end of the double stranded nucleic acid, wherein the second adapter is a hairpin adapter, thereby forming a nucleic acid template; (b) annealing a first primer to the nucleic acid template, wherein the first primer includes a sequence that is complementary to a portion of the first adapter, or a complement thereof, and is not substantially complementary to a portion of the second adapter; (c) generating amplicons using a suitable amplification method. In embodiments, the method provides a copy of the nucleic acid template as a single-stranded molecule of DNA, and, advantageously, contains both forward and reverse strands of the original double-stranded DNA molecule. In embodiments, the method further includes sequencing the amplicons using a method known in the art or described herein.

In embodiments, the method includes amplifying a double stranded nucleic acid including a first strand and a second strand, the method including: (a) ligating a first adapter to a first end of the double stranded nucleic acid wherein the first adapter is a Y adapter including (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand, and ligating a second adapter to a second end of the double stranded nucleic acid, wherein the second adapter is a hairpin adapter, thereby forming a nucleic acid template; (b) annealing a primer to the nucleic acid template, wherein the first primer includes a sequence that is complementary to a portion of the first adapter, or a complement thereof, and is not substantially complementary to a portion of the second adapter, or a complement thereof; and (c) amplifying the nucleic acid template by extending the primer using a strand-displacing polymerase, thereby generating an amplicon (e.g., a single-stranded amplicon) including a complement of the first and second strand of the double stranded nucleic acid. In embodiments, the amplicon is a contiguous strand of DNA that contains the first and second strand of the double-stranded nucleic acid. In embodiments, the amplicon is a continuous strand lacking free 5′ and 3′ ends. In embodiments, the amplicon is a single-stranded amplicon. In embodiments, after step (a) the method includes amplifying the nucleic acid template to generate a plurality of nucleic acid templates using a polymerase chain reaction.

In embodiments, amplifying the nucleic acid template is on a solid support including a plurality of primers attached to the solid support, wherein the plurality of primers include a plurality of forward primers with complementarity to a complement of the first strand of the Y adapter (e.g., the 5′ arm portion) and a plurality of reverse primers with complementarity to the second strand of the Y adapter (e.g., the 3′ arm portion), and the amplifying includes a plurality of cycles of strand denaturation, primer hybridization, and primer extension, thereby generating a plurality of forward amplicons and a plurality of reverse amplicons.

In embodiments, the plurality of forward primers are covalently attached to the solid support via a first linker and the reverse primers are covalently attached to the solid support via a second linker. The linker tethering the polynucleotide strands may be any linker capable of localizing nucleic acids to arrays. The linkers may be the same, or the linkers may be different Solid-supported molecular arrays have been generated previously in a variety of ways, for example, the attachment of biomolecules (e.g., proteins and nucleic acids) to a variety of substrates (e.g., glass, plastics, or metals) underpins modern microarray and biosensor technologies employed for genotyping, gene expression analysis and biological detection. Silica-based substrates are often employed as supports on which molecular arrays are constructed, and functionalized silanes are commonly used to modify glass to permit a click-chemistry enabled linker to tether the biomolecule.

In embodiments, the method further includes removing the plurality of reverse amplicons, annealing a primer to the amplicon (e.g., the first amplicon), wherein the first primer includes a sequence that is complementary to a portion of the amplicon, or a complement thereof, and sequencing a portion of the first amplicon by extending the primer, thereby generating a sequencing read including a first nucleic acid sequence of at least a first portion of the double stranded nucleic acid. In embodiments, the method further includes removing the plurality of forward amplicons, annealing a primer to the amplicon (e.g., the first amplicon), wherein the first primer includes a sequence that is complementary to a portion of the first amplicon, or a complement thereof, and sequencing a portion of the first amplicon by extending the primer, thereby generating a sequencing read including a first nucleic acid sequence of at least a first portion of the double stranded nucleic acid.

In embodiments, amplifying includes incubation in a denaturant. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO).

In embodiments, amplifying includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In some embodiments, an amplification method includes attaching a nucleic acid template described herein to a substrate. In certain embodiments, attaching a nucleic acid template to a substrate includes annealing a capture nucleic acid to a template. In some embodiments, a capture nucleic acid anneals to a complementary sequence that is present on an adapter portion of a template (e.g., a Y-adapter or hairpin adapter). In certain embodiments, a capture nucleic acid anneals to a primer binding site located on a Y-adapter portion of a template described herein. A capture nucleic acid may anneal to a portion of a Y-adapter on or near the 3′-end or 3′-side of a template. In some embodiments, a capture nucleic acid anneals to a 3′-arm of a Y-adapter on a template.

In embodiments, the nucleic acid template is provided in a clustered array. In embodiments, the clustered array includes a plurality of amplicons localized to discrete sites on a solid support. In embodiments, the solid support is a bead. In embodiments, the solid support is a multiwell container including a plurality of wells, wherein each well includes the immobilized primer(s). In embodiments, the solid support is substantially planar. In embodiments, the solid support is contained within a flow cell. Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes

In some embodiments, an amplification method includes annealing a primer or capture nucleic acid to a portion of a Y-adapter on or near a 3′-end of a template, and extending the primer using a polymerase, thereby generating a first amplicon (first copy) of the template. In certain embodiments, a 3′-end of the first amplicon is annealed to another primer or capture nucleic acid, which is then extended to generate a second amplicon. The amplification process continues until a plurality of first amplicons (e.g., a set of first amplicons) and a plurality of second amplicons (e.g., a set of second amplicons) are generated. In embodiments, a bridge PCR amplification method produces a first set of amplicons that are complementary to an original template, and a second set of amplicons that have nucleic acid sequences substantially identical to the original template, where both the first and second sets of amplicons are attached to a substrate (e.g., a substrate of a flow cell). After bridge amplification, in certain embodiments, the first set of amplicons, or alternatively the second set of amplicons, are removed from a surface or substrate using a suitable method, usually by restriction enzyme. Cleaving one strand may be referred to as linearization. Suitable methods for linearization are known, and described in more detail in U.S. Patent Publication No. 2009/0118128, which is incorporated herein by reference in its entirety. For example, the first strand may be cleaved by exposing the first strand to a mixture containing a glycosylase and one or more suitable endonucleases. In embodiments, cleaving includes chemically cleaving one strand at a cleavable site. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent.

Any suitable enzymatic, chemical, or photochemical cleavage reaction may be used to cleave the cleavage site. The cleavage reaction may result in removal of a part or the whole of the strand being cleaved. Suitable cleavage means include, for example, restriction enzyme digestion, in which case the cleavage site is an appropriate restriction site for the enzyme which directs cleavage of one or both strands of a duplex template; RNase digestion or chemical cleavage of a bond between a deoxyribonucleotide and a ribonucleotide, in which case the cleavage site may include one or more ribonucleotides; chemical reduction of a disulfide linkage with a reducing agent (e.g., THPP or TCEP), in which case the cleavage site should include an appropriate disulfide linkage; chemical cleavage of a diol linkage with periodate, in which case the cleavage site should include a diol linkage; generation of an abasic site and subsequent hydrolysis, etc. In embodiments, the cleavage site is included in the surface immobilized primer (e.g., within the polynucleotide sequence of the primer). In embodiments, one strand of the double-stranded amplification product (or the surface immobilized primer) may include a diol linkage which permits cleavage by treatment with periodate (e.g., sodium periodate). It will be appreciated that more than one diol can be included at the cleavage site. One or more diol units may be incorporated into a polynucleotide using standard methods for automated chemical DNA synthesis. Polynucleotide primers including one or more diol linkers can be conveniently prepared by chemical synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine.

In embodiments, the method includes removing immobilized primers that do not contain a first or second strand of the nucleic acid template (i.e., unused primers) on a solid support. Methods of removing immobilized primers can include digestion using an enzyme with exonuclease activity. Removing unused primers may serve to increase the free volume and allow for greater accessibility. Removal of unused primers may also prevent opportunities for the newly released first strand to rehybridize to an available surface primer, producing a priming site off the available surface primer, thereby facilitating the “reblocking” of the released first strand.

In embodiments, generating the blocking strand includes a plurality of blocking primer extension cycles. In embodiments, generating the blocking strand includes extending the blocking primer by incorporating one or more nucleotides (e.g., dNTPs) using Bst large fragment (Bst LF) polymerase, Bst2.0 polymerase, Bsu polymerase, SD polymerase, Vent exo− polymerase, Phi29 polymerase, or a mutant thereof.

After removal of one of the sets of amplicons from the substrate, the other remaining set of substrate-attached amplicons is subjected to sequencing by annealing a first sequencing primer at the 3′-end (3′-region) of each of the amplicons (formerly a portion of the Y-adapter), and extending the first primer to obtain a sequence read of a 3′ portion of each of the amplicons, which includes a sequence of a first strand of the original double stranded insert. Before, during or after obtaining a sequence read of the 3′-portion of the amplicons, a second primer is annealed to the loop of each of the set of amplicons (i.e., the loop portion of the hairpin adapter used to make the template) and the second primer is used to obtain a second sequence read of a second portion of the amplicon, which includes a sequence of the opposite strand of the original doubled stranded insert. The process described above obtains a sequence read of both strands of the original double stranded nucleic acid insert from a single set of substantially identical amplicons. In some embodiments, sequencing method is complete at this stage and does not require another amplification step. Traditional methods of paired-end sequencing that utilize bridge amplification require a first amplification to obtain a first read of one strand of an insert followed by a second amplification to obtain a second read of the other strand of an insert. The required second amplification step of traditional method introduces a substantial amount of error in the sequencing reads obtained after the second amplification. The methods described herein, in certain embodiments, do not require a second amplification step and therefore provide for less error in the sequence reads obtained. Accordingly, in some embodiments, a method of sequencing both strands of a double stranded nucleic acid, as described herein, includes, or consists essentially of, generating a first read and a second read from the same template. In some embodiments, a method of sequencing both strands of a double stranded nucleic acid, as described herein, includes, or consists essentially of, generating a first read and a second read from a set of amplicons that are substantially complementary to a nucleic acid template. In some embodiments, a method of sequencing both strands of a double stranded nucleic acid, as described herein, includes, or consists essentially of, generating a first read and a second read from a set of amplicons that are substantially identical to a nucleic acid template.

In certain embodiments, a sequencing method provided herein includes sequencing both strands of a double stranded nucleic acid with an error rate of 5×10⁻⁵ or less, 1×10⁻⁵ or less, 5×10⁻⁶ or less, 1×10⁻⁶ or less, 5×10⁻⁷ or less, 1×10⁻⁷ or less, 5×10⁻⁸ or less, or 1×10⁻⁸ or less. In certain embodiments, a sequencing method provided herein includes sequencing both strands of a double stranded nucleic acid with an error rate of 5×10⁻⁵ to 1×10⁻⁸, 1×10⁻⁵ to 1×10⁻⁸, 5×10⁻⁵ to 1×10⁻⁷, 1×10⁻⁵ to 1×10⁻⁷, 5×10⁻⁶ to 1×10⁻⁸, or 1×10⁻⁶ to 1×10⁻⁸. In certain embodiments, a sequencing method provided herein includes sequencing both strands of a double stranded nucleic acid with an error rate of or 1×10⁻⁶ to 1×10⁻⁸. In certain embodiments, a sequencing method provided herein includes sequencing both strands of a double stranded nucleic acid with an error rate of 1×10⁻⁴ to 1×10⁻⁶. In certain embodiments, a sequencing method provided herein includes sequencing both strands of a double stranded nucleic acid with an error rate of 1×10⁻³ or less. In embodiments, a sequencing method provided herein includes sequencing both strands of a double stranded nucleic acid with an error rate of 1×10⁻⁴ or less. In embodiments, a sequencing method provided herein includes sequencing both strands of a double stranded nucleic acid with an error rate of 1×10⁻⁵ or less. In embodiments, a sequencing method provided herein includes sequencing both strands of a double stranded nucleic acid with an error rate of 1×10⁻⁶ or less. In embodiments, a sequencing method provided herein includes sequencing both strands of a double stranded nucleic acid with an error rate of 1×10⁻⁷ or less. In embodiments, a sequencing method provided herein includes sequencing both strands of a double stranded nucleic acid with an error rate of 1×10⁻⁸ or less.

Optionally, after obtaining sequences of both the first strand and second strand of the original double stranded insert from a single set of substantially identical amplicons (e.g., the first set of amplicons) attached to the substrate, a copy of each of the amplicons is generated by a process including annealing the free 3′-end of each amplicon to a surface-bound capture nucleic acid, extending the capture nucleic acid with a polymerase to generate third set of amplicons, removing the first set of amplicons from the substrate, and sequencing the third set of amplicons. In certain embodiments, the novel methods provided herein do not require this second amplification step which introduces additional error into the sequence reads obtained from the third set of amplicons.

In certain embodiments, templates or amplicons described herein are attached to addressable locations on a substrate using a suitable method known in the art or described herein.

In embodiments, a converted template nucleic acid is detected without sequencing. In embodiments, a converted template nucleic acid is detected through the use of a fluorescence-based, real-time PCR method, for example, MethyLight and Digital MethyLight, as described in Campan M. et al. Methods Mol Biol. 2018; 1708: 497-513, which is incorporated herein by reference. MethyLight relies on methylation-specific priming combined with methylation-specific fluorescent probing. Digital MethyLight involves distributing a MethyLight reaction across a 96- or 384-well plate or higher in a microfluidic device, such that the mean initial template DNA concentration is less than one molecule per reaction compartment. Amplification of methylated DNA molecules occurs in a small minority of PCR wells, and therefore represents a digital readout of the original number of template molecules in each sample.

In an aspect is provided a method of detecting a disease in a subject. In embodiments, the method includes obtaining a sample that includes a double-stranded nucleic acid from the subject; identifying whether a disease is present in the sample by sequencing the sample according to the methods described herein, and detecting a disease in a subject when the presence of a disease is identified in the sample. In another aspect is provided a method of diagnosing a subject with a disease. In embodiments, the method includes obtaining a sample that includes a double-stranded nucleic acid from the subject; identifying whether a disease is present in the sample by sequencing the sample according to the methods described herein, and diagnosing a subject with a disease when the presence of a disease is identified in the sample. In some embodiments, the disease is an autoimmune disease, hereditary disease, or cancer.

In embodiments, the disease is an autoimmune disease. In embodiments, the autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, or atopic dermatitis. In embodiments, the autoimmune disease is Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

In embodiments the disease is a hereditary disease. In embodiments, the hereditary disease is cystic fibrosis, alpha-thalassemia, beta-thalassemia, sickle cell anemia (sickle cell disease), Marfan syndrome, fragile X syndrome, Huntington's disease, or hemochromatosis.

In embodiments the disease is a cancer. As used herein, the term “cancer” refers to all types of cancer, neoplasm or malignant tumors found in mammals (e.g., humans), including leukemia, carcinomas and sarcomas. Exemplary cancers that may be treated with a compound or method provided herein include brain cancer, glioma, glioblastoma, neuroblastoma, prostate cancer, colorectal cancer, pancreatic cancer, cervical cancer, gastric cancer, ovarian cancer, lung cancer, and cancer of the head. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof. In embodiments, the cancer is a predefined stage of a breast cancer, a predefined stage of a lung cancer, a predefined stage of a prostate cancer, a predefined stage of a colorectal cancer, a predefined stage of a renal cancer, a predefined stage of a uterine cancer, a predefined stage of a pancreatic cancer, a predefined stage of a cancer of the esophagus, a predefined stage of a lymphoma, a predefined stage of a head/neck cancer, a predefined stage of a ovarian cancer, a predefined stage of a hepatobiliary cancer, a predefined stage of a melanoma, a predefined stage of a cervical cancer, a predefined stage of a multiple myeloma, a predefined stage of a leukemia, a predefined stage of a thyroid cancer, a predefined stage of a bladder cancer, or a predefined stage of a gastric cancer. In some embodiments, the cancer is a predefined subtype of a cancer. In certain instances, the cancer is early stage cancer. In other instances, the cancer is late stage cancer.

In embodiments, the subject is suspected of having a genetic variation or a disease or condition associated with a genetic variation (e.g., an oncogene). In embodiments, the sample, and/or the oncogene includes one or more mutations in one or more of the genes TP53, PIK3CA, PTEN, APC, VHL, KRAS, MLL3, MLL2, ARIDIA, PBRM1, NAV3, EGFR, NF1, PIK3R1, CDKN2A, GATA3, RB1, NOTCH1, FBXW7, CTNNB1, DNMT3A, MAP3K1, FLT3, MALAT1, TSHZ3, KEAP1, CDH1, ARHGAP35, CTCF, NFE2L2, SETBP1, BAP1, NPM1, RUNX1, NRAS, IDH1, TBX3, MAP2K4, RPL22, STK11, CRIPAK, CEBPA, KDM6A, EPHA3, AKT1, STAG2, BRAF, AR, AJUBA, EPPK1, TSHZ2, PIK3CG, SOX9, ATM, CDKN1B, WT1, HGF, KDM5C, PRX, ERBB4, MTOR, TLR4, U2AF1, ARID5B, TET2, ATRX, MLL4, ELF3, BRCA1, LRRK2, POLQ, FOXA1, IDH2, CHEK2, KIT, HIST1H1C, SETD2, PDGFRA, EP300, FGFR2, CCND1, EPHB6, SMAD4, FOXA2, USP9X, BRCA2, NFE2L3, FGFR3, ASXL1, TGFBR2, SOX17, CDKN1A, B4GALT3, SF3B1, TAF1, PPP2RIA, CBFB, ATR, SIN3A, VEZF1, HIST1H2BD, EIF4A2, CDK12, PHF6, SMC1A, PTPN11, ACVR1B, MAPK8IP1, H3F3C, NSD1, TBL1XR1, EGR3, ACVR2A, MECOM, LIFR, SMC3, NCOR1, RPL5, SMAD2, SPOP, AXIN2, MIR142, RAD21, ERCC2, CDKN2C, EZH2, or PCBP1. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. Additional cancer-associated genes include the SEPT9, TMEM106A, NCS1, UXS1, HORMAD2, REC8, DOCK8, or CDKL5 gene.

### III. Compositions & Kits

In an aspect is provided an immobilized polynucleotide including a first nucleic acid sequence including one or more methylated cytosine nucleobases hybridized to a second nucleic acid sequence including one or more uracil nucleobases, wherein the polynucleotide includes one or more cytosine mismatches, and wherein the immobilized polynucleotide is attached to a solid support. In embodiments, the first nucleic acid sequence and the second nucleic acid sequence may be linked together (e.g., with a hairpin adapter). In embodiments, the first nucleic acid sequence and the second nucleic acid sequence are not linked together (e.g., two single-stranded polynucleotides hybridized together, each with free 5′ and 3′ ends).

In an aspect is provided a polynucleotide including a first nucleic acid sequence including one or more methylated cytosine nucleobases hybridized to a second nucleic acid sequence including one or more uracil nucleobases, wherein the polynucleotide includes one or more cytosine mismatches. In embodiments, the first nucleic acid sequence and the second nucleic acid sequence may be linked together (e.g., with a hairpin adapter). In embodiments, the first nucleic acid sequence and the second nucleic acid sequence are not linked together (e.g., two single-stranded polynucleotides hybridized together, each with free 5′ and 3′ ends).

In an aspect is a provided a composition including: (a) a methylation solution including a methyltransferase reagent and methyl donor compound (e.g., a source of methyl groups); (b) an annealing solution including a buffered solution including salts and a chelator; (c) an extension solution including a buffered solution including nucleotides and a polymerase; and (d) a chemical denaturant. In embodiments, the composition further includes a conversion solution including a buffered solution and a cytosine nucleobase converting reagent.

In an aspect is a provided a composition including: (a) a methylation solution including a DNA methyltransferase and methyltransferase ligand (e.g., a source of methyl groups); (b) an annealing solution including a buffered solution including salts and a chelator; (c) an extension solution including a buffered solution including nucleotides and a polymerase; and (d) a chemical denaturant. In embodiments, the composition further includes a conversion solution including a buffered solution and a cytosine nucleobase converting reagent.

In embodiments, the methylation solution includes a DNA methyltransferase. In embodiments, the methylation solution includes a methylation ligand. In embodiments, the methylation solution includes a DNA methyltransferase and a methylation ligand. In embodiments, the methylation solution includes a source of methyl groups (e.g., S-adenosyl-1-methionine (SAM)). In embodiments, the methyltransferase reagent is DNMT1. In embodiments, the DNA methyltransferase (e.g., DNMT1) methylates cytosine residues in hemimethylated DNA in the sequence of 5′ . . . CG . . . 3′. In embodiments, a methyltransferase reagent includes a methyltransferase (e.g., a DNA methyltransferase) and a methyltransferase ligand. In embodiments, a methyltransferase reagent includes a DNA methyltransferase and a methyltransferase ligand. In embodiments, a methyltransferase ligand includes a molecule capable of providing a methyl moiety (e.g., S-adenosyl-1-methionine (SAM)). In embodiments, the methyltransferase reagent is DNMT1, M.SssI, DNMT, or a homolog or mutant thereof. In embodiments, the methyltransferase reagent is DNMT3a, DNMT3b, DRM2, MET1, and CMT3, or a homolog or mutant thereof. In embodiments, the methyltransferase reagent includes DNMT1. In embodiments, the methyltransferase reagent includes DNMT1, M.SssI, DNMT, or a homolog or mutant thereof. In embodiments, the methyltransferase reagent includes DNMT3a, DNMT3b, DRM2, MET1, and CMT3, or a homolog or mutant thereof. In embodiments, the methylation solution, also referred to as a methylation reagent, includes a buffered solution of DNMT1 and SAM. In embodiments, the DNA methyltransferase reagent includes DNMT1. In embodiments, the DNA methyltransferase reagent includes DNMT1, M.SssI, DNMT, or a homolog or mutant thereof. In embodiments, the DNA methyltransferase reagent includes DNMT3a, DNMT3b, DRM2, MET1, and CMT3, or a homolog or mutant thereof. In embodiments, the methylation solution, also referred to as a methylation reagent, is a buffered solution of DNMT1 and SAM. In embodiments, the DNA methyltransferase is DNMT1. In embodiments, the DNA methyltransferase is DNMT1, M.SssI, DNMT, or a homolog or mutant thereof. In embodiments, the DNA methyltransferase is DNMT3a, DNMT3b, DRM2, MET1, and CMT3, or a homolog or mutant thereof.

In certain embodiments, presented herein are compositions for conducting a method described herein, and including one or more elements thereof. In some embodiments, a composition includes (i) a template nucleic acid including sequences of a first strand of a Y-adapter, a forward strand (e.g., a first strand) of the double stranded nucleic acid including one or more modified cytosine nucleobases, a hairpin adapter, a reverse strand (e.g., second strand) of the double stranded nucleic acid and a second strand of the Y-adapter arranged in a 5′ to 3′ direction; wherein the template is attached to a substrate. In embodiments, the composition includes (ii) a primer hybridized to a loop of the hairpin adapter; wherein the template is attached to a substrate. In some embodiments, the substrate is a surface of a flow cell. In some embodiments, the substrate is a polymer coated surface of a flow cell. In embodiments, the substrate is a polymer coated particle (e.g., a polymer coated nanoparticle). In embodiments, the composition includes the complement of the template nucleic acid including sequences of a first strand of a Y-adapter, a forward strand (e.g., a first strand) of the double stranded nucleic acid, a hairpin adapter, a reverse strand (e.g., second strand) of the double stranded nucleic acid and a second strand of the Y-adapter arranged in a 5′ to 3′ direction wherein the complement of the template is attached to a substrate. In embodiments, the substrate includes a glass surface including a polymer coating. In embodiments, the substrate is glass or quartz, such as a microscope slide, having a surface that is uniformly silanized. This may be accomplished using conventional protocols, such as those described in Beattie et a (1995), Molecular Biotechnology, 4: 213. Such a surface is readily treated to permit end-attachment of oligonucleotides (e.g., forward and reverse primers) prior to amplification. In embodiments the substrate surface further includes a polymer coating, which contains functional groups capable of immobilizing primers. In some embodiments, the substrate includes a patterned surface suitable for immobilization of primers in an ordered pattern. A patterned surface refers to an arrangement of different regions in or on an exposed layer of a substrate. For example, one or more of the regions can be features where one or more primers are present. The features can be separated by interstitial regions where capture primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the primers are randomly distributed upon the substrate. In some embodiments, the primers are distributed on a patterned surface.

In an aspect is provided an annealing solution (alternatively referred to herein as a hybridization buffer or hybridization solution). In embodiments, the annealing solution includes an aqueous solution which may contain buffers (e.g., saline-sodium citrate (SSC), tris(hydroxymethyl) aminomethane or “Tris”), aqueous salts (e.g., KCl or (NH₄)₂SO₄)), chelating agents (e.g., EDTA), detergents, surfactants, crowding agents, or stabilizers (e.g., PEG, Tween-20, BSA). In embodiments, the annealing solution includes Tris and is maintained at a pH from about 8.0 to about 9.0. In embodiments, the annealing solution includes Tris, Tris-HCl, Tricine, Bicine, Bis-Tris propane, HEPES, MES, MOPS, MOPSO, BES, TES, CAPS, TAPS, TAPSO, ACES, PIPES, ethanolamine (2-amino methanol; MEA), a citrate compound, and/or a citrate mixture. In embodiments, the annealing solution includes EDTA (ethylenediaminetetraacetic acid), EGTA (ethylene glycol tetraacetic acid), HEDTA (hydroxyethylethylenediaminetriacetic acid), DPTA (diethylene triamine pentaacetic acid), NTA (N,N-bis(carboxymethyl)glycine), citrate anhydrous, sodium citrate, calcium citrate, ammonium citrate, ammonium bicitrate, citric acid, potassium citrate, or magnesium citrate. In some embodiments, the extension solution includes a chelating agent at a concentration of about 0.01-50 mM, or about 0.1-20 mM, or about 0.2-10 mM.

In an aspect is provided an extension solution. In embodiments, the extension solution includes an aqueous solution which may contain buffers (e.g., saline-sodium citrate (SSC), tris(hydroxymethyl)aminomethane or “Tris”), aqueous salts (e.g., KCl or (Mg)₂SO₄)), nucleotides, polymerases, detergents, chelators (e.g., EDTA), surfactants, crowding agents, or stabilizers (e.g., PEG, Tween-20, BSA). In embodiments, the extension solution includes Tris, Tris-HCl, Tricine, Bicine, Bis-Tris propane, HEPES, MES, MOPS, MOPSO, BES, TES, CAPS, TAPS, TAPSO, ACES, PIPES, ethanolamine (2-amino methanol; MEA), a citrate compound, and/or a citrate mixture. In embodiments, the extension solution includes EDTA (ethylenediaminetetraacetic acid), EGTA (ethylene glycol tetraacetic acid), HEDTA (hydroxyethylethylenediaminetriacetic acid), DPTA (diethylene triamine pentaacetic acid), NTA (N,N-bis(carboxymethyl)glycine), citrate anhydrous, sodium citrate, calcium citrate, ammonium citrate, ammonium bicitrate, citric acid, potassium citrate, or magnesium citrate. In some embodiments, the extension solution includes a chelating agent at a concentration of about 0.01-50 mM, or about 0.1-20 mM, or about 0.2-10 mM.

In an aspect is provided a chemical denaturant (e.g., a chemical denaturant as described herein). In embodiments, the chemical denaturant is formamide. In embodiments, the chemical denaturant is NaOH. In embodiments, the method includes contacting the polynucleotide with 100% formamide at a temperature of about 65° C. for about 1-3 minutes, and washing with a reagent including about 50 mM NaCl or equivalent ionic strength and having a pH of about 6.5-8.5.

In embodiments, the chemical denaturant includes formamide, ethylene glycol, sodium hydroxide, or a mixture thereof. In embodiments, the chemical denaturant includes formamide, ethylene glycol, or sodium hydroxide. In embodiments, the chemical denaturant includes formamide. In embodiments, the chemical denaturant is formamide. In embodiments, the chemical denaturant is formamide, and no other chemical denaturants are present. In embodiments, the chemical denaturant is pure formamide. In embodiments, the chemical denaturant is pure formamide, and no other chemical denaturants are present. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO). In embodiments, the denaturant is betaine. In embodiments, the denaturant is dimethyl sulfoxide (DMSO). In embodiments, the denaturant is ethylene glycol. In embodiments, the denaturant is formamide. In embodiments, the denaturant is glycerol. In embodiments, the denaturant is guanidine thiocyanate. In embodiments, the denaturant is 4-methylmorpholine 4-oxide (NMO). In embodiments, the denaturant is betaine, dimethyl sulfoxide (DMSO), ethylene glycol, formamide, glycerol, guanidine thiocyanate, or 4-methylmorpholine 4-oxide (NMO). In embodiments, the denaturant includes an organic diol (e.g., 1,3 propanediol, 1,2-butanediol, 1,3-butanediol, 1,6-hexanediol, 1,2-hexanediol, 2-methyl-2,4-pentanediol), for example 0.01M to about 2.5M organic diol. In embodiments, the denaturant is ethylene glycol, polyethylene glycol, 1,2-propanediol, dimethyl sulfoxide (DMSO), glycerol, formamide, 7-deaza-dGTP, acetamide, betaine or tetramethylammonium chloride (TMAC). The addition of chemical denaturants such as betaine, DMSO, and formamide can be helpful when amplifying GC-rich templates and templates that form strong secondary structures, which can cause DNA polymerases to stall. For example, DMSO and formamide independently are understood to interfere with the formation of hydrogen bonds between the two DNA strands.

In embodiments, the annealing solution and/or the extension solution includes a buffer such as, phosphate buffered saline (PBS), succinate, citrate, histidine, acetate, Tris, TAPS, MOPS, PIPES, HEPES, MES, and the like. The choice of appropriate buffer will generally be dependent on the target pH of the annealing solution and/or the extension solution. In general, the desired pH of the buffer solution will range from about pH 4 to about pH 8.4. In some embodiments, the buffer pH may be at least 4.0, at least 4.5, at least 5.0, at least 5.5, at least 6.0, at least 6.2, at least 6.4, at least 6.6, at least 6.8, at least 7.0, at least 7.2, at least 7.4, at least 7.6, at least 7.8, at least 8.0, at least 8.2, or at least 8.4. In some embodiments, the buffer pH may be at most 8.4, at most 8.2, at most 8.0, at most 7.8, at most 7.6, at most 7.4, at most 7.2, at most 7.0, at most 6.8, at most 6.6, at most 6.4, at most 6.2, at most 6.0, at most 5.5, at most 5.0, at most 4.5, or at most 4.0. Any of the lower and upper values described in this paragraph may be combined to form a range included within the present disclosure, for example, in some instances, the desired pH may range from about 6.4 to about 7.2. Those of skill in the art will recognize that the buffer pH may have any value within this range, for example, about 7.25.

Suitable detergents for use in the annealing solution and/or the extension solution include, but are not limited to, zwitterionic detergents (e.g., 1-Dodecanoyl-sn-glycero-3-phosphocholine, 3-(4-tert-Butyl-1-pyridinio)-1-propanesulfonate, 3-(N,N-Dimethylmyristylammonio)propanesulfonate, 3-(N,NDimethylmyristylammonio) propanesulfonate, ASB-C80, C7BzO, CHAPS, CHAPS hydrate, CHAPSO, DDMAB, Dimethylethylammoniumpropane sulfonate, N,N-Dimethyldodecylamine Noxide, N-Dodecyl-N,N-dimethyl-3-ammonio-1-propanesulfonate, or N-Dodecyl-N,N-dimethyl-3-ammonio-1-propanesulfonate) and anionic, cationic, and non-ionic detergents. Examples of nonionic detergents include poly(oxyethylene) ethers and related polymers (e.g. Brij®, TWEEN®, TWEEN®-20, TRITON®, TRITON X-100 and IGEPAL® CA-630), bile salts, and glycosidic detergents. In embodiments, the annealing solution and/or the extension solution include antioxidants and reducing agents, carbohydrates, BSA, polyethylene glycol, dextran sulfate, betaine, other additives.

In embodiments, the detergent is Triton X-100, Tween 20, Tween 80 or Nonidet P-40. In some embodiments, the detergent includes a zwitterionic detergent such as CHAPS (3-[(3-cholamidopropyl)dimethylammonio]-1-propanesulfonate) or N-Dodecyl-N,N-dimethyl-3-ammonio-1-propanesulfate (DetX). In embodiments, the detergent includes LDS (lithium dodecyl sulfate), sodium taurodeoxycholate, sodium taurocholate, sodium glycocholate, sodium deoxycholate or sodium cholate. In embodiments, the detergent is included at a concentration of about 0.01-0.05%, or about 0.05-0.1%, or about 0.1-0.15%, or about 0.15-0.2%, or about 0.2-0.25%.

In an aspect is provided a wash solution. In embodiments, the wash solution is at a pH from pH 7.5 to pH 9.0. In embodiments, the wash solution includes a chelator. In embodiments, the wash solution includes a surfactant. In embodiments, the wash includes Tris-HCl, pH 8.5, containing SDS, EDTA, and NaCl. The wash solution can include SSC (e.g., at any concentration of about 1-5×) and a detergent (e.g., Tween-20 or Triton X-100).

In embodiments, the wash solution includes Tris, Tris-HCl, Tricine, Bicine, Bis-Tris propane, HEPES, MES, MOPS, MOPSO, BES, TES, CAPS, TAPS, TAPSO, ACES, PIPES, ethanolamine (2-amino methanol; MEA), a citrate compound, a citrate mixture, NaOH and/or KOH. In embodiments, the pH buffering agent can be present in the wash solution at a concentration of about 1-100 mM, or about 10-50 mM, or about 10-25 mM. In embodiments, the pH of solutions described here in can be adjusted to a pH of about 4-9, or a pH of about 5-9, or a pH of about 5-8.

In embodiments, the metal chelating agent (i.e., a chelator) in the wash solution includes EDTA (ethylenediaminetetraacetic acid), EGTA (ethylene glycol tetraacetic acid), HEDTA (hydroxyethylethylenediaminetriacetic acid), DPTA (diethylene triamine pentaacetic acid), NTA (N,N-bis(carboxymethyl)glycine), citrate anhydrous, sodium citrate, calcium citrate, ammonium citrate, ammonium bicitrate, citric acid, potassium citrate, or magnesium citrate. In some embodiments, the wash solution includes a chelating agent at a concentration of about 0.01-50 mM, or about 0.1-20 mM, or about 0.2-10 mM.

In some embodiments, the salt in the wash solution includes NaCl, KCl, NH2SO4 or potassium glutamate. In some embodiments, the detergent includes an ionic detergent such as SDS (sodium dodecyl sulfate). The wash solution can include a monovalent salt at a concentration of about 25-500 mM, or about 50-250 mM, or about 100-200 mM. In embodiments, the detergent in the wash solution includes a non-ionic detergent such as Triton X-100, Tween 20, Tween 80 or Nonidet P-40. In embodiments, the detergent includes a zwitterionic detergent such as CHAPS (3-[(3-cholamidopropyl)dimethylammonio]-1-propanesulfonate) or N-Dodecyl-N,N-dimethyl-3-ammonio-1-propanesulfate (DetX). In some embodiments, the detergent includes LDS (lithium dodecyl sulfate), sodium taurodeoxycholate, sodium taurocholate, sodium glycocholate, sodium deoxycholate or sodium cholate. In some embodiments, the detergent is included in the wash solution at a concentration of about 0.01-0.05%, or about 0.05-0.1%, or about 0.1-0.15%, or about 0.15-0.2%, or about 0.2-0.25%. In embodiments, the wash solution includes SSC (e.g., at any concentration of about 1-5×) and a detergent (e.g., Tween-20 or Triton X-100).

In an aspect is provided a kit containing the component necessary to perform the methods as described herein, including embodiments. Generally, the kit includes one or more containers providing a composition, and one or more additional reagents (e.g., a buffer suitable for polynucleotide extension). The kit may also include a template nucleic acid (DNA and/or RNA), one or more primer polynucleotides, nucleotides (including, e.g., deoxyribonucleotides, ribonucleotides, labeled nucleotides, and/or modified nucleotides), buffers, salts, and/or labels (e.g., fluorophores). In embodiments, the kit further includes instructions. In embodiments the kit includes one or more enclosures (e.g., boxes, bottles, or cartridges) containing the relevant reaction reagents and/or supporting materials.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol τ DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g., Therminator γ, 9° N polymerase (exo−), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, Bst polymerase (e.g., Bst Lf), phi29 mutant polymerase or a thermostable phi29 mutant polymerase. In embodiments, the kit includes a modified terminal deoxynucleotidyl transferase (TdT) enzyme.

In embodiments, the solid support includes a plurality of immobilized oligonucleotides (e.g., immobilized primers, such as immobilized forward and immobilized reverse primers, or immobilized first and immobilized second primers) attached to the solid support via a linker. Additional examples of immobilized oligonucleotides include, for example, an immobilized non-methylated complement template polynucleotide, an immobilized methylated complement template polynucleotide, a plurality of immobilized methylated polynucleotides, an immobilized uracil-containing polynucleotide, an immobilized complement of the immobilized methylated complement template polynucleotide, or a complement thereof. In embodiments, the methylated template polynucleotide and methylated complement template polynucleotide are covalently attached to the solid support. In embodiments, the 5′ end of the template and complement template polynucleotides contains a functional group that serves to tether the template and complement template polynucleotides to the solid support (e.g., a bioconjugate linker). Non-limiting examples of covalent attachment include amine-modified polynucleotides reacting with epoxy or isothiocyanate groups on the solid support, succinylated polynucleotides reacting with aminophenyl or aminopropyl functional groups on the solid support, dibenzocycloctyne-modified polynucleotides reacting with azide functional groups on the solid support (or vice versa), trans-cyclooctyne-modified polynucleotides reacting with tetrazine or methyl tetrazine groups on the solid support (or vice versa), disulfide modified polynucleotides reacting with mercapto-functional groups on the solid support, amine-functionalized polynucleotides reacting with carboxylic acid groups on the core via 1-ethyl-3-(3-dimethylaminopropyl)-carbodiimide hydrochloride (EDC) chemistry, thiol-modified polynucleotides attaching to a solid support via a disulfide bond or maleimide linkage, alkyne-modified polynucleotides attaching to a solid support via copper-catalyzed click reactions to azide functional groups on the solid support, and acrydite-modified polynucleotides polymerizing with free acrylic acid monomers on the solid support to form polyacrylamide or reacting with thiol groups on the solid support. In embodiments, the primer is attached to the solid support polymer through electrostatic binding. For example, the negatively charged phosphate backbone of the primer may be bound electrostatically to positively charged monomers in the solid support.

In embodiments, the solid support (alternatively referred to as a substrate) includes a silica surface including a polymer coating. In embodiments, the solid support includes a glass surface including a polymer coating. In embodiments, the substrate is silica or quartz, such as a microscope slide, having a surface that is uniformly silanized. This may be accomplished using conventional protocols, such as those described in Beattie et al (1995), Molecular Biotechnology, 4: 213. Such a surface is readily treated to permit end-attachment of oligonucleotides (e.g., forward and reverse primers) prior to amplification. In embodiments the substrate surface further includes a polymer coating, which contains functional groups capable of immobilizing primers. In some embodiments, the substrate includes a patterned surface suitable for immobilization of primers in an ordered pattern. A patterned surface refers to an arrangement of different regions in or on an exposed layer of a substrate. For example, one or more of the regions can be features where one or more primers are present. The features can be separated by interstitial regions where capture primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the primers are randomly distributed upon the substrate. In some embodiments, the primers are distributed on a patterned surface. In embodiments, the solid support includes a particle having a surface that includes a polymer coating. In embodiments, the immobilized primers are immobilized to the polymer coated particle. In embodiments, the polymer coated particles are themselves immobilized on a planar substrate. In embodiments, the solid support includes a discrete particle. In embodiments, the solid support includes a nanoparticle. In embodiments, the solid support is a bead. In embodiments, the solid support is a multiwell container including a plurality of wells, wherein each well includes the immobilized primer(s).

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the kit includes an annealing solution, an extension solution, and a chemical denaturant.

In an aspect is provided a microfluidic device, wherein the microfluidic device is capable of performing any of the methods described herein, including embodiments. The microfluidic device is applicable for amplifying, processing, and/or detecting samples of analytes of interest in a flow cell. Within this application the fluidic system is made in reference to nucleic acid sequencing (i.e., a genomic instrument) which allows for the sequencing of nucleic acid molecules. However, the techniques disclosed herein may be applied to any system making use of reaction vessels, such as flow cells, for detection of analytes of interest, and into which solutions are introduced during preparation, reaction, detection, or any other process on or within the reaction vessel. The term “microfluidic device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. In embodiments, the device includes a light source that illuminates a sample, an objective lens, and a sensor array (e.g., complementary metal-oxide-semiconductor (CMOS) array or a charge-coupled device (CCD) array). Nucleic acid sequencing devices may further include valves, pumps, and specialized functional coatings on interior walls. For example, the microfluidic device is a nucleic acid sequencing device provided by Singular Genomics™ (e.g., the G4™ sequencer), Illumina™, Inc. (e.g. HiSeg™, MiSeg™, NextSeq™, or NovaSeq™ systems), Life Technologies™ (e.g. ABI PRISM™, or SOLiD™ systems), Pacific Biosciences (e.g. systems using SMRT™ Technology such as the Sequel™ or RS II™ systems), or Qiagen (e.g. Genereader™ system).

### EXAMPLES

### Example 1. Solid Phase Amplification with Methylation Retention

Beyond somatic mutations, epigenetic information, such as biomolecule methylation, and/or additional protein biomarkers combined with cell-free DNA (cfDNA) and circulating-tumor DNA (ctDNA) analyses are useful in determining tumor origin at an early stage. Biomolecule methylation, such as DNA methylation, is widespread and plays a critical role in the regulation of gene expression in development, differentiation, and disease. Methylation is an epigenetic modification in which a methyl group is added to cytosines and/or adenine nucleobases, and frequently occurs in regions of DNA where a cytosine nucleotide is followed by a guanine nucleotide in the linear sequence of bases along its 5′→3′ direction, referred to as a CG or CpG site. In mammals, cytosines in CpG dinucleotides can be methylated to form 5-methylcytosine. Methylating the cytosine within a gene can change its expression, normally results in transcriptional silencing or suppression. In mammals, 70% to 80% of CpG cytosines are methylated and a total number of 28 million CpG sites exist in human. Mammalian DNA methylation of cytosines within the CpG dinucleotide context has been found to be associated with a number of key processes including embryogenesis, genomic imprinting, X-chromosome inactivation, aging, and carcinogenesis. In embryogenesis, DNA methylation patterns are largely erased and then re-established between generations in mammals. Almost all of the methylations from the parents are erased, first during gametogenesis, and again in early embryogenesis, with demethylation and remethylation occurring each time. In many disease processes, such as cancer, gene promoter CpG islands acquire abnormal hypermethylation, which results in transcriptional silencing that can be inherited by daughter cells following cell division. In particular regions of genes, for example gene promoter regions, an increase in cytosine methylation at gene promoter regions can inhibit the expression of these genes (Robertson K D Nat. Rev. Genet. 6, 597-610 (2005)). The gene silencing effect of methylated regions is accomplished through the interaction of methylcytosine binding proteins with other structural components of the chromatin, which, in turn, makes the DNA inaccessible to transcription factors through histone deacetylation and chromatin structure changes (Greenberg MVC and Bourc'his D Nat. Rev. Mol. Cell Biol. 20, 590-607 (2019)). Cancers take advantage of this mechanism, and hypermethylate genomic regions associated with DNA repair genes.

Methylation patterns also play an important role in genomic imprinting, in which imprinted genes are preferentially expressed from either the maternal or paternal allele. Patterns of methylation in a genome are heritable because of the semi-conservative nature of DNA replication. During this process, the daughter strand, newly replicated on a methylated template strand is not initially methylated, but the template strand directs methyltransferase enzymes to fully methylate both strands. Deregulation of imprinting has been implicated in several developmental disorders. Moreover, there is abundant evidence that aberrant DNA methylation can preclude normal development.

There are around 25,000 CpG islands in the human genome. CpG islands are usually understood as polynucleotide regions with a length greater than 200 bp having GC content greater than 50%. In various cancers such as leukemia, it has been previously reported that there is a global decrease in DNA methylation and an increase in methylation specifically at CpG islands. About 40% of human genes do not contain bona fide CpG islands in their promoter. While CpG island promoters make up 55-75% of all transcription start sites in vertebrate genomes, only a small fraction are targeted by DNA hypermethylation in cancer (Zheng Y et al. Nat. Commun. 2021; 12(1): 2485). Cytosine methylation can attract methylated DNA binding proteins and histone deacetylases to methylated CpG islands during chromatin compaction and gene silencing. In an unmethylated state, cytosine is converted to uracil after deamination, which is recognized by the cell's repair machinery and is removed, while in a methylated state deamination of cytosine results in the formation of thymine which is not recognized by the repair machinery. Therefore, the presence or absence of hypermethylation at these CpG islands can be used to detect tumor cells. Certain transcription factors such as c-Myc do not bind to their recognition sequences in the methylated state, suggesting that inappropriate methylation of its recognition sites could have profound implications on the cancer epigenome (Jones P A and Baylin S B. Cell. 2007; 128: 683-692). As cancer cells are constantly evolving to avoid treatment regimens, there is a need for a method to detect a tumor cell with high accuracy.

The local sequence information around a methylation site has been shown to have an impact on cancer development Retaining the methylation information is critical for providing highly accurate sequence information that is representative of the original biological source and related context. For example, it has been observed that sequence motifs that are protein to have a hypermethylated profile are GC-rich with respect to others (e.g., CpG island methylator phenotype), and has been reported in several cancers, leading to the hypothesis of an epigenetic-driven onset of malignant transformation affecting gene expression. Specifically, it was found that several CpG island enriched motifs in stomach adenocarcima (STAD) and prostate adenocarcinoma (PRAD) are mainly localized within the 200 bp surrounding the transcription start site, suggesting possible direct involvement on corresponding gene expression activity (see, Scala, G et al. Scientific Reports. 2020; 10:1721, which is incorporated herein by reference in its entirety). Additionally, studies have highlighted the effects of methylation on the local DNA structural conformation in cancer. For example, the RAS oncogenes are hypomethylated in cancer, with KRAS being the most frequently mutated of the three proteins. Studies into the effects of the local sequence around codons 12, 13, and 14 of KRAS indicated that the 5′ thymine base which precedes codon 12, which is one of the most frequently mutated sites, contributes greatly to the level of structural distortion upon mutation to a cytosine, and even more as a methylated cytosine (see, Menzies G E et al. BMC Chemistry. 2021; 15:51, which is incorporated herein by reference in its entirety). Increased distortion in a given region of DNA may influence repair efficiencies (e.g., nucleotide excision repair) and the prevalence/frequency of mutations.

A common method of determining the methylation level and/or pattern of DNA requires methylation status-dependent conversion of cytosine in order to distinguish between methylated and non-methylated CpG dinucleotide sequences. For example, bisulfite conversion is a process in which genomic DNA is denatured (i.e., rendered single-stranded) and treated with sodium bisulfite, leading to deamination of unmethylated cytosine nucleobases into uracil nucleobases, while methylated cytosine nucleobases (e.g., 5-methylcytosine and 5-hydroxymethylcytosine) remain unchanged. Thus, thymine nucleobases detected in bisulfite sequencing correspond to either thymine nucleobases or unmethylated cytosine nucleobases in the original DNA, and alignment with the original template sequence easily differentiates between them. One consequence of bisulfite-mediated deamination of cytosine is that the bisulfite treated cytosine is converted to uracil, which reduces the complexity of the genome. Specifically, a typical 4-base genome (A,T,C,G) is essentially reduced to a 3-base genome (A,T,G) because uracil is read as thymine during downstream analysis techniques such as PCR and sequencing reactions. Thus, the only cytosines present are those that were methylated prior to bisulfite conversion. Because the complexity of the genome is reduced, standard methods for comparing and/or aligning a bisulfite-converted sequence to the pre-conversion genome can be cumbersome and, in some cases, ineffective. For example, problems may arise when aligning converted fragments to the genome, especially when using short sequences.

While bisulfite conversion is the current standard for performing DNA methylation analysis, it is a harsh chemical reaction which can lead to DNA degradation, severely limiting its utility if sample DNA quantities are low, as is often the case with cfDNA. Additionally, the complete conversion of unmodified cytosine to thymine reduces sequencing complexity, potentially leading to poor sequencing quality, low mapping rates, and uneven genome coverage. An alternative method for bisulfite-free detection of modified cytosine nucleobases (e.g., 5-methylcytosine (5mC) and 5-hydroxymethylcytosine (5hmC)) has been described (Liu Y et al. Nat. Biotechnol. 2019, 37(4)424-429, which is incorporated herein by reference), which utilizes an enzymatic approach, and combines the ten-eleven translocation (TET) oxidation of 5mC and 5hmC to 5-carboxylcytosine (5caC) with pyridine borane reduction of 5caC to dihydrouracil (DHU). Subsequent PCR converts DHU to thymine, enabling a C-to-T transition of 5mC and 5hmC. This TET-assisted pyridine borane sequencing (TAPS) method results in higher mapping rates and more even coverage than bisulfite conversion and may be applied to the methods described herein for linked duplex methylation profiling. Another bisulfite-free approach for methylation analysis is the NEBNext® Enzymatic Methyl-seq product (EM-Seq), which first protects 5mC and 5hmC from deamination by TET2 and an oxidation enhancer, followed by APOBEC deamination of unprotected cytosines to uracils. However, these enzymatic methods require single stranded template nucleic acids (i.e., denaturing the double stranded template nucleic acid). As described herein, it is advantageous to retain the double stranded information of the original double stranded template nucleic acid.

It has been recognized that an accurate genome methylation analysis is dependent on the maintenance of methylation information during the processing of the DNA, such as DNA in minute amounts or DNA from a single cell or cell free DNA. Provided herein are methods for amplifying DNA to produce amplicons having the methylation information or status of the original template DNA. These methods further enable the study of DNA methylation in connection with cancer diagnosis by comparing the methylation status of a DNA sample from an individual, such as a cell free DNA sample obtained from blood, with the methylation status of DNA indicating cancer, i.e. a standard. For example, if the methylation status of the DNA sample correlates with the standard methylation status indicating cancer, then the individual is diagnosed with cancer. Methylation patterns for cancer DNA that may serve as a standard in the methods described herein are known to those of skill in the art as described in Vadakedath S, Kandi V (2016) DNA Methylation and Its Effect on Various Cancers: An Overview. J Mol Biomark Diagn S2:017. doi: 10.4172/2155-9929.S2-017 and A DataBase of Methylation Analysis on different type of cancers: MethHC: a database of DNA methylation and gene expression in human cancer. W. Y. Huang, S. D. Hsu, H. Y. Huang, Y. M. Sun, C. H. Chou, S. L. Weng, H. D. Huang Nucleic Acids Res. 2015 January; 43(Database issue):D856-61 each of which is hereby incorporated by reference in its entirety.

As described above, both chemical (e.g., bisulfite) and enzymatic (e.g., TET enzyme) conversion strategies reduce the complexity of the amplified library when the converted nucleobase, for example uracil, is amplified and detected as thymine. Standard thermal PCR typically includes thermal changes (e.g., increasing the temperature to denature double-stranded DNA (dsDNA) and decreasing the temperature to anneal a primer) resulting in cycling between denaturation, annealing, and extension conditions to generate amplification products. First the temperature is increased (e.g., temperature is increased to 98° C.) to denature input dsDNA to generate single-stranded DNA (ssDNA). Next, the temperature is reduced (e.g., temperature is reduced to 45° C.) to permit annealing of an amplification primer. Finally, the temperature is increased to the operating temperature (e.g., 72° C.) to enable a polymerase to extend the annealed primer and generate a copy of the ssDNA. This process is repeated to generate a sufficient number of copies of the input nucleic acid.

Unfortunately, merely adding a DNA methyltransferase during PCR cycling does not retain the methylation information of the template nucleic acid within the amplification products. Methyltransferases have a central role in epigenetic gene regulation. DNA methyltransferases, for example DNMT1, use S-adenosylmethionine as a methyl group donor to catalyze DNA methylation, which occurs primarily at the 5-carbon of cytosine, forming 5-methylcytosine. DNMT1 is the most abundant DNA methyltransferase in mammalian cells and is considered to be the key maintenance methyltransferase due to its ability to predominantly methylate hemimethylated CpG di-nucleotides in the mammalian genome. This enzyme is 7 to 100-fold more active on hemimethylated DNA as compared with the unmethylated substrate in vitro. DNA methyltransferases such as DNMT1 typically have an optimal reaction temperature of 37° C. and are inactivated upon incubation at elevated temperatures (e.g., 65° C. or higher). In the case of conventional PCR, using a methyltransferase reagent (e.g., DNMT1) during amplification is not possible because thermal denaturation would denature the methyltransferase enzyme, which is not heat-tolerant at the operating temperature for polymerase extension (e.g., 72° C.).

Other isothermal amplification methods, for example, those in which polymerase extension and methyltransferase targeting of hemimethylated DNA for cytosine methylation would occur at the same time, have additional limitations. DNMT1, for example, is not able to methylate hemimethylated CpG sites on different strands of DNA in a processive manner, and methylates DNA on only one strand of the DNA (see, Hermann A et al. J. Biol. Chem. 2004; 279(46); 48350-9, which is incorporated herein by reference in its entirety). In such isothermal protocols, both methylation and extension would need to proceed in a way such that polymerase extension of the primers is not faster than methyltransferase activity, as once complementary strands are made without methylation transfer, the amplification is biased towards loss of methylation. Importantly, during an isothermal protocol, the methyltransferase and polymerase extension steps are not performed at their optimal temperature and buffer conditions, further limiting optimal enzyme activity in the reaction mixture.

The solid-phase amplification techniques described herein (e.g., chemical bridge PCR or cbPCR) are well-suited for performing methods of DNA amplification while retaining methylation patterns of the initial template nucleic acid (e.g., using a methyltransferase reagent during cbPCR). For example, cbPCR facilitates cyclic amplification, enabling temperature and fluidic variability, and therefore allowing the use of a methyltransferase on a solid support at 37° C. Once cytosine methylation at hemimethylated CpG sites has been performed, the methyltransferase reagent can be exchanged for the denaturant and polymerase reagent mixtures as the amplification cycle proceeds, wherein each of the denaturation and polymerase extension steps typically occurs at elevated temperatures (e.g., about 50° C. to about 65° C.). The solid-phase amplification protocol provides a key advantage over traditional PCR in that the temperature can be adjusted to optimal levels for each specific reaction mixture that is introduced, in addition to being amenable to automated fluidic manipulation.

Using methods and/or compositions described herein, the complexity of the target nucleic acids is preserved by keeping track of complementary strands after being subjected to, for example, bisulfite conversion of nucleic acids. In order to preserve complexity of the nucleic acid, embodiments of the present invention relate to a pairing of the cytosine-converted sequences of both strands of a double-stranded nucleic acid and using the sequence information from both strands to determine the sequence and/or methylation status of one or both strands prior to conversion. In alternate aspects, methods are described for retaining methylation information of non-paired strand template nucleic acids (e.g., templates that include Y-shaped adapters at both ends) during solid-phase amplification.

### Example 2. Linked Duplex Sequencing: Cytosine Conversion with Methylation Retention

Methylation of CpG dinucleotide sequences can be measured by employing cytosine conversion-based technologies. Commonly used bisulfite conversion modifies unmethylated cytosine nucleobases to uracil nucleobases. Sodium bisulfite (NaHSO₃) reacts readily with the 5,6-double bond of cytosine, but poorly with methylated cytosine, as described by Olek A., Nucleic Acids Res. 24:5064-6, 1996 or Frommer et al., Proc. Natl. Acad. Sci. USA 89:1827-1831 (1992), each of which is incorporated herein by reference. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by Taq polymerase and other polymerases and therefore upon PCR or during a sequencing reaction, the resultant product contains cytosine only at the position where 5-methylcytosine occurs in the starting template nucleic acid (see, e.g., FIG. 4A). Alternatively, conversion may be accomplished using restriction enzymes, such as HpaII and MspI, which recognize the nucleotide sequence CCGG.

Traditionally, the recovery of bisulfite-converted DNA is limited due to DNA degradation caused by extended-duration sodium bisulfite treatment protocols and subsequent depyrimidation (Grunau C et al. Nucleic Acids Res. 2001, 29(13):E65-5). Optimized bisulfite conversion protocols that include a fast deamination step reduce incubation times from 12 to 16 hours to 40 min by using a highly concentrated bisulfite solution at high temperatures, leading to a more homogenous conversion of cytosine due to the easier process of DNA denaturation at high temperatures and reduced degradation due to shorter incubation times (Shiraishi M and Hayatsu H. DNA Res. 2004, 11(6):409-15). One study has shown that bisulfite treatment of cfDNA for 30 min at 70° C. leads to complete conversion of cytosine to uracil and is achieved with high post-treatment DNA recovery (Yi S et al. BMC Molecular Biol. 2017, 18:24, which is incorporated herein by reference). Such rapid-bisulfite conversion can also be used in the method described herein. For example, 10 M (NH₄) HSO₃—NaHSO₃ bisulfite solution was added to Y-template-hairpin constructs. The mixtures are heated for 30 min at 70° C. or for 10 min at 90° C. and subsequently cooled to 4° C.

### Linked Duplex Sequencing: Library Prep Overview

Commercially available next-generation sequencing (NGS) technologies typically require library preparation, whereby a pair of specific adapter sequences are ligated to the ends of DNA fragments in order to enable sequencing by the instrument. Typically, preparation of a nucleic acid library involves 5 steps: DNA fragmentation, polishing, adapter ligation, size selection, and library amplification.

Fragmentation of DNA can be achieved by enzymatic digestion or physical methods (e.g., sonication, nebulization, or hydrodynamic shearing). Enzymatic digestion produces DNA ends that can be efficiently polished and ligated to adapter sequences. However, it is difficult to control the enzymatic reaction and produce fragments of predictable length. In addition, enzymatic fragmentation is frequently base-specific thus introducing representation bias into the sequence analysis. Alternatively, physical methods to fragment DNA are random and DNA size distribution can be more easily controlled, but DNA ends produced by physical fragmentation are often damaged and a conventional polishing reaction may be insufficient to generate ample ligation-compatible ends. Typical polishing mixtures contain T4 DNA polymerase and T4 polynucleotide kinase. These enzymes excise 3′ overhangs, fill in 3′ recessed ends, and remove any potentially damaged nucleotides thereby generating blunt ends on the nucleic acid fragments. The T4 polynucleotide kinase used in the polishing mix adds a phosphate to the 5′ ends of DNA fragments that can be lacking such, thus making them ligation-compatible to NGS adapters.

Prior to ligation, adenylation of repaired nucleic acids using a polymerase which lacks 3′-5′ exonuclease activity is often performed in order to minimize chimera formation and adapter-adapter (dimer) ligation products. In these methods, single 3′ A-overhang DNA fragments are ligated to single 5′ T-overhang adapters, whereas A-overhang fragments and T-overhang adapters have incompatible cohesive ends for self-ligation. During size selection, fragments of undesired sizes are eliminated from the library using gel or bead-based selection in order to optimize the library insert size for the desired sequencing read length. This often maximizes sequence data output by minimizing overlap of paired end sequencing that occurs from short DNA library inserts. Amplifying libraries prior to NGS analysis is typically a beneficial step to ensure there is a sufficient quantity of material to be sequenced.

### Linked Duplex Sequencing: Ligating Adapters

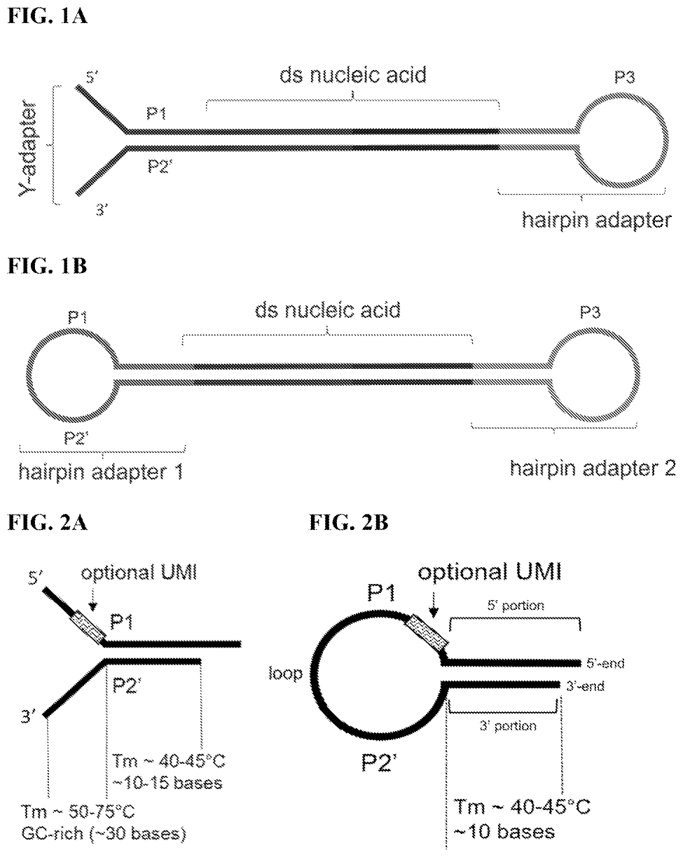

In some aspects of a method herein, an adapter-target-adapter nucleic acid template (FIG. 1A and FIG. 1B) is provided where two non-identical adapters are ligated to each respective end of a polynucleotide duplex. Embodiments of adapters contemplated herein include those shown in FIGS. 2A-2D. A polynucleotide duplex refers to a double-stranded portion of a polynucleotide, for example a polynucleotide desired to be sequenced.

As depicted in FIG. 1A, a first adapter is a Y adapter (alternatively, this may be referred to as a mismatched adapter or a forked adapter) that is ligated to one end of a polynucleotide duplex. The adapter is formed by annealing two single-stranded oligonucleotides, herein referred to as P1 and P2′. P1 and P2′ may be prepared by a suitable automated oligonucleotide synthesis technique. The oligonucleotides are partially complementary such that a 3′ end and/or a 3′ portion of P1 is complementary to the 5′ end and/or a 5′ portion of P2′. A 5′ end and/or a 5′ portion of P1 and a 3′ end and/or a 3′ portion of P2′ are not complementary to each other, in certain embodiments. When the two strands are annealed, the resulting Y adapter is double-stranded at one end (the double-stranded region) and single-stranded at the other end (the unmatched region), and resembles a ‘Y’ shape.

The single-stranded portions (the unmatched regions) of both P1 and P2′ have an elevated melting temperature (Tₘ) (e.g., about 75° C.) relative to their respective complements to enable efficient binding of surface primers and stable binding of sequencing primers. To achieve an elevated Tₘ in a reasonable length primer, the GC content is often >50% (e.g., approximately 60-75% GC content). In contrast to the single-stranded portions, a double-stranded region, in certain embodiments, has a moderate Tₘ (e.g., 40-45° C.) so that it is stable during ligation. In embodiments, a double-stranded region has an elevated Tₘ (e.g., 60-70° C.). In embodiments, the GC content of the double-stranded region is >50% (e.g., approximately 60-75% GC content). The unmatched region of P1 and P2′, in certain embodiments, are about 25-35 nucleotides (e.g., 30 nucleotides), whereas the double-stranded region is shorter, ranging about 10-20 nucleotides (e.g., 13 nucleotides) in total. For example, P2′ may be a total of 43 nucleotides in length, as shown in FIG. 2A. In embodiments, the P1 region of the Y adapter has the S1 sequence (SEQ ID NO:1) and the P2′ region of the Y adapter has the S2 sequence (SEQ ID NO:5), as described in Table 1 below. In embodiments, the P1 region of the Y adapter has the S4 sequence (SEQ ID NO:2) and the P2′ region of the Y adapter has the S5 sequence (SEQ ID NO:6), as described in Table 1 below. In embodiments, the P1 region of the Y adapter has the S7 sequence (SEQ ID NO:3) and the P2′ region of the Y adapter has the S8 sequence (SEQ ID NO:7), as described in Table 1 below. In embodiments, the P1 region of the Y adapter has the S9 sequence (SEQ ID NO:4) and the P2′ region of the Y adapter has the S10 sequence (SEQ ID NO:8), as described in Table 1 below.

TABLE 1
Sequences for the Y adapters. Note, the ′*′ is indicative of an
optional phosphorothioate linkage. Phosphorothioate linkages assist
in protecting the oligonucleotide against exonuclease degradation from
certain polymerases (e.g., phi29).
P1 regions of the Y adapter

| | |
|---|---|
| S1 (SEQ ID NO: 1) | /5Phos/ACAAAGGCAGCCACGCACTCCT |
| | TCCCTGAAGGCCGGAATC*T |
| S4 (SEQ ID NO: 2) | /5Phos/GCTGCCGCCACTAGCCATCTTAC |
| | TGCTGAGGACTCTTCGC*T |
| S7 (SEQ ID NO: 3) | /5Phos/ACAAAGGCAGCCACGCACTCCT |
| | TCCCT*G |
| S9 (SEQ ID NO: 4) | /5Phos/ACAAAGGCAGCCACGCACTCCT |
| | TCCCTG*T |

P2′ regions of the Y adapter

| | |
|---|---|
| S2 (SEQ ID NO: 5) | /5Phos/GATTCCGGCCTTGTGGTTGGTGA |
| | GGGTCATCTCGCTGGAG |
| S5 (SEQ ID NO: 6) | /5Phos/GCGAAGAGTCCTGGAGTGCCGC |
| | CAATGTATGCGAGGGTGA |
| S8 (SEQ ID NO: 7) | /5Phos/GTGGTTGGTGAGGGTCATCTCG |
| | CTGGAG |
| S10 (SEQ ID NO: 8) | /5Phos/AGTGGTTGGTGAGGGTCATCTC |
| | GCTGGAG |

As shown in FIG. 2D, the double-stranded region of the forked adapter may be blunt-ended (top), it may have a 3′ overhang (middle), or a 5′ overhang (bottom). The overhang may include a single nucleotide or more than one nucleotide. The 5′ end of the double-stranded part of the forked adapter is phosphorylated, i.e., the 5′ end of P2′. The presence of the 5′ phosphate group (referred to as 5′P in FIG. 2D) allows the adapter to ligate to the polynucleotide duplex. The 5′ end of P1 may be biotinylated or have a functional group at the end, thus enabling it to be immobilized on a surface (e.g., a planar solid support).

Alternatively, as depicted in FIG. 1B, the first adapter is a hairpin adapter (e.g., the hairpin adapter of FIG. 2B) and it is ligated to one end of a polynucleotide duplex.

The second adapter is a hairpin adapter (alternatively, it may be referred to as a stem-loop adapter, barbell, or hairpin loop adapter) and it is ligated to one end of a polynucleotide duplex, depicted as containing a P3 priming site in FIG. 1B and FIG. 2C. The hairpin adapter includes a double-stranded region which has a moderate Tₘ (e.g., 40-45° C.) so that it is stable during ligation, and includes at least 10 nucleotides. The hairpin adapter also includes a loop region which has a primer sequence and has an elevated Tₘ (e.g., 75° C.) relative to the double stranded region to enable stable binding of a complementary sequencing primer. The loop region or the stem region of the hairpin may further include a barcode or Unique Molecular Identifier (UMI) using degenerate sequences. The UMI consists of 3-5 degenerate nucleotides.

TABLE 2
Sequences for the hairpin adapter. Note, the ‘*’ is indicative of an optional
phosphorothioate linkage. Phosphorothioate linkages assist in protecting the
oligonucleotide against exonuclease degradation from certain polymerases (e.g., phi29).

| | |
|---|---|
| B1 | /5Phos/ GCGCGCG TTT TTT TT |
| (SEQ ID NO: 9) | GCTTGCGTCTCCTGCCAGCCATATCCGGTCTACGTGATCC TTT |
| | TTT TT CGCGCGC*T |
| B2 | /5Phos/GCGCGCGTTT TTT TTT TTT TT |
| (SEQ ID NO: 10) | GCTTGCGTCTCCTGCCAGCCATATCCGGTCTACGTGATCC TTT |
| | TTT TTT TTT TT CGCGCGC*T |
| B3 | /5Phos/GGATCACGTAGATTTTGCTTGCGTCTCCTGCCAGCCATA |
| (SEQ ID NO: 11) | TCCGGTTTTTCTACGTGATTCC*T |
| B4 | /5Phos/GCGAAGAGTCCT |
| (SEQ ID NO: 12) | GGAGTGCCGCCAATGTATGCGAGGGTGA |
| S4S5′_in loop_0Ts | GCTGCCGCCACTAGCCATCTTACTGCTG AGGACTCTTCGC*T |
| B5 | /5Phos/GCGAAGAGTCCT TTT TTT |
| (SEQ ID NO: 13) | GGAGTGCCGCCAATGTATGCGAGGGTGA |
| S4S5′_in loop_6Ts | GCTGCCGCCACTAGCCATCTTACTGCTG TTT TTT |
| | AGGACTCTTCGC*T |
| B6 | /5Phos/GCGAAGAGTCCT TTT TTT |
| (SEQ ID NO: 14) | GGAGTGCCGCCAATGTATGCGAGGGTGA TTT TTT T |
| S4S5′_in loop_6 + 8Ts | GCTGCCGCCACTAGCCATCTTACTGCTG TTT TTT |
| | AGGACTCTTCGC*T |
| B7 | /5Phos/GATTCCGGCCTT |
| (SEQ ID NO: 15) | GTGGTTGGTGAGGGTCATCTCGCTGGAGACAAAGGCAGCCAC |
| S1S2′_in loop_0Ts | GCACTCCTTCCCTGAAGGCCGGAATC*T |
| B8 | /5Phos/GATTCCGGCCTT TTT TTT |
| (SEQ ID NO: 16) | GTGGTTGGTGAGGGTCATCTCGCTGGAGACAAAGGCAGCCAC |
| S1S2′_in loop_6Ts | GCACTCCTTCCCTG TTTTTT AAGGCCGGAATC*T |
| B9 | /5Phos/GATTCCGGCCTT TTT TTT |
| (SEQ ID NO: 17) | GTGGTTGGTGAGGGTCATCTCGCTGGAGTTT TTT |
| S1S2′_in loop_6 + 7Ts | TACAAAGGCAGCCACGCACTCCTTCCCTG TTT TTT |
| | AAGGCCGGAATC*T |
| B10 | /5Phos/GGATCACGTAGATTTTGCTTGCGTCTCCTGCCAGCCATA |
| (SEQ ID NO: 18) | TCCGGTTTTTCTACGTGATCC*T |
| B11 | 5Phos/GG ATC ACG TAG ATT TTT TTT TTT TGC TTG CGT CTC |
| (SEQ ID NO: 19) | CTG CCA GCC ATA TCC GGT TTT TTT TTT TTT CTA CGT GAT |
| B10 + 12T | CC*T |
| B12 | 5Phos/GG ATC ACG TAG ATT TTT TTTTTT TTT TTT TTT TTT |
| (SEQ ID NO: 20) | TGC TTG CGT CTC CTG CCA GCC ATA TCC GGT TTT TTT TTT |
| B10 + 24T | TTT TTT TTT TTT TTT CTA CGT GAT CC*T |
| B13 | 5Phos/GG ATC ACG TAG ATT TTT TTT TTT TTT TTT TTT TTT |
| (SEQ ID NO: 21) | TTT TTT TTT TTT TTT TTG CTT GCG TCT CCT GCC AGC CAT |
| B10 + 40-10T | ATC CGG TTT TTT TTT TTC TAC GTG ATC C*T |
| B14 | /5Phos/GGA TCA CGT AGA TTT TAG ATC TGC TTG CGT CTC |
| (SEQ ID NO: 22) | CTG CCA GCC ATA TCC GGT TTT TCT ACG TGA TCC* T |
| B10 + clv | |
| B15 | /5Phos/GGA TCA CGT AGA TTTTTTTTTTTT AGA TCT GCT TGC |
| (SEQ ID NO: 23) | GTC TCC TGC CAG CCA TAT CCG GTTTTTTTTTTTTC TAC GTG |
| B11 + clv | ATC C*T |
| B16 | /5Phos/GTGGTTGGTGAG |
| (SEQ ID NO: 24) | GGTCATCTCGCTGGAG |
| S7S8′_in loop_0Ts | ACAAAGGCAGCCACGCACTCCTTCCCT*G |
| B17 | /5Phos/GTGGTTGGTGAG TTT TTT |
| (SEQ ID NO: 25) | GGTCATCTCGCTGGAG ACAAAGGCAGCCACG TTT TTT |
| S7S8′_in loop_6Ts | CACTCCTTCCCT*G |
| B18 | /5Phos/GTGGTTGGTGAG TTT TTT |
| (SEQ ID NO: 26) | GGTCATCTCGCTGGAG TTTTTTT ACAAAGGCAGCCACG TTT |
| S7S8′_in loop_6 + 7Ts | TTT CACTCCTTCCCT*G |
| B19 | /5Phos/AGTGGTTGGTGA |
| (SEQ ID NO: 27) | GGGTCATCTCGCTGGAG |
| S9S10′_in loop_0Ts | ACAAAGGCAGCCACGCACTCCTTCCCTG*T |
| B20 | /5Phos/AGTGGTTGGTGA TTT TTT |
| (SEQ ID NO: 28) | GGGTCATCTCGCTGGAG ACAAAGGCAGCCACGC TTT TTT |
| S9S10′_in loop_6Ts | ACTCCTTCCCTG*T |
| B21 | /5Phos/AGTGGTTGGTGA TTT TTT |
| (SEQ ID NO: 29) | GGTCATCTCGCTGGAG TTTTTTT ACAAAGGCAGCCACGC TTT |
| S9S10′_in loop_6+7Ts | TTT ACTCCTTCCCTG*T |

In embodiments, a hairpin adapter includes a sequence selected from SEQ ID NOs:9-29. In embodiments, the hairpin adapter has the B1 (SEQ ID NO:9) sequence described in Table 2. In embodiments, the hairpin adapter has the B2 (SEQ ID NO:10) sequence described in Table 2. In embodiments, the hairpin adapter has the B3 (SEQ ID NO:11) sequence described in Table 2. In embodiments, the hairpin adapter has the B4 (SEQ ID NO:12) sequence described in Table 2. In embodiments, the hairpin adapter has the B5 (SEQ ID NO:13) sequence described in Table 2. In embodiments, the hairpin adapter has the B6 (SEQ ID NO:14) sequence described in Table 2. In embodiments, the hairpin adapter has the B7 (SEQ ID NO:15) sequence described in Table 2. In embodiments, the hairpin adapter has the B8 (SEQ ID NO:16) sequence described in Table 2. In embodiments, the hairpin adapter has the B9 (SEQ ID NO:17) sequence described in Table 2. In embodiments, the hairpin adapter has the B10 (SEQ ID NO:18) sequence described in Table 2. In embodiments, the hairpin adapter has the B 11 (SEQ ID NO:19) sequence described in Table 2. In embodiments, the hairpin adapter has the B12 (SEQ ID NO:20) sequence described in Table 2. In embodiments, the hairpin adapter has the B13 (SEQ ID NO:21) sequence described in Table 2. In embodiments, the hairpin adapter has the B14 (SEQ ID NO:22) sequence described in Table 2. In embodiments, the hairpin adapter has the B15 (SEQ ID NO:23) sequence described in Table 2. In embodiments, the hairpin adapter has the B16 (SEQ ID NO:24) sequence described in Table 2. In embodiments, the hairpin adapter has the B17 (SEQ ID NO:25) sequence described in Table 2. In embodiments, the hairpin adapter has the B18 (SEQ ID NO:26) sequence described in Table 2. In embodiments, the hairpin adapter has the B19 (SEQ ID NO:27) sequence described in Table 2. In embodiments, the hairpin adapter has the B20 (SEQ ID NO:28) sequence described in Table 2. In embodiments, the hairpin adapter has the B21 (SEQ ID NO:29) sequence described in Table 2.

As shown in FIG. 2D, the double-stranded region of the hairpin adapter may be blunt-ended (top), it may have a 5′ overhang (middle), or a 3′ overhang (bottom). The overhang may include a single nucleotide or more than one nucleotide. The 5′ end of the double-stranded part of the hairpin adapter is phosphorylated. The presence of the 5′ phosphate group allows the adapter to ligate to the polynucleotide duplex.

The order of ligation events is not relevant, however for the purposes of discussion the terms ‘first’ and ‘second’ are used in reference to the sequence in which the adapter is ligated to the polynucleotide duplex. It is understood that the ligation of the Y adapter or the hairpin adapter may occur first, such that the resulting adapter-target-adapter constructs contain non-identical adapters.

Note, during this step it is possible to form adapter dimers (i.e., two adapters ligate together with no intervening template nucleic acid). There are several ways to reduce adapter dimer formation in the adapter ligation NGS library preparation described herein, including i) a stringent purification step (e.g., SPRI) after 3′ adapter ligation to remove non-ligated 3′ adapter molecules, prior to the second ligation of the 5′ adapter; ii) the use of A-tailed DNA and T-overhang adapters; iii) or utilizing alkaline phosphatase treatment after 3′ adapter ligation, before any SPRI cleanup, to remove 5′ phosphate group from the 3′ adapter to render any carryover 3′ adapter to be ligation incompatible and inert in the 5′ adapter ligation step.

### Methods

Fragmented DNA may be made blunt-ended by a number of methods known to those skilled in the art. In embodiments, the ends of the fragmented DNA are end repaired with T4 DNA polymerase and Klenow polymerase, a procedure well known to those skilled in the art, and then phosphorylated with a polynucleotide kinase enzyme. A single ‘A’ deoxynucleotide is then added to both 3′ ends of the DNA molecules using Taq polymerase enzyme, producing a one-base 3′ overhang that is complementary to the one-base T overhang on the double-stranded end of the Y adapter and hairpin adapter. For example, in the presence of a T4 DNA ligase, an A overhang is created on both strands at the 3′ hydroxyl end of a target duplex polynucleotide. For example, using Blunt/TA Ligase Master Mix (NEB #M0367) includes a T4 DNA ligase in a reaction buffer and ligation enhancers to ensure efficient A tailing. It is preferable to polish or use a filling reaction to ensure the ends of the target duplex polynucleotide are blunt before adding the A overhang. Examples of ends that need polishing or filling include inserts generated by shearing or sonication. A number of DNA polymerases will remove DNA overhangs and/or can be used to fill in missing bases if there is a 3′ hydroxyl available for priming. Polymerases for such reactions include, but are not limited to, a T4 DNA polymerase, PFU, and the Klenow Fragment of DNA polymerase I.

A ligation reaction between the Y adapter, the hairpin adapter, and the DNA fragments is then performed using a suitable ligase enzyme (e.g. T4 DNA ligase) which joins one hairpin adapter and one Y adapter to each DNA fragment, one at either end, to form adapter-target-adapter constructs that somewhat resemble a bobby pin hair fastener (see FIG. 1A). Alternatively, a ligation reaction between a first hairpin adapter (e.g., FIG. 2B), and a different second hairpin adapter (e.g., FIG. 2C), and the DNA fragments is then performed using a suitable ligase enzyme (e.g.. T4 DNA ligase) which joins the first hairpin adapter and the second hairpin adapter to each DNA fragment, one at either end, to form adapter-target-adapter constructs (see FIG. 1B).

The products of this reaction can be purified from leftover unligated adapters that by a number of means (e.g., NucleoMag NGS Clean-up and Size Select kit, Solid Phase Reversible Immobilization (SPRI) bead methods such as AMPureXP beads, PCRclean-dx kit, Axygen AxyPrep FragmentSelect-I Kit), including size-inclusion chromatography, preferably by electrophoresis through an agarose gel slab followed by excision of a portion of the agarose that contains the DNA greater in size that the size of the adapter.

### Linked Duplex Sequencing: Methylated/Unmethylated Cytosine Conversion

Once the adapter-target-adapter template nucleic acid construct has been formed, it may then be immobilized onto a solid-support. As shown in FIG. 5A, a Y-template-hairpin construct containing modified cytosine nucleobases (depicted as triangles) is hybridized to an immobilized P2 primer. In the presence of a polymerase, a first extension is performed, immobilizing a copy of the original template, wherein the copy has guanine nucleobases paired with the modified cytosine nucleobases (i.e., hemi-methylated double-stranded DNA). A methyltransferase is then introduced (e.g., a DNMT1 methyltransferase), along with a source of methyl groups (e.g., S-adenosyl-L-methionine) to generate methyl groups on the extended strand that match the methylation pattern of the original template strand. Methyltransferases are known to those of skill in the art and will become apparent based on the present disclosure. DNMT1 is the most abundant DNA methyltransferase in mammalian cells and is considered to be the key maintenance methyltransferase due to its ability to predominantly methylate hemimethylated CpG di-nucleotides in the mammalian genome. This enzyme is 7 to 100-fold more active on hemimethylated DNA as compared with the unmethylated substrate in vitro. By combining a first extension and DNMT1 incubation on genomic DNA, one can achieve the replication of genomic DNA methylation status on a solid-phase support. Furthermore, the methylation replication loops can be performed multiple times which results in up to 32-fold increase of starting DNA for bisulfite conversion or enzyme conversion such as APOBEC or other agent that converts cytosine to uracil. Additional useful methyltransferases include DNMT3a and DNMT3b which are mammalian methyl transferases. Additional useful methyltransferases include DRM2, MET1, and CMT3 which are plant methyl transferases. An additional useful methyltransferase include Dam, which is a bacterial methyl transferase. According to one aspect, it is to be understood that DNMT1 or other suitable methyltransferases are used with a methyl donor compound and may be used with or without cofactors known to those of skill in the art. DNMT1 works in vitro at 95% efficiency without a cofactor, however, DNMT1 may be used with a cofactor such as NP95(Uhrf1) as described in Bashtrykov Pl, Jankevicius G, Jurkowska R Z, Ragozin S, Jeltsch A. The UHRF1 protein stimulates the activity and specificity of the maintenance DNA methyltransferase DNMT1 by an allosteric mechanism.

An additional methyltransferase includes the DNA methyltransferase from the hyperthermophilic archaeon Aeropyrum pernix K1 (M.ApeKI). M.ApeKI has been established as a thermostable DNA (cytosine-5)-methyltransferase, which adds a methyl group to the second cytosine in 5′-GCWGC-3′ (Hayashi M et al. Microbiol. Spectr. 2021; 9(2): e0018621).

A methyltransferase (e.g., DNMT1) that transfers methyl groups specifically for hemimethylated DNA in any CpG context (i.e., not sequence specific) is advantageous for use in solid-phase amplification as described herein, as genomic fragments containing hemimethylated CpG DNA may be present with any combination of nucleotide bases on either end. The activity of DNMT1 toward unmethylated CG sites is at least 1000-fold reduced when compared with hemimethylated DNA. In the context of DNA carrying a defined methylation pattern, a 24-fold preference for hemimethylated target sites has been observed in vitro (Hermann A et al. J. Biol. Chem. 2004; 279(46):48350-9). In some experimental systems, DNMT1 has been reported to have de novo methyltransferase activity towards long terminal repeat (LTR) retrotransposons, specifically in the absence of DNMT3a and DNMT3b, where this mechanism may provide additional stability for long-term repression and epigenetic propagation throughout embryonic development (see, Haggerty C et al. Nat. Struct. Mol. Biol. 2021; 28(7): 594-603, which is incorporated herein by reference in its entirety).

Typically, methyltransferase reactions are performed at about 37° C., which is a lower temperature than what is normally used for extension by a strand-displacing polymerase (e.g., Bst LF). An advantage of performing the methyltransferase reaction as described supra on a template nucleic acid immobilized on a solid-phase support is that immobilized template can be retained while the buffer is exchanged, for example, to support a methylation reaction. As an example, a sequencing device including a flow cell can immobilize a template nucleic acid (e.g., anneal a template nucleic acid to an immobilized primer and, in the presence of a polymerase, extend the primer to create an immobilized complementary strand of the template nucleic acid), which typically occurs at an elevated temperature (e.g., about 50° C. to about 65° C.). Once the template has been immobilized, and while in double-stranded form, the temperature may then be reduced (e.g., to about 37° C.) and a methyltransferase reaction mixture is introduced (e.g., DNMT1 and SAM), and the methylation reaction is carried out. The methyltransferase step may be introduced, in embodiments, into a solid-phase amplification workflow, such that the original methylation status of the template nucleic acid is maintained in the amplicons.

FIG. 5B shows subsequent denaturation and washing away of the original template strand, allowing rehybridization of the immobilized, methyltransferase-treated strand into a Y-template-hairpin construct. A conversion technique may be applied as known in the art and described herein. For example, the enzymatic and chemical conversion method depicted in FIG. 4A may be applied, which converts the unmodified cytosine nucleobases to uracil analogs (depicted as squares). FIG. 5C shows an immobilized P1 primer annealed to the immobilized template polynucleotide. In the presence of a polymerase, an extension is performed. As in FIG. 5A, a methyltransferase is then introduced to generate methyl groups on the extended strand that match the methylation pattern of the original template strand. The process is then repeated to continue amplification of the template polynucleotide while retaining the original methylation information in the amplicons.

While bisulfite conversion is the current standard for performing DNA methylation analysis, it has several drawbacks. As discussed supra, bisulfite treatment is a harsh chemical reaction which can lead to DNA degradation, severely limiting its utility if sample DNA quantities are low, as is often the case with cfDNA. Additionally, the complete conversion of unmodified cytosine to thymine reduces sequencing complexity, potentially leading to poor sequencing quality, low mapping rates, and uneven genome coverage. A method for bisulfite-free direct detection of 5-methylcytosine (5mC) and 5-hydroxymethylcytosine (5hmC) has been described (Liu Y et al. Nat. Biotechnol. 2019; 37(4):424-429, which is incorporated herein by reference), which combines ten-eleven translocation (TET) oxidation of 5mC and 5hmC to 5-carboxylcytosine (5caC) with pyridine borane reduction of 5caC to dihydrouracil (DHU) (see, e.g., FIG. 3B and FIG. 4B). Subsequent PCR converts DHU to thymine, enabling a C-to-T transition of 5mC and 5hmC. Ibis TET-assisted pyridine borane sequencing (TAPS) method results in higher mapping rates and more even coverage than bisulfite conversion and may be applied to the methods described herein for linked duplex methylation profiling. Another bisulfite-free approach for methylation analysis is the NEBNext® Enzymatic Methyl-seq (EM-seq) product, which first protects 5mC and 5hmC from deamination by TET2 and an oxidation enhancer, followed by APOBEC deamination of unprotected cytosines to uracils (see, e.g., FIG. 4C).

Converted DNA can subsequently be analyzed by conventional molecular techniques, such as PCR amplification, sequencing, and detection including oligonucleotide hybridization. As described below, a variety of techniques are available for sequence-specific analysis (e.g., MSP) of the methylation status of one or more CpG dinucleotides in a particular region of interest. Methods provided herein are particularly useful for creating a reference complimentary copy of the pre-conversion sequence for each of a multitude of genomic fragments. Using these methods, the reference copy may be covalently linked to the converted template. By linking parent strands together using the constructs described herein (e.g., the hairpin adapter depicted in FIG. 2C), the sequence can be corrected prior to mapping using the second strand, increasing the fraction of properly mapped reads. Additionally, C to T mutations (SNVs) are distinguishable from converted bases as the “T” mutation will be confirmed by an “A” on the opposite strand enabling both detection of sequencing variants and methylation state in the same assay.

The DNA is fragmented, repaired, and adapters ligated as described supra. As a result of cytosine conversion, unmethylated cytosines in the template nucleic acid are converted to uracil residues, while methylated cytosines are unchanged (see, for example, FIG. 3A). In embodiments, the cytosine-converted construct may be amplified prior to hybridization to increase the amount of material available for cluster amplification, resulting in conversion of the uracil nucleotides (dUTP) to thymine nucleotides (dTTP). In embodiments where the adapter oligonucleotides include a sequence that will be used in later steps (i.e., for capture on a support or for binding of a sequencing primer), the adapter can be synthesized, for example, using a bisulfite-resistant cytosine analog such as 5-methyl dCTP (Me-C, or 5mC) in the positions where maintaining a cytosine at that position is important. Alternatively, a hairpin adapter could be ligated to one side of a linear template, with the hairpin adapter functioning as a primer to fill in the second strand of the template with dNTPs including Me-C. Following cytosine conversion, the second strand remains unconverted due to the incorporated Me-C bases and can serve as a reference for the original converted template strand.

Described herein are methods for amplifying DNA to produce amplicons having the methylation information or status of the original template DNA. The methods described herein provide for solid-phase amplification techniques that maintain the methylation status of the template nucleic acid, improving sequencing accuracy by ensuring that the methylated nucleobases are retained in the amplicons. Using these methods and/or compositions, the complexity of the target nucleic acids is preserved by keeping track of complementary strands after being subjected to, for example, bisulfite conversion of nucleic acids. In order to preserve complexity of the nucleic acid, embodiments of the present invention relate to a pairing of the cytosine-converted sequences of both strands of a double-stranded nucleic acid and using the sequence information from both strands to determine the sequence and/or methylation status of one or both strands prior to conversion.

### Duplex Sequencing: Methylated/Unmethylated Cytosine Conversion

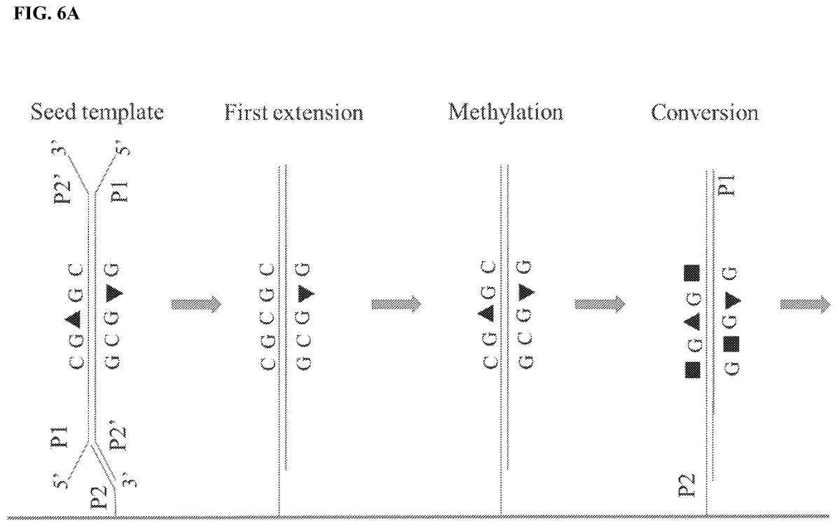

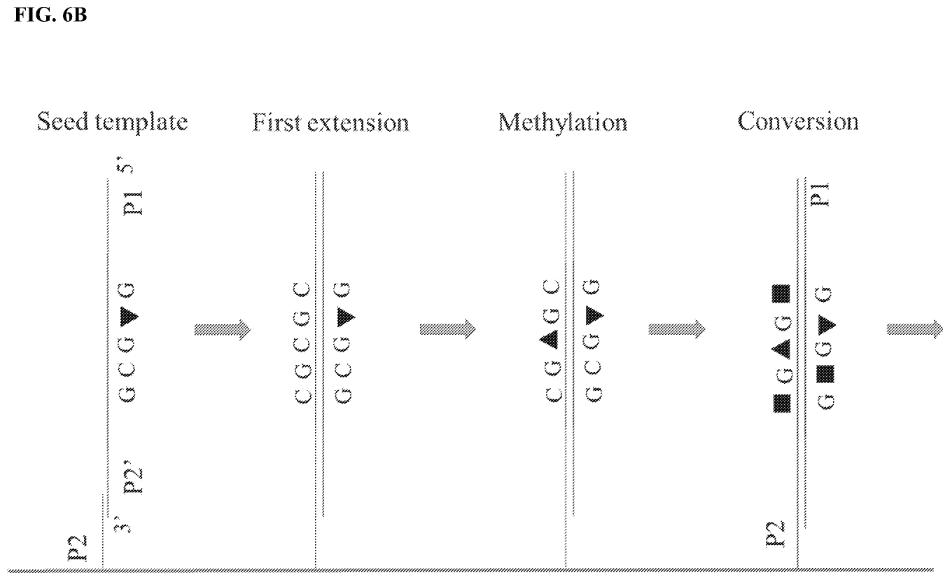

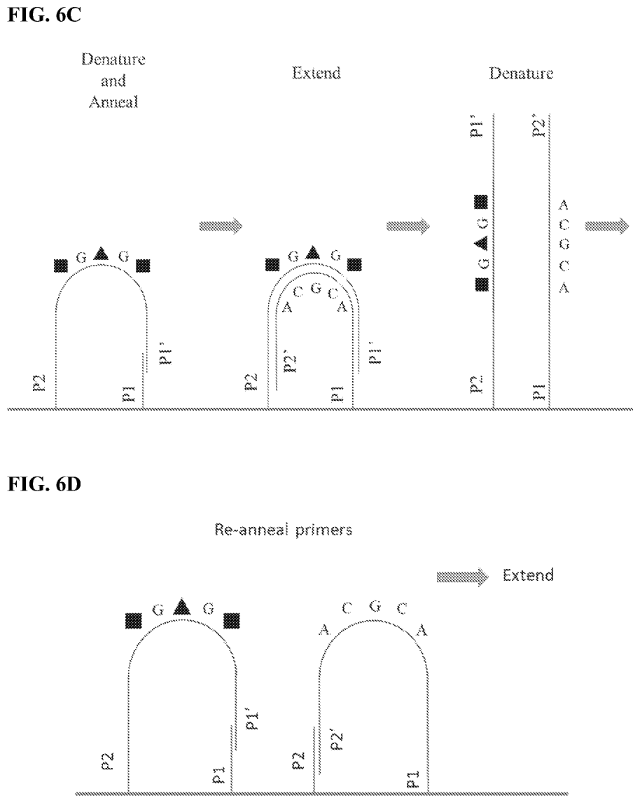

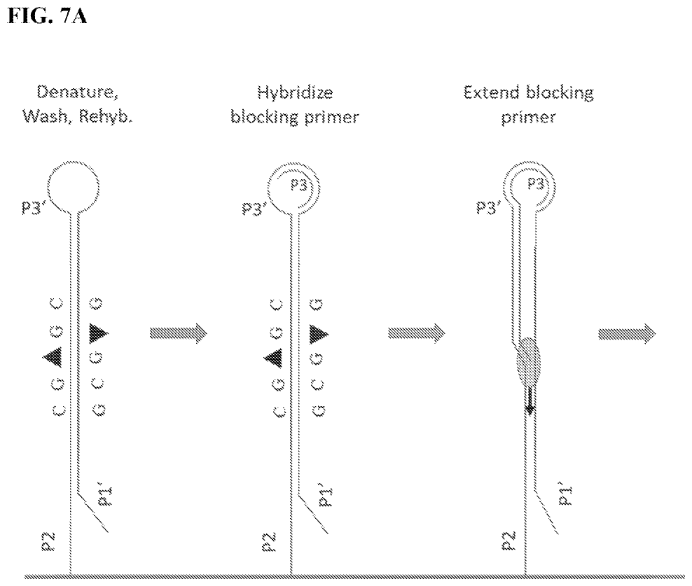

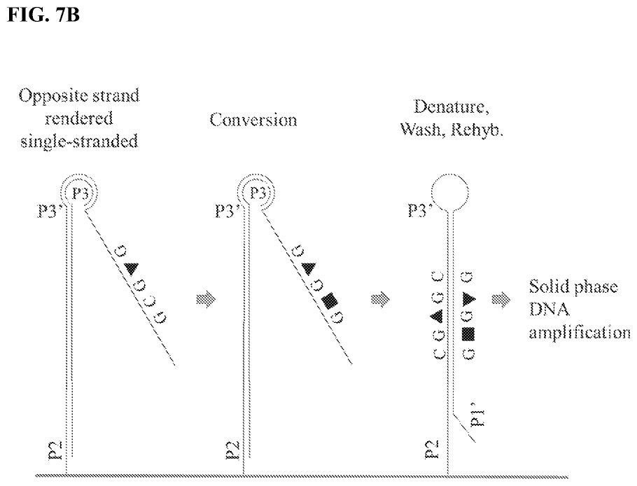

The methods described supra for preserving the methylation status of a Y-template-hairpin construct may also be applied to a non-linked duplex nucleic acid, for example, a non-linked double-stranded DNA template nucleic acid. In embodiments, the double-stranded DNA template nucleic acid includes a Y-shaped adapter on each end, as shown in FIG. 6A. The methods described herein are compatible with any adapter type that may be used on a double-stranded DNA template nucleic acid for subsequent immobilization on a solid support. As shown in FIG. 6A, a Y-template-Y construct containing modified cytosine nucleobases (depicted as triangles) is hybridized to an immobilized P2 primer. In the presence of a polymerase, a first extension is performed, immobilizing a copy of the original template, wherein the copy has guanine nucleobases paired with the modified cytosine nucleobases (i.e., hemi-methylated double-stranded DNA). A methyltransferase is then introduced (e.g., a DNMT1 methyltransferase), along with a source of methyl groups (e.g., S-adenosyl-L-methionine) to generate methyl groups on the extended strand that match the methylation pattern of the original template strand. A conversion technique may be applied as known in the art and described herein. For example, the enzymatic and chemical conversion method depicted in FIG. 4A may be applied, which converts the unmodified cytosine nucleobases to uracil analogs (depicted as squares).

Alternatively, the methods described herein may also be applied to a single-stranded DNA template nucleic acid, as shown in FIG. 6B. FIG. 6B shows a single-stranded DNA template containing modified cytosine nucleobases (depicted as triangles) hybridizing to an immobilized P2 primer and being processed as in FIG. 6A. In the presence of a polymerase, a first extension is performed, immobilizing a copy of the original template, wherein the copy has guanine nucleobases paired with the modified cytosine nucleobases (i.e., hemi-methylated double-stranded DNA). A methyltransferase is then introduced (e.g., a DNMT1 methyltransferase), along with a methyl donor compound (e.g., S-adenosyl-L-methionine) to generate methyl groups on the extended strand that match the methylation pattern of the original, double-stranded template nucleic acid from which the single-stranded DNA originated. A conversion technique may be applied as known in the art and described herein. For example, the enzymatic and chemical conversion method depicted in FIG. 4A may be applied, which converts the unmodified cytosine nucleobases to uracil analogs (depicted as squares).

The converted templates of FIGS. 6A and 6B may then be taken through an amplification process including a methyltransferase step as in FIG. 5C using immobilized P1 and P2 primers, thereby retaining the original methylation pattern in the template strands. Alternative, FIG. 6C shows an immobilized P1 primer annealed to the immobilized template polynucleotide. In the presence of a polymerase, an extension is performed. No methyltransferase step is performed following extension. The strands are then denatured and re-annealed to immobilized P1 and P2 primers, as shown in FIG. 6D. The process is then repeated to continue cluster amplification of the template polynucleotide.

Linked Duplex Sequencing: Methylated/Unmethylated Cytosine Conversion with Blocking Strand

Methods for cytosine conversion exhibit preferential activity towards single-stranded DNA, and typically require denaturation of a double-stranded template prior to performing a conversion reaction (e.g., chemical and/or enzymatic conversion). In a typical cytosine conversion protocol, a double-stranded template is melted into single-stranded polynucleotides, and each strand is subjected to the cytosine conversion method being performed. Described herein is a method that takes advantage of the bobby-pin fastener shape (i.e., a Y-shaped adapter on one end of a template and a hairpin adapter on a second end of the template) to generate a template nucleic acid with converted cytosines present on only one strand. In embodiments, the template nucleic acid has been immobilized onto a solid-support and treated with a methyltransferase to retain the original methylation status on both strands, as described herein. Ibis method provides an unmodified polynucleotide strand in the linked complementary strand of the template nucleic acid following the cytosine conversion protocol, providing an internal reference for modifications present on each template nucleic acid molecule that can subsequently be sequenced, simultaneously increasing sequencing depth and accurate detection of nucleobase modifications.

FIGS. 6A-6B illustrate an alternate embodiment of an amplification method for methylome analysis. FIG. 6A illustrates a template nucleic acid containing a first Y adapter, a double-stranded nucleic acid, and a hairpin adapter. The template nucleic acid has been immobilized and methylated as described in FIG. 5A. The double-stranded nucleic acid includes modified cytosine nucleobases, illustrated as triangles on both strands of the nucleic acid. In this embodiment, a primer anneals to the loop region of the hairpin and is extended by a strand-displacing polymerase (depicted as the grey ellipse) to generate a blocking strand. The blocking strand is hybridized to one of the two strands of the double-stranded nucleic acid, whereas the other strand is rendered single-stranded (FIG. 6B). Now that one of the two strands from the original dsDNA template is liberated, a conversion technique may be applied as known in the art and described herein. For example, the enzymatic and chemical conversion method depicted in FIG. 4A may be applied, which converts the modified cytosine nucleobases (depicted as triangles) to uracil analogs (depicted as squares) as shown in FIG. 6B. Once the blocking strand is removed, the template nucleic acid may reanneal, providing a template nucleic acid with asymmetric modifications (i.e., one strand contains modified cytosine nucleobases and the other strand contains converted cytosines (e.g., uracil analogs)). Solid phase DNA amplification may then be performed, for example, as depicted in FIG. 5C.

### Example 3. Linked Duplex Sequencing: Clustering and Use in Sequencing

### Linked Duplex Sequencing: Clustering Amplification

Once formed, the library of adapter-target-adapter templates prepared according to the methods described above can be used for solid-phase nucleic acid amplification. In some embodiments of the invention, the templates used for solid-phase nucleic acid amplification have been treated with bisulfite to convert any unmethylated cytosines to uracils using protocols known in the art. In other embodiments of the invention, the templates used for solid-phase nucleic acid amplification were subjected to TET oxidation of 5mC and 5hmC to 5caC with pyridine borane reduction of 5caC to DHU, as described supra. Other suitable methods known in the art for detecting 5mC and 5hmC, including other enzymatic methods such as EM-seq and those described herein may also be applied to templates for subsequent solid-phase nucleic acid amplification.

Thus, in another aspect is provided a method of nucleic acid amplification of template polynucleotide molecules which includes preparing a library of template polynucleotide molecules (e.g., adapter-target-adapter templates) and performing an amplification reaction (e.g., a solid-phase nucleic acid amplification reaction) wherein the template polynucleotide molecules are amplified while retaining the methylation status of the original template nucleic acids. As shown in FIG. 5C, the complimentary copy of the cytosine converted template polynucleotide is annealed to a P1 primer that is immobilized on the solid substrate, which in the presence of a DNA polymerase (the polymerase is not shown in FIG. 5C) extends P1 primer. The products of the extension reaction (i.e., the P1-template-P3-template-P2′ hybridized to an immobilized P2, and P1′-template-P3′-template-P2 hybridized to P1) may then be subjected to a methyltransferase reaction (e.g., with DNMT1 and SAM), followed by standard denaturing conditions in order to separate the extension products from strands of the adapter-target constructs. The adapter-target-adapter constructs may then anneal to a complementary immobilized primer and may be extended in the presence of a polymerase. These steps, depicted in FIG. 5C, may be repeated one or more times, through rounds of primer annealing, extension, methylation, and denaturation, in order to form multiple copies of the same extension products containing adapter-target-adapter constructs, or the complements thereof. The A/C and U/G mismatches are carried forward through each round of amplification (not shown for clarity on far-right panel of FIG. 5C). Note, this bridging amplification is typically more efficient than amplifying linear strands, because the adapter-target-adapter products self-fold, thus leaving the primer site accessible.

Alternatively, as shown in FIG. 6C for a non-linked strand template nucleic acid, the immobilized cytosine converted template polynucleotide from FIG. 6A or FIG. 6B is annealed to a P1 primer that is immobilized on the solid substrate, which in the presence of a DNA polymerase (the polymerase is not shown in FIG. 6C) extends P1 primer. The products of the extension reaction (i.e., the immobilized P1-template-P2′ hybridized to an immobilized P1′-template-P2) are not subjected to a methyltransferase reaction as in FIG. 5C but are followed by standard denaturing conditions in order to separate the extension products from strands of the adapter-target constructs. The adapter-target-adapter constructs may then anneal to a complementary immobilized primer, as shown in FIG. 6D, and may be extended in the presence of a polymerase. These steps, depicted in FIG. 6C, may be repeated one or more times, through rounds of primer annealing, extension, and denaturation, in order to form multiple copies of the same extension products containing adapter-target-adapter constructs, or the complements thereof. The A/C and U/G mismatches are carried forward through each round of amplification (not shown for clarity on far-right panel of FIG. 5C).

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition. In embodiments, detection of a methylated cytosine is determined by the presence of a G-T or T-C mismatch following sequencing of the amplified converted template nucleic acid. The type of mismatch that is detected is dependent on the method(s) used for cytosine conversion, for example, whether bisulfite conversion or TAPS is used. Using the methods described herein, SNPs are distinguishable from converted G-T or T-C base pairs.

The term “solid-phase amplification” as used herein refers to any nucleic acid amplification reaction carried out on or in association with a solid support such that all or a portion of the amplified products are immobilized on the solid support as they are formed. The term encompasses solid-phase polymerase chain reaction (solid-phase PCR), which is a reaction analogous to standard solution phase PCR, except that both of the forward and reverse amplification primers (referred to herein as P1 and P2) are immobilized on the solid support. In practice, there will be a “plurality” of identical forward primers and/or a “plurality” of identical reverse primers immobilized on the solid support, since the PCR process requires an excess of primers to sustain amplification.

In embodiments, amplification primers for solid-phase amplification are preferably immobilized by covalent attachment to the solid support at or near the 5′ end of the primer, leaving the template-specific portion of the primer free for annealing to the cognate template and the 3′ hydroxyl group free for primer extension. Any suitable covalent attachment means known in the art may be used for this purpose. The primer itself may include a moiety, which may be a non-nucleotide chemical modification, to facilitate attachment. In embodiments, the primer may include a sulfur-containing nucleophile (e.g., phosphorothioate or thiophosphate) at the 5′ end.

In embodiments, the adapter-target-adapter templates prepared according to the methods described above can be used to prepare clustered arrays of nucleic acid colonies by solid-phase PCR amplification. The terms “cluster” and “colony” are used interchangeably herein to refer to a discrete site on a solid support comprised of a plurality of immobilized nucleic acid strands and a plurality of immobilized complementary nucleic acid strands. The term “clustered array” refers to an array formed from such clusters or colonies. In this context the term “array” is not to be understood as requiring an ordered arrangement of clusters.

### Linked Duplex Sequencing: Use in Sequencing

In another aspect is provided methods of sequencing amplified nucleic acids, optionally generated by the amplification methods described herein. The method includes optionally removing all or a portion of one immobilized strand in a “bridged” double-stranded nucleic acid structure (i.e. linearizing) and sequencing.

The products of solid-phase amplification reactions described herein wherein both P1 and P2 primers are covalently immobilized on the solid surface and may be referred to as “bridged structures” formed by annealing of pairs of immobilized polynucleotide strands and immobilized complementary strands, both strands being attached to the solid support at the 5′ end.

Arrays comprised of such bridged structures provide inefficient templates for nucleic acid sequencing, since hybridization of a conventional sequencing primer to one of the immobilized strands is not preferred compared to annealing of this strand to its immobilized complementary strand under standard conditions for hybridization. In order to provide more suitable templates for nucleic acid sequencing it is preferred to remove substantially all or at least a portion of one of the immobilized strands in the “bridged” structure in order to generate a template which is at least partially single-stranded. The portion of the template which is single-stranded will thus be available for hybridization to a sequencing primer. The process of removing all or a portion of one immobilized strand in a “bridged” double-stranded nucleic acid structure may be referred to herein as “linearization”. Bridged template structures may be linearized by cleavage of one or both strands with a restriction endonuclease or by cleavage of one strand with a nicking endonuclease. Other methods of cleavage can be used as an alternative to restriction enzymes or nicking enzymes, including chemical cleavage (e.g. cleavage of a diol linkage with periodate), cleavage of abasic sites by cleavage with endonuclease, or by exposure to heat or alkali, cleavage of ribonucleotides incorporated into amplification products otherwise comprised of deoxyribonucleotides, photochemical cleavage or cleavage of a peptide linker. Alternatively, the primers may be attached to the solid surface with a cleavable linker, such that upon exposure to a cleaving agent, all or a portion of the primer is removed from the surface.

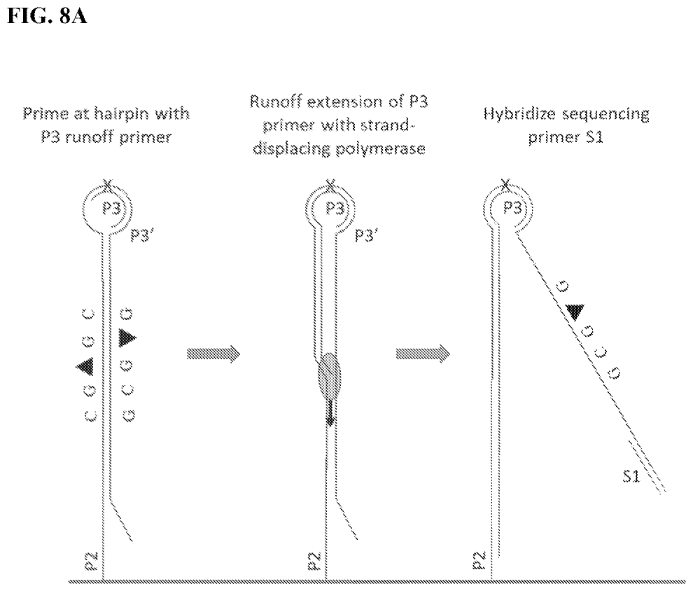

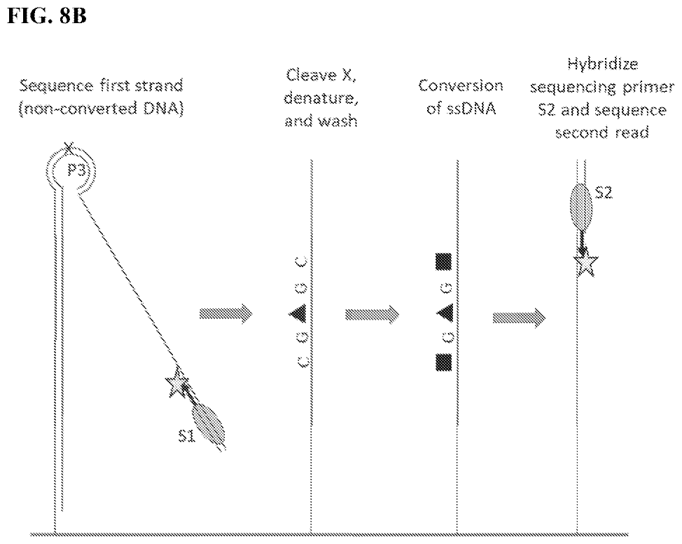

Sequencing reactions: The initiation point for the first sequencing reaction is provided as illustrated in FIG. 8A, wherein a nucleic acid template containing a first Y adapter, a double-stranded nucleic acid, and a hairpin adapter, wherein the hairpin adapter contains a cleavable site (i.e., the cleavable site is indicated as ‘X’). The template nucleic acid has been immobilized and methylated as described in FIG. 5A. The double-stranded nucleic acid includes modified cytosine nucleobases, illustrated as triangles on both strands of the nucleic acid. In this embodiment, a runoff primer anneals to the loop region of the hairpin and is extended by a strand-displacing polymerase (depicted as the grey ellipse) to generate an invasion strand. The invasion strand is hybridized to one of the two strands of the double-stranded nucleic acid, whereas the other strand is rendered single-stranded. A sequencing primer S1 is then hybridized to the single-stranded end. FIG. 8B shows sequencing with detectable nucleotides (indicated by the star) from the S1 primer, followed by cleavage at the cleavable site, denaturation, and washing to leave behind the immobilized single-stranded strand. A conversion technique may be applied as known in the art and described herein. For example, the enzymatic and chemical conversion method depicted in FIG. 4A may be applied, which converts the modified cytosine nucleobases (depicted as triangles) to uracil analogs (depicted as squares).

Next, a second sequencing reaction is initiated by annealing a sequencing primer S2 complementary to a region in the hairpin (e.g., P3), and in the presence of a polymerase, nucleotides (e.g., labeled nucleotides) are incorporated and detected such that the identity of the incorporated nucleotides allows for the identification of the second template strand. Thus, the second sequencing reaction may include hybridizing a sequencing primer to a region of a linearized amplification product, sequentially incorporating one or more nucleotides into a polynucleotide strand complementary to the region of amplified template strand to be sequenced, identifying the base present in one or more of the incorporated nucleotide(s) and thereby determining the sequence of a region of the template strand.

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′—OH group available to direct further sequence extension and therefore the polymerase cannot add further nucleotides. Once the identity of the base incorporated into the growing chain has been determined, the 3′ reversible terminator may be removed to allow addition of the next successive nucleotide. Such reactions can be done in a single experiment if each of the modified nucleotides has a different label attached thereto, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. For example, the detectable label can be a paramagnetic spin label such as nitroxide and detected by electron paramagnetic resonance and related techniques. Exemplary spin labels and techniques for their detection are described in Hubbell et al. Trends Biochem Sci. 27:288-95 (2002), which is incorporated herein by reference in its entirety. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected by a detection apparatus (e.g., by a CCD camera or other suitable detection means).

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

### Example 4. Enzymatic-Based Approaches for Methylation Detection

As described supra, a common method of determining the methylation level and/or pattern of DNA is bisulfite conversion, a process in which genomic DNA is denatured (i.e., rendered single-stranded) and treated with sodium bisulfite, leading to deamination of unmethylated cytosine nucleobases into uracil nucleobases, while methylated cytosine nucleobases (e.g., 5-methylcytosine and 5-hydroxymethylcytosine) remain unchanged. Thus, thymine nucleobases detected in bisulfite sequencing correspond to either thymine nucleobases or unmethylated cytosine nucleobases in the original DNA, and alignment with the original template sequence easily differentiates between them. An alternative method for bisulfite-free detection of modified cytosine nucleobases (e.g., 5-methylcytosine (5mC) and 5-hydroxymethylcytosine (5hmC)) has been described (Liu Y et al. Nat. Biotechnol. 2019; 37(4): 424-429, which is incorporated herein by reference), which utilizes an enzymatic approach, and combines the ten-eleven translocation (TET) oxidation of 5mC and 5hmC to 5-carboxylcytosine (5caC) with pyridine borane reduction of 5caC to dihydrouracil (DHU). Subsequent PCR converts DHU to thymine, enabling a C-to-T transition of 5mC and 5hmC. This TET-assisted pyridine borane sequencing (TAPS) method results in higher mapping rates and more even coverage than bisulfite conversion and may be applied to the methods described herein for linked duplex methylation profiling.

Another bisulfite-free approach for methylation analysis is Enzymatic Methyl-seq (EM-Seq, described in Vaisvila R et al. bioRxiv. 2019; 12.20.884692, which is incorporated herein by reference), which uses TET2 to catalyze the oxidization of 5mC to 5hmC, 5-formulcytosine (5fC), and 5caC. T4-phage β glucosyltransferase (T4-βGT) then catalyzes the glucosylation of the formed 5hmC to 5-(β-glucosyloxymethyl)cytosine (5gmC). Combining T4-βGT with TET2 effectively protects both 5mC and 5hmC, but not cytosines, from subsequent deamination by APOBEC3A to uracils. This enzymatic manipulation of cytosine, 5mC and 5hmC enables discrimination of 5mC/5hmC from cytosine in high-throughput sequence data. 5mC and 5hmC are sequenced as cytosines, whereas unmodified cytosines are sequenced as thymines.

Two additional bisulfite-free methods for detecting cytosine modifications have been recently developed, TAPS with β-glucosyltransferase (TAPSβ) and chemical-assisted pyridine borane sequencing (CAPS), allowing 5mC-specific and 5hmC-specific sequencing, respectively (for additional details, see Liu Y et al. Nature Comm. 2021; 12: 618, which is incorporated herein by reference). The TAPSβ method uses βGT for selective labeling of 5hmC with glucose that enables 5hmC pulldown and protection from TET oxidation or APOBEC deamination. Following 5hmC blocking, TET oxidation and borane (e.g., pyridine borane) reduction is performed on 5mC as described in the TAPS method supra. Thereafter, 5mC are sequenced as thymines, whereas 5hmC are sequenced as cytosines. In the CAPS approach, chemical oxidation of 5hmC to 5fC is performed, which can also be converted to DHU by borane reduction. Thereafter, 5hmC are sequenced as thymines. In CAPS, the oxidation step is performed with a chemical oxidant, for example, potassium perruthenate (KRuO₄) or potassium ruthenate (K₂RuO₄). Both of these oxidants only work on single-stranded DNA. Borane reduction may be performed, for example, with 2-methylpyridine borane (pic-borane) or pyridine borane. In some embodiments, both TAPSβ and CAPS may be applied for detecting cytosine modifications to discriminate between 5mC and 5hmC modifications.

The methods described supra for enzymatic modified cytosine conversion may be applied to the sequencing workflow described in Example 2. For example, a template nucleic acid containing one or more cytosine nucleobases, wherein one or more of the cytosine nucleobases include modified cytosine nucleobases is ligated to a first adapter and a second adapter, forming a linked paired strand nucleic acid template. In some embodiments, after generating a linked paired strand template nucleic acid, the one or more cytosine nucleobases are converted to a uracil nucleobase or a uracil nucleobase analog. In some embodiments, converting the one or more cytosine nucleobases includes the TET-assisted pyridine borane sequencing (TAPS) method. In embodiments, converting the one or more cytosine nucleobases includes the Enzymatic Methyl-seq (EM-Seq) method. In some embodiments, converting the one or more cytosine nucleobases includes performing the TAPS with β-glucosyltransferase (TAPSβ). In some embodiments, converting the one or more cytosine nucleobases includes performing the chemical-assisted pyridine borane sequencing (CAPS) method.

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition. In embodiments, detection of a methylated cytosine is determined by the presence of a G-T mismatch following sequencing of the amplified converted template nucleic acid. Using the methods described herein, SNPs are distinguishable from converted G-T base pairs.

### P-EMBODIMENTS

The present disclosure provides the following illustrative embodiments.

Embodiment P1. A method of generating a methylated complement template polynucleotide comprising: (a) annealing a methylated template polynucleotide to a first immobilized primer on a solid support at a first temperature, wherein the first immobilized primer is complementary to a sequence of the methylated template polynucleotide; (b) extending the first primer with a polymerase to generate a non-methylated complement template polynucleotide; and (c) contacting the non-methylated complement template polynucleotide with a methyltransferase reagent to generate a methylated complement template polynucleotide hybridized to said methylated template polynucleotide, wherein said methylated complement template polynucleotide comprises one or more methylated cytosine nucleobases and one or more non-methylated cytosine nucleobases.

Embodiment P2. The method of Embodiment P1, further comprising denaturing the methylated complement template polynucleotide from said methylated template polynucleotide at a second temperature, wherein the second temperature is higher than the first temperature.

Embodiment P3. The method of Embodiment P1 or Embodiment P2, further comprising contacting the methylated complement template polynucleotide with a chemical denaturant.

Embodiment P4. The method of Embodiment P1, further comprising contacting said methylated complement template polynucleotide with a conversion agent thereby converting said one or more non-methylated cytosine nucleobases to one or more uracil nucleobases and generating a uracil-containing strand.

Embodiment P5. The method of Embodiment P1, further comprising contacting said methylated complement template polynucleotide with a conversion agent thereby converting said one or more methylated cytosine nucleobases to one or more uracil or uracil analog nucleobases and generating a uracil-containing strand.

Embodiment P6. The method of Embodiment P4, further comprising contacting said methylated complement template polynucleotide with a second conversion agent thereby converting said one or more methylated cytosine nucleobases to one or more 5-carboxylcytosine (5caC) nucleobases.

Embodiment P7. The method of Embodiment P4 or Embodiment P5, comprising annealing a primer to the uracil-containing strand and extending with a polymerase to generate an amplification product.

Embodiment P8. The method of Embodiment P3, further comprising (d) removing the denaturant and annealing the methylated complement template polynucleotide to a second immobilized primer on said solid support at the first temperature, wherein the second immobilized primer is complementary to a sequence of the methylated complement template polynucleotide; and extending the second immobilized primer with the polymerase to generate a complement of the methylated complement template polynucleotide.

Embodiment P9. The method of Embodiment P8, further comprising (e) contacting the complement of the methylated complement template polynucleotide with a methyltransferase reagent to generate a methylated template polynucleotide hybridized to said methylated complement template polynucleotide, wherein said methylated template polynucleotide comprises one or more methylated cytosine nucleobases, and contacting the methylated complement template polynucleotide with a conversion agent to convert the one or more non-methylated cytosine nucleobases to uracil nucleobases, thereby generating a uracil-containing strand comprising one or more uracil nucleobases.

Embodiment P10. The method of Embodiment P9, further comprising repeating steps (a) to (e), thereby amplifying the template polynucleotide.

Embodiment P11. The method of Embodiment P1 or Embodiment P2, wherein extending the first primer occurs at the first temperature.

Embodiment P12. The method of Embodiment P2, wherein extending the first primer occurs at the second temperature.

Embodiment P13. The method of any one of Embodiment P2 to Embodiment P12, further comprising contacting the methylated complement template polynucleotide with a denaturant at a temperature between the second temperature and the first temperature.

Embodiment P14. The method of any one of Embodiment P1 to Embodiment P13, wherein the first temperature is about 25° C. to about 45° C., or about 40° C. to about 45° C.

Embodiment P15. The method of any one of Embodiment P1 to Embodiment P13, wherein the first temperature is a temperature between about 25° C. to about 45° C.

Embodiment P16. The method of any one of Embodiment P1 to Embodiment P14, wherein the second temperature is about 45° C. to about 70° C., or about 55° C. to about 62° C.

Embodiment P17. The method of any one of Embodiment P1 to Embodiment P14, wherein the second temperature is a temperature between about 45° C. to about 70° C.

Embodiment P18. The method of any one of Embodiment P1 to Embodiment P17, wherein the first temperature increases to the second temperature at a fixed rate.

Embodiment P19. The method of any one of Embodiment P1 to Embodiment P18, further comprising prior to step a) contacting the solid support with a sample comprising a methylated template polynucleotide.

Embodiment P20. The method of any one of claims 1 to 19, wherein the methyltransferase reagent is DNMT1.

Embodiment P21. The method of any one of Embodiment P3 to Embodiment P20, wherein the chemical denaturant comprises formamide, ethylene glycol, sodium hydroxide, or a mixture thereof.

Embodiment P22. The method of any one of Embodiment P7 to Embodiment P21, further comprising contacting the amplification product with a chemical denaturant thereby separating the first strand and the amplification product; annealing a second immobilized primer to the second strand; and repeating step (c).

Embodiment P23. The method of any one of Embodiment P7 to Embodiment P22, further comprising contacting the amplification product with a methyltransferase reagent to generate a methylated amplification product.

Embodiment P24. The method of any one of Embodiment P1 to Embodiment P23, wherein the methylated template polynucleotide comprises one or more 5-methylcytosine (5mC) or 5-hydroxymethyl cytosine (5hmC) nucleobases.

Embodiment P25. The method of any one of Embodiment P1 to Embodiment P24, further comprising sequencing the methylated complement template polynucleotide.

Embodiment P26. A polynucleotide comprising a first nucleic acid sequence comprising one or more methylated cytosine nucleobases hybridized to a second nucleic acid sequence comprising one or more uracil nucleobases, wherein the polynucleotide comprises one or more cytosine mismatches.

### Additional Embodiments

The present disclosure provides the following additional illustrative embodiments.

Embodiment 1. A method of generating an immobilized methylated complement template polynucleotide, said method comprising: i) hybridizing a methylated template polynucleotide to a first immobilized primer at a first temperature, wherein said first immobilized primer is attached to a solid support; ii) extending the first immobilized primer with a polymerase to generate an immobilized non-methylated complement template polynucleotide hybridized to said methylated template polynucleotide; and iii) contacting the immobilized non-methylated complement template polynucleotide with a DNA methyltransferase reagent to generate an immobilized methylated complement template polynucleotide, wherein said methylated complement template polynucleotide comprises one or more methylated cytosine nucleobases and one or more non-methylated cytosine nucleobases.

Embodiment 2. The method of Embodiment 1, further comprising iv) denaturing the immobilized methylated complement template polynucleotide from said methylated template polynucleotide at a second temperature, wherein the second temperature is higher than the first temperature.

Embodiment 3. The method of Embodiment 2, further comprising v) repeating steps i)-iv), thereby generating a plurality of immobilized methylated polynucleotides.

Embodiment 4. The method of any one of Embodiments 2 to 3, wherein contacting the immobilized non-methylated complement template polynucleotide with the DNA methyltransferase reagent occurs at a third temperature, wherein said third temperature is lower than said first temperature.

Embodiment 5. The method of any one of Embodiments 2 to 4, wherein step iv) comprises contacting the immobilized methylated complement template polynucleotide with a chemical denaturant.

Embodiment 6. The method of any one of Embodiments 2 to 5, further comprising contacting said immobilized methylated complement template polynucleotide with a conversion agent thereby converting said one or more non-methylated cytosine nucleobases to one or more uracil nucleobases and generating an immobilized uracil-containing polynucleotide.

Embodiment 7. The method of any one of Embodiments 2 to 5, further comprising contacting said immobilized methylated complement template polynucleotide with a conversion agent thereby converting said one or more methylated cytosine nucleobases to one or more uracil or uracil analog nucleobases and generating an immobilized uracil-containing polynucleotide.

Embodiment 8. The method of Embodiment 6 or 7, further comprising contacting said immobilized methylated complement template polynucleotide with a second conversion agent thereby converting said one or more methylated cytosine nucleobases to one or more 5-carboxylcytosine (5caC) nucleobases.

Embodiment 9. The method of any one of Embodiments 6 to 8, comprising annealing a primer to the immobilized uracil-containing polynucleotide and extending the primer hybridized to the immobilized uracil-containing polynucleotide with a polymerase to generate an amplification product.

Embodiment 10. The method of any one of Embodiments 5 to 9, further comprising vi) removing the chemical denaturant and hybridizing the immobilized methylated complement template polynucleotide to a second immobilized primer at the first temperature, wherein said second immobilized primer is attached to the solid support; and extending said second immobilized primer with a polymerase to generate an immobilized complement of the immobilized methylated complement template polynucleotide hybridized to the immobilized methylated complement template polynucleotide.

Embodiment 11. The method of Embodiment 10, further comprising applying oxygen to the solid support prior to removing the chemical denaturant.

Embodiment 12. The method of Embodiment 10, further comprising applying air to the solid support prior to removing the chemical denaturant.

Embodiment 13. The method of any one of Embodiments 10 to 12, further comprising vii) contacting the immobilized complement of the methylated complement template polynucleotide with a DNA methyltransferase reagent to generate an immobilized methylated template polynucleotide hybridized to said immobilized methylated complement template polynucleotide, wherein said immobilized methylated template polynucleotide comprises one or more methylated cytosine nucleobases and one or more non-methylated cytosine nucleobases, and contacting the immobilized methylated complement template polynucleotide with a conversion agent to convert the one or more non-methylated cytosine nucleobases to uracil nucleobases, thereby generating an immobilized uracil-containing polynucleotide comprising one or more uracil nucleobases.

Embodiment 14. The method of Embodiment 13, further comprising repeating steps (i) to (vii), thereby amplifying the template polynucleotide.

Embodiment 15. The method of Embodiment 14, wherein amplifying the template polynucleotide comprises bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of said methods.

Embodiment 16. The method of any one of Embodiments 1 to 15, wherein extending the first primer occurs at the first temperature.

Embodiment 17. The method of any one of Embodiments 2 to 16, wherein extending the first primer occurs at the second temperature.

Embodiment 18. The method of any one of Embodiments 1 to 17, wherein the first temperature is about 40° C. to about 45° C.

Embodiment 19. The method of any one of Embodiments 2 to 18, wherein the second temperature is about 55° C. to about 65° C.

Embodiment 20. The method of any one of Embodiments 4 to 19, wherein the third temperature is about 30° C. to about 40° C.

Embodiment 21. The method of any one of Embodiments 2 to 20, wherein the first temperature and the second temperature differ by about 5° C. to about 15° C.

Embodiment 22. The method of any one of Embodiments 2 to 20, wherein the first temperature and the second temperature differ by no greater than 20° C.

Embodiment 23. The method of any one of Embodiments 2 to 22, wherein the first temperature increases to the second temperature at a controlled rate.

Embodiment 24. The method of any one of Embodiments 1 to 23, further comprising prior to step i) contacting the solid support with a sample comprising a methylated template polynucleotide.

Embodiment 25. The method of any one of Embodiments 1 to 24, wherein the DNA methyltransferase reagent comprises DNMT1.

Embodiment 26. The method of any one of Embodiments 3 to 25, wherein the chemical denaturant comprises formamide, ethylene glycol, sodium hydroxide, or a mixture thereof.

Embodiment 27. The method of Embodiment 26, wherein the chemical denaturant is formamide.

Embodiment 28. The method of any one of Embodiments 9 to 27, further comprising contacting the amplification product with a chemical denaturant thereby separating the first polynucleotide and the amplification product; annealing a second immobilized primer to the second polynucleotide; and repeating step (iii).

Embodiment 29. The method of any one of Embodiments 7 to 28, further comprising contacting the amplification product with a DNA methyltransferase reagent to generate a methylated amplification product.

Embodiment 30. The method of any one of Embodiments 1 to 29, wherein the methylated template polynucleotide comprises one or more 5-methylcytosine (5mC) or 5-hydroxymethyl cytosine (5hmC) nucleobases.

Embodiment 31. The method of any one of Embodiments 1 to 30, further comprising sequencing the immobilized methylated complement template polynucleotide.

Embodiment 32. The method of Embodiment 31, wherein sequencing comprises sequencing-by-synthesis, sequencing-by-binding, sequencing-by-ligation, or pyrosequencing.

Embodiment 33. The method of Embodiment 31, wherein sequencing comprises incorporating one or more nucleotides into a sequencing primer hybridized to the immobilized methylated complement template polynucleotide to generate an extension strand; and detecting the one or more incorporated nucleotides so as to identify each incorporated nucleotide in said extension strand, thereby sequencing the immobilized methylated complement template polynucleotide.

Embodiment 34. The method of any one of Embodiments 1 to 33, wherein the solid support is a bead.

Embodiment 35. The method of any one of Embodiments 1 to 33, wherein the solid support is a multiwell container comprising a plurality of wells, wherein each well comprises said first immobilized primer.

Embodiment 36. An immobilized polynucleotide comprising a first nucleic acid sequence comprising one or more methylated cytosine nucleobases hybridized to a second nucleic acid sequence comprising one or more uracil nucleobases, wherein the polynucleotide comprises one or more cytosine mismatches, and wherein the immobilized polynucleotide is attached to a solid support.

1. A method of generating an immobilized methylated complement template polynucleotide, said method comprising:
i) hybridizing a methylated template polynucleotide to a first immobilized primer at a first temperature, wherein said first immobilized primer is attached to a solid support;
ii) extending the first immobilized primer with a polymerase to generate an immobilized non-methylated complement template polynucleotide hybridized to said methylated template polynucleotide; and
iii) contacting the immobilized non-methylated complement template polynucleotide with a DNA methyltransferase reagent to generate an immobilized methylated complement template polynucleotide, wherein said methylated complement template polynucleotide comprises one or more methylated cytosine nucleobases and one or more non-methylated cytosine nucleobases.

2. The method of claim 1, further comprising iv) denaturing the immobilized methylated complement template polynucleotide from said methylated template polynucleotide at a second temperature, wherein the second temperature is higher than the first temperature.

3. The method of claim 2, further comprising v) repeating steps i)-iv), thereby generating a plurality of immobilized methylated polynucleotides.

4. The method of claim 2, wherein contacting the immobilized non-methylated complement template polynucleotide with the DNA methyltransferase reagent occurs at a third temperature, wherein said third temperature is lower than said first temperature.

5. The method of claim 2, wherein step iv) comprises contacting the immobilized methylated complement template polynucleotide with a chemical denaturant.

6. The method of claim 2, further comprising contacting said immobilized methylated complement template polynucleotide with a conversion agent thereby converting said one or more non-methylated cytosine nucleobases to one or more uracil nucleobases and generating an immobilized uracil-containing polynucleotide.

7. The method of claim 2, further comprising contacting said immobilized methylated complement template polynucleotide with a conversion agent thereby converting said one or more methylated cytosine nucleobases to one or more uracil or uracil analog nucleobases and generating an immobilized uracil-containing polynucleotide.

8. The method of claim 6, further comprising contacting said immobilized methylated complement template polynucleotide with a second conversion agent thereby converting said one or more methylated cytosine nucleobases to one or more 5-carboxylcytosine (5caC) nucleobases.

9. The method of claim 6, comprising annealing a primer to the immobilized uracil-containing polynucleotide and extending the primer hybridized to the immobilized uracil-containing polynucleotide with a polymerase to generate an amplification product.

10. The method of claim 5, further comprising vi) removing the chemical denaturant and hybridizing the immobilized methylated complement template polynucleotide to a second immobilized primer at the first temperature, wherein said second immobilized primer is attached to the solid support; and extending said second immobilized primer with a polymerase to generate an immobilized complement of the immobilized methylated complement template polynucleotide hybridized to the immobilized methylated complement template polynucleotide.

11. (canceled)

12. The method of claim 10, further comprising applying air to the solid support prior to removing the chemical denaturant.

13. The method of claim 10, further comprising vii) contacting the immobilized complement of the methylated complement template polynucleotide with a DNA methyltransferase reagent to generate an immobilized methylated template polynucleotide hybridized to said immobilized methylated complement template polynucleotide, wherein said immobilized methylated template polynucleotide comprises one or more methylated cytosine nucleobases and one or more non-methylated cytosine nucleobases, and contacting the immobilized methylated complement template polynucleotide with a conversion agent to convert the one or more non-methylated cytosine nucleobases to uracil nucleobases, thereby generating an immobilized uracil-containing polynucleotide comprising one or more uracil nucleobases.

14. The method of claim 13, further comprising repeating steps (i) to (vii), thereby amplifying the template polynucleotide.

15. (canceled)

16. The method of claim 1, wherein extending the first primer occurs at the first temperature, wherein the first temperature is about 40° C. to about 45° C.

17. The method of claim 2, wherein extending the first primer occurs at the second temperature, wherein the second temperature is about 55° C. to about 65° C.

18.-23. (canceled)

24. The method of claim 1, further comprising prior to step i) contacting the solid support with a sample comprising a methylated template polynucleotide.

25. The method of claim 1, wherein the DNA methyltransferase reagent comprises DNMT1.

26. (canceled)

27. (canceled)

28. The method of claim 9, further comprising contacting the amplification product with a chemical denaturant thereby separating the first polynucleotide and the amplification product; annealing a second immobilized primer to the second polynucleotide; and repeating step (iii).

29. (canceled)

30. (canceled)

31. The method of claim 1, further comprising sequencing the immobilized methylated complement template polynucleotide.

32.-35. (canceled)

36. An immobilized polynucleotide comprising a first nucleic acid sequence comprising one or more methylated cytosine nucleobases hybridized to a second nucleic acid sequence comprising one or more uracil nucleobases, wherein the polynucleotide comprises one or more cytosine mismatches, and wherein the immobilized polynucleotide is attached to a solid support.
